SEQUENCE LISTING (561 sequences)

SEQ ID NO: 1 - protein, 133 aa, unidentified [sequence omitted]

SEQ ID NO: 2 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 3 - protein, 133 aa, synthetic construct; identical to SEQ ID NO: 2
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 4 - protein, 133 aa, synthetic construct
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQKILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 5 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 6 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 7 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 8 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 9 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 10 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 11 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 12 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 13 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 14 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 15 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 16 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 17 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 18 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 19 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 20 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 21 - protein, 133 aa, synthetic construct
  SITE 11: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTKLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 22 - protein, 133 aa, synthetic construct
  SITE 13: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLKLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 23 - protein, 133 aa, synthetic construct
  SITE 15: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLKHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 24 - protein, 133 aa, synthetic construct
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLKDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 25 - protein, 133 aa, synthetic construct
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLKXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 26 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 27 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 28 - protein, 133 aa, synthetic construct; identical to SEQ ID NO: 2
  SITE 1: note = Attached to another sequence via 4x polyethylene glycol
  SITE 23: note = Norleucine
  SITE 32: note = Side chain attached to another sequence via a 1,5-pentanedioic acid-4x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 29 - protein, 133 aa, synthetic construct; identical to SEQ ID NO: 2
  SITE 1: note = Attached to another sequence via 16x polyethylene glycol
  SITE 23: note = Norleucine
  SITE 32: note = Side chain attached to another sequence via a 1,5-pentanedioic acid-16x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 30 - protein, 133 aa, synthetic construct; identical to SEQ ID NO: 2
  SITE 1: note = Attached to another sequence via 24x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 31 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..38: note = Residues linked via a 1,5-pentanedioic acid-9x polyethylene glycol linker
PLGLAGAPTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 32 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via 1,5-pentanedioic acid
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 33 - protein, 133 aa, synthetic construct
  SITE 1: note = Attached to another sequence via 9x polyethylene glycol
  SITE 23: note = Norleucine
  SITE 29: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 34 - protein, 133 aa, synthetic construct; identical to SEQ ID NO: 33
  SITE 1: note = Attached to another sequence via 24x polyethylene glycol
  SITE 23: note = Norleucine
  SITE 29: note = Side chain attached to another sequence via 24x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 35 - protein, 133 aa, synthetic construct; identical to SEQ ID NO: 33
  SITE 1: note = Attached to another sequence via 4x polyethylene glycol
  SITE 23: note = Norleucine
  SITE 29: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 36 - protein, 133 aa, synthetic construct; identical to SEQ ID NO: 33
  SITE 1: note = Attached to another sequence via 16x polyethylene glycol
  SITE 23: note = Norleucine
  SITE 29: note = Side chain attached to another sequence via 16x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 37 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..35: note = Residues linked via 9x polyethylene glycol
PLGLAGAPTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 38 - protein, 133 aa, synthetic construct; identical to SEQ ID NO: 33
  SITE 1: note = Attached to another sequence via 9x polyethylene glycol
  SITE 23: note = Norleucine
  SITE 29: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 39 - protein, 133 aa, synthetic construct
  SITE 1: note = Attached to another sequence via 4x polyethylene glycol
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 40 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..25: note = Residues linked via 4x polyethylene glycol
PLGLAGAPTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 41 - protein, 133 aa, synthetic construct
  SITE 1: note = N-terminus attached to another sequence via 4x polyethylene glycol
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 42 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..28: note = Residues linked via 4x polyethylene glycol
PLGLAGAPTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 43 - protein, 133 aa, synthetic construct
  SITE 1: note = Attached to another sequence via 9x polyethylene glycol
  SITE 23: note = Norleucine
  SITE 26: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 44 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..32: note = Residues linked via 9x polyethylene glycol
PLGLAGAPTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 45 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 46 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 47 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 48 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 49 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 50 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 51 - protein, 133 aa, synthetic construct
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 52 - protein, 133 aa, synthetic construct; identical to SEQ ID NO: 51
  SITE 9: note = Side chain attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 53 - protein, 133 aa, synthetic construct
  SITE 16: note = 2,4-diamino butyric acid
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEXLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 54 - protein, 133 aa, synthetic construct; identical to SEQ ID NO: 53
  SITE 16: note = N-(acetyl-glutyl-valinyl-citrullyl-(N-(4-aminobenzy l alcohol))-formyl)-L-lysine
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 55 - protein, 133 aa, synthetic construct; identical to SEQ ID NO: 2
  SITE 15: note = N-(acetyl-glutyl-valinyl-citrullyl-(N-(4-aminobenzy l alcohol)))ester-L-glutamate
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 56 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 57 - protein, 133 aa, synthetic construct; identical to SEQ ID NO: 51
  SITE 1: note = Attached to another sequence via 9x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 58 - protein, 140 aa, synthetic construct
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
  SITE 1..30: note = Residues linked via the side chain
GPLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 59 - protein, 139 aa, synthetic construct
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 4x polyethylene glycol
PLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 60 - protein, 139 aa, synthetic construct; identical to SEQ ID NO: 59
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 9x polyethylene glycol

SEQ ID NO: 61 - protein, 133 aa, synthetic construct; identical to SEQ ID NO: 4
  SITE 15: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 62 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 63 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 64 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 65 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 66 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 67 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 68 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 69 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 70 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 71 - protein, 143 aa, synthetic construct
  SITE 49: note = Norleucine
  SITE 51: note = Homoserine
  SITE 52: note = O-(9x polyethylene glycol-azido)-tyrosine
  SITE 55: note = O-(8x polyethylene glycol-amino)-tyrosine
  SITE 56: note = Norleucine
  SITE 81: note = Homoserine
  SITE 114: note = Homoserine
  SITE 1..33: note = Residues linked via the side chain
RGRRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 72 - protein, 144 aa, synthetic construct
  SITE 50: note = Norleucine
  SITE 52: note = Homoserine
  SITE 53: note = O-(9x polyethylene glycol-azido)-tyrosine
  SITE 56: note = O-(8x polyethylene glycol-amino)-tyrosine
  SITE 57: note = Norleucine
  SITE 82: note = Homoserine
  SITE 115: note = Homoserine
  SITE 1..34: note = Residues linked via the side chain
SGRVAPQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 73 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 74 - protein, 136 aa, synthetic construct
  SITE 26: note = (alpha-N-seryl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine
LVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 75 - protein, 136 aa, synthetic construct; identical to SEQ ID NO: 74
  SITE 26: note = (alpha-N-arginyl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 76 - protein, 136 aa, synthetic construct; identical to SEQ ID NO: 74
  SITE 26: note = Side chain attached to another sequence
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 77 - protein, 140 aa, synthetic construct
  SITE 30: note = (alpha-N-arginyl-glycyl-arginyl)-amido-L-glutamate
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
RVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 78 - protein, 133 aa, synthetic construct; identical to SEQ ID NO: 51
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 79 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 80 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 81 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 82 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 83 - protein, 133 aa, synthetic construct; identical to SEQ ID NO: 4
  SITE 1: note = N-terminus attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 84-100 - skipped (17 entries)

SEQ ID NO: 101 - protein, 15 aa, synthetic construct
SGGPGPAGMKGLPGS

SEQ ID NO: 102 - protein, 16 aa, synthetic construct
EAGRSANHEPLGLVAT

SEQ ID NO: 103 - protein, 10 aa, synthetic construct
PQASTGRSGG

SEQ ID NO: 104 - protein, 10 aa, synthetic construct
PQGSTGRAAG

SEQ ID NO: 105 - protein, 10 aa, synthetic construct
PPASSGRAGG

SEQ ID NO: 106 - protein, 16 aa, synthetic construct
RSGVPLSLYSGSGGGK

SEQ ID NO: 107 - protein, 14 aa, synthetic construct
RSGMPYDLYHPSGK

SEQ ID NO: 108 - protein, 14 aa, synthetic construct
RGPDSGGFMLTSGK

SEQ ID NO: 109 - protein, 15 aa, synthetic construct
RGSGHEQLTVSGGSK

SEQ ID NO: 110 - protein, 14 aa, synthetic construct
RSGRAAAVKSPSGK

SEQ ID NO: 111 - protein, 20 aa, synthetic construct
RGSGISSGLLSGRSDNHSGK

SEQ ID NO: 112 - protein, 14 aa, synthetic construct
RGDLLAVVAASGGK

SEQ ID NO: 113 - protein, 15 aa, synthetic construct
RGGISSGLLSGRSGK

SEQ ID NO: 114 - protein, 15 aa, synthetic construct
SGGGKKLADEPEGGS

SEQ ID NO: 115 - protein, 13 aa, synthetic construct
GGGKFLADEPEGG

SEQ ID NO: 116 - protein, 8 aa, synthetic construct
ARLQSAAP

SEQ ID NO: 117 - protein, 14 aa, synthetic construct
ARLQSAAPAGLKGA

SEQ ID NO: 118 - protein, 17 aa, synthetic construct
GSGGPGPAGMHGLPGGS

SEQ ID NO: 119 - protein, 16 aa, synthetic construct
GGGSHTGRSAYFGGGS

SEQ ID NO: 120 - protein, 15 aa, synthetic construct
SGGPGPAGLKGAPGS

SEQ ID NO: 121 - protein, 8 aa, synthetic construct
VPLSLYSG

SEQ ID NO: 122 - protein, 11 aa, synthetic construct
SGLLSGRSDNH

SEQ ID NO: 123 - protein, 11 aa, synthetic construct
SGRSDNIGGGS

SEQ ID NO: 124 - protein, 15 aa, synthetic construct
LQESLRSKESGRSDI

SEQ ID NO: 125 - protein, 13 aa, synthetic construct
ISSGLLSGRSDNH

SEQ ID NO: 126 - protein, 8 aa, synthetic construct
LSGRSDDH

SEQ ID NO: 127 - protein, 13 aa, synthetic construct
ISSGLLSGRSDQH

SEQ ID NO: 128 - protein, 13 aa, synthetic construct
ISSGLLSGRSDNI

SEQ ID NO: 129 - protein, 6 aa, synthetic construct
PLGLAG

SEQ ID NO: 130 - protein, 8 aa, synthetic construct
GPAGMKGL

SEQ ID NO: 131 - protein, 8 aa, synthetic construct
LSGRSDQH

SEQ ID NO: 132 - protein, 8 aa, synthetic construct
LSGRSDNI

SEQ ID NO: 133 - protein, 13 aa, synthetic construct
ISSGLLSGRSDNH

SEQ ID NO: 134 - protein, 7 aa, synthetic construct
GPLGVRG

SEQ ID NO: 135 - protein, 7 aa, synthetic construct
GPLGLAR

SEQ ID NO: 136 - protein, 8 aa, synthetic construct
GPAALVGA

SEQ ID NO: 137 - protein, 8 aa, synthetic construct
GPAALIGG

SEQ ID NO: 138 - protein, 8 aa, synthetic construct
GPLNLVGR

SEQ ID NO: 139 - protein, 8 aa, synthetic construct
GPAGLVAD

SEQ ID NO: 140 - protein, 8 aa, synthetic construct
GPANLVAP

SEQ ID NO: 141 - protein, 8 aa, synthetic construct
VPLSLYSG

SEQ ID NO: 142 - protein, 10 aa, synthetic construct
SGESPAYYTA

SEQ ID NO: 143 - protein, 7 aa, synthetic construct
GGPRGLG

SEQ ID NO: 144 - protein, 6 aa, synthetic construct
HSSKLQ

SEQ ID NO: 145 - skipped

SEQ ID NO: 146 - protein, 8 aa, synthetic construct
KRALGLPG

SEQ ID NO: 147 - protein, 5 aa, synthetic construct
LEATA

SEQ ID NO: 148 - protein, 10 aa, synthetic construct
GGAANLVRGG

SEQ ID NO: 149 - protein, 10 aa, synthetic construct
SGRIGFLRTA

SEQ ID NO: 150 - protein, 5 aa, synthetic construct
PLGLA

SEQ ID NO: 151 - protein, 8 aa, synthetic construct
ESPAYYTA

SEQ ID NO: 152 - protein, 6 aa, synthetic construct
RLQLKL

SEQ ID NO: 153 - protein, 7 aa, synthetic construct
RLQLKAC

SEQ ID NO: 154 - protein, 5 aa, synthetic construct
SGRSA

SEQ ID NO: 155 - protein, 4 aa, synthetic construct
DAFK

SEQ ID NO: 156 - protein, 5 aa, synthetic construct
GGGRR

SEQ ID NO: 157 - protein, 4 aa, synthetic construct
GFLG

SEQ ID NO: 158 - protein, 9 aa, synthetic construct
HSSKLQEDA

SEQ ID NO: 159 - protein, 10 aa, synthetic construct
LVLASSSFGY

SEQ ID NO: 160 - protein, 10 aa, synthetic construct
GVSQNYPIVG

SEQ ID NO: 161 - protein, 10 aa, synthetic construct
GVVQASCRLA

SEQ ID NO: 162 - protein, 6 aa, synthetic construct
DPRSFL

SEQ ID NO: 163 - protein, 6 aa, synthetic construct
PPRSFL

SEQ ID NO: 164 - protein, 4 aa, synthetic construct
DEVD

SEQ ID NO: 165 - protein, 5 aa, synthetic construct
DEVDP

SEQ ID NO: 166 - protein, 8 aa, synthetic construct
KGSGDVEG

SEQ ID NO: 167 - protein, 6 aa, synthetic construct
GWEHDG

SEQ ID NO: 168 - protein, 7 aa, synthetic construct
EDDDDKA

SEQ ID NO: 169 - protein, 9 aa, synthetic construct
KQEQNPGST

SEQ ID NO: 170 - protein, 6 aa, synthetic construct
GKAFRR

SEQ ID NO: 171 - protein, 4 aa, synthetic construct
DAFK

SEQ ID NO: 172 - protein, 4 aa, synthetic construct
DVLK

SEQ ID NO: 173 - protein, 4 aa, synthetic construct
DAFK

SEQ ID NO: 174 - protein, 7 aa, synthetic construct
ALLLALL

SEQ ID NO: 175 - protein, 10 aa, synthetic construct
SGAKPRALTA

SEQ ID NO: 176 - protein, 10 aa, synthetic construct
SGLRLAAITA

SEQ ID NO: 177 - protein, 10 aa, synthetic construct
SGESLAYYTA

SEQ ID NO: 178 - protein, 10 aa, synthetic construct
SGESPAYYTA

SEQ ID NO: 179 - protein, 10 aa, synthetic construct
SGESLRYYTA

SEQ ID NO: 180 - protein, 10 aa, synthetic construct
SGRSLSRLTA

SEQ ID NO: 181 - protein, 10 aa, synthetic construct
SGRSLRRLTA

SEQ ID NO: 182 - protein, 10 aa, synthetic construct
SGAVSWLLTA

SEQ ID NO: 183 - protein, 10 aa, synthetic construct
SGAPSWLLTA

SEQ ID NO: 184 - protein, 10 aa, synthetic construct
SGAVRWLLTA

SEQ ID NO: 185 - protein, 10 aa, synthetic construct
SGANISDLTA

SEQ ID NO: 186 - protein, 10 aa, synthetic construct
SGNRYSSLTA

SEQ ID NO: 187 - protein, 10 aa, synthetic construct
SGHMHKALTA

SEQ ID NO: 188 - protein, 10 aa, synthetic construct
SGHMHKALTA

SEQ ID NO: 189-200 - skipped (12 entries)

SEQ ID NO: 201 - protein, 11 aa, synthetic construct
AGDKSPLGLAG

SEQ ID NO: 202 - protein, 11 aa, synthetic construct
AGDRSPLGLAG

SEQ ID NO: 203 - protein, 12 aa, synthetic construct
AGDRSAPLGLAG

SEQ ID NO: 204 - protein, 11 aa, synthetic construct
AWGRSPLGLAG

SEQ ID NO: 205 - protein, 12 aa, synthetic construct
AWGRSAPLGLAG

SEQ ID NO: 206 - protein, 11 aa, synthetic construct
AWGKSPLGLAG

SEQ ID NO: 207 - protein, 11 aa, synthetic construct
GAFKSPLGLAG

SEQ ID NO: 208 - protein, 11 aa, synthetic construct
SGRSAPLALAG

SEQ ID NO: 209 - protein, 10 aa, synthetic construct
SGRSPLGLAG

SEQ ID NO: 210-213 - skipped (4 entries)

SEQ ID NO: 214 - protein, 12 aa, synthetic construct
RSGRSAPLGLAG

SEQ ID NO: 215 - protein, 12 aa, synthetic construct
RSGKSAPLGLAG

SEQ ID NO: 216 - protein, 12 aa, synthetic construct
FTARSAPLGLAG

SEQ ID NO: 217 - skipped

SEQ ID NO: 218 - protein, 11 aa, synthetic construct
FTAKSPLGLAG

SEQ ID NO: 219 - protein, 12 aa, synthetic construct
RYGRSAPLGLAG

SEQ ID NO: 220 - protein, 11 aa, synthetic construct
RYGRSPLGLAG

SEQ ID NO: 221 - skipped

SEQ ID NO: 222 - protein, 12 aa, synthetic construct
KYGRSAPLGLAG

SEQ ID NO: 223 - protein, 12 aa, synthetic construct
KWGRSAPLGLAG

SEQ ID NO: 224 - skipped

SEQ ID NO: 225 - protein, 11 aa, synthetic construct
KWGRSPLGLAG

SEQ ID NO: 226-235 - skipped (10 entries)

SEQ ID NO: 236 - protein, 13 aa, synthetic construct
SSRGRRGPLGLAG

SEQ ID NO: 237 - protein, 10 aa, synthetic construct
SSRGPLGLAG

SEQ ID NO: 238 - protein, 10 aa, synthetic construct
SSRGPRGLAG

SEQ ID NO: 239-245 - skipped (7 entries)

SEQ ID NO: 246 - protein, 11 aa, synthetic construct
SGRVLPLNLSG

SEQ ID NO: 247-249 - skipped (3 entries)

SEQ ID NO: 250 - protein, 11 aa, synthetic construct
SGRVLPLGLAG

SEQ ID NO: 251 - protein, 11 aa, synthetic construct
SGRVLPMSLRM

SEQ ID NO: 252 - skipped

SEQ ID NO: 253 - protein, 11 aa, synthetic construct
SGRVLPLGLRA

SEQ ID NO: 254 - skipped

SEQ ID NO: 255 - protein, 11 aa, synthetic construct
SGRVLPLGFMG

SEQ ID NO: 256 - protein, 12 aa, synthetic construct
GEEGEEPLGLAG

SEQ ID NO: 257 - protein, 7 aa, synthetic construct
GPLGLAG

SEQ ID NO: 258 - protein, 16 aa, synthetic construct
EAGRSANHTPAGLTGP

SEQ ID NO: 259 - protein, 17 aa, synthetic construct
GEAGRSANHTPAGLTGP

SEQ ID NO: 260-300 - skipped (41 entries)

SEQ ID NO: 301 - protein, 17 aa, synthetic construct
SSRGPASNRRLPLGLAG

SEQ ID NO: 302 - protein, 17 aa, synthetic construct
SSRAVFRKNLGPLGLAG

SEQ ID NO: 303 - protein, 15 aa, synthetic construct
SGRVLRKAGPQPLVD

SEQ ID NO: 304 - protein, 16 aa, synthetic construct
SGRVLTLRKAPWGLLE

SEQ ID NO: 305 - protein, 12 aa, synthetic construct
SGRVLGPAGLVG

SEQ ID NO: 306 - protein, 12 aa, synthetic construct
SGRVLGPANLVG

SEQ ID NO: 307 - protein, 11 aa, synthetic construct
SGRVLPAGLVG

SEQ ID NO: 308 - protein, 12 aa, synthetic construct
SGRVLGPLNLSG

SEQ ID NO: 309 - protein, 12 aa, synthetic construct
RQRRSAPLGLAG

SEQ ID NO: 310 - protein, 11 aa, synthetic construct
RQRRSPLGLAG

SEQ ID NO: 311 - protein, 17 aa, synthetic construct
SGRVLTLRKAGPAGLVG

SEQ ID NO: 312 - protein, 17 aa, synthetic construct
SGRVLTLRKAGPANLVG

SEQ ID NO: 313 - protein, 15 aa, synthetic construct
SGRVLRKAGPAGLVG

SEQ ID NO: 314 - protein, 15 aa, synthetic construct
SGRVLRKAGPANLVG

SEQ ID NO: 315 - protein, 11 aa, synthetic construct
SGRVLPANLVG

SEQ ID NO: 316 - protein, 9 aa, synthetic construct
SGRVAGLVG

SEQ ID NO: 317 - protein, 9 aa, synthetic construct
SGRVANLVG

SEQ ID NO: 318 - protein, 13 aa, synthetic construct
PASNRRLPLGLAG

SEQ ID NO: 319 - protein, 13 aa, synthetic construct
SSRVFRKPANLAG

SEQ ID NO: 320 - protein, 16 aa, synthetic construct
SGRVLTLRKAALPLAM

SEQ ID NO: 321 - protein, 13 aa, synthetic construct
SSRGRRGPYMLQG

SEQ ID NO: 322 - protein, 10 aa, synthetic construct
SSRGPYMLQG

SEQ ID NO: 323 - protein, 11 aa, synthetic construct
SGRVLPLGMRA

SEQ ID NO: 324 - protein, 11 aa, synthetic construct
SGRVLPYAMTA

SEQ ID NO: 325 - protein, 12 aa, synthetic construct
  SITE 6: note = Norleucine
RQRRSXPLGLAG

SEQ ID NO: 326 - protein, 12 aa, synthetic construct
KWGKSAPLGLAG

SEQ ID NO: 327 - protein, 12 aa, synthetic construct
RYGKSAPLGLAG

SEQ ID NO: 328 - protein, 13 aa, synthetic construct
RQRRSAAPLGLAG

SEQ ID NO: 329 - protein, 9 aa, synthetic construct
RQRRSVVGG

SEQ ID NO: 330 - protein, 8 aa, synthetic construct
SPLGLAGS

SEQ ID NO: 331 - protein, 10 aa, synthetic construct
RGRKVANLVG

SEQ ID NO: 332 - protein, 10 aa, synthetic construct
RQRKVANLVG

SEQ ID NO: 333 - protein, 10 aa, synthetic construct
RGRRVANLVG

SEQ ID NO: 334 - protein, 11 aa, synthetic construct
RGRKSPANLVG

SEQ ID NO: 335 - protein, 10 aa, synthetic construct
RGRKPYMLQG

SEQ ID NO: 336 - protein, 10 aa, synthetic construct
  SITE 7: note = Norleucine
RGRKPYXLQG

SEQ ID NO: 337 - protein, 11 aa, synthetic construct
RGRKSPYMLQG

SEQ ID NO: 338 - protein, 11 aa, synthetic construct
  SITE 8: note = Norleucine
RGRKSPYXLQG

SEQ ID NO: 339 - protein, 10 aa, synthetic construct
RGRKPQPLVD

SEQ ID NO: 340 - protein, 11 aa, synthetic construct
RGRKSPQPLVD

SEQ ID NO: 341 - protein, 10 aa, synthetic construct
RGRKSQPLVD

SEQ ID NO: 342 - protein, 11 aa, synthetic construct
SGRVAPYMLQG

SEQ ID NO: 343 - protein, 11 aa, synthetic construct
  SITE 8: note = Norleucine
SGRVAPYXLQG

SEQ ID NO: 344 - protein, 9 aa, synthetic construct
SGRVYMLQG

SEQ ID NO: 345 - protein, 9 aa, synthetic construct
  SITE 6: note = Norleucine
SGRVYXLQG

SEQ ID NO: 346 - protein, 11 aa, synthetic construct
SGRVAPQPLVD

SEQ ID NO: 347 - protein, 9 aa, synthetic construct
SGRVQPLVD

SEQ ID NO: 348 - protein, 6 aa, synthetic construct
RGRRGP

SEQ ID NO: 349 - protein, 10 aa, synthetic construct
RGRRPLGLAG

SEQ ID NO: 350 - protein, 15 aa, synthetic construct
RGRRVANPLGLAGSG

SEQ ID NO: 351 - protein, 13 aa, synthetic construct
RGRRPLGLAGGSG

SEQ ID NO: 352 - protein, 10 aa, synthetic construct
RGRRHSSKLQ

SEQ ID NO: 353 - protein, 12 aa, synthetic construct
SGRVANPLGGSG

SEQ ID NO: 354 - protein, 11 aa, synthetic construct
SGRVANYFGKL

SEQ ID NO: 355 - protein, 12 aa, synthetic construct
RGRRVANYFGKL

SEQ ID NO: 356 - protein, 11 aa, synthetic construct
SGRPLGYFGKL

SEQ ID NO: 357 - protein, 12 aa, synthetic construct
RGRRPLGYFGKL

SEQ ID NO: 358 - protein, 15 aa, synthetic construct
RGRRVANPLGYFGKL

SEQ ID NO: 359 - protein, 21 aa, synthetic construct
RGRRSGRAANLVRPLGYFGKL

SEQ ID NO: 360 - protein, 18 aa, synthetic construct
RGRRAANLVRPLGYFGKL

SEQ ID NO: 361 - protein, 11 aa, synthetic construct
HSSKLQYFGKL

SEQ ID NO: 362 - protein, 18 aa, synthetic construct
RGRRHSSKLQPLGYFGKL

SEQ ID NO: 363 - protein, 17 aa, synthetic construct
SGRHSSKLQPLGYFGKL

SEQ ID NO: 364 - protein, 8 aa, synthetic construct
GSGSGSGS

SEQ ID NO: 365 - protein, 8 aa, synthetic construct
SSLYSSPG

SEQ ID NO: 366 - protein, 8 aa, synthetic construct
SSLQSSPG

SEQ ID NO: 367 - protein, 8 aa, synthetic construct
SQYQSSPG

SEQ ID NO: 368 - protein, 8 aa, synthetic construct
SQLYSSPG

SEQ ID NO: 369 - protein, 8 aa, synthetic construct
SSQYSSPG

SEQ ID NO: 370 - protein, 8 aa, synthetic construct
ISQYSSAT

SEQ ID NO: 371 - protein, 7 aa, synthetic construct
KLYSSKQ

SEQ ID NO: 372 - protein, 7 aa, synthetic construct
KLFSSKQ

SEQ ID NO: 373 - protein, 7 aa, synthetic construct
RRLHYSL

SEQ ID NO: 374 - protein, 7 aa, synthetic construct
RRLNYSL

SEQ ID NO: 375 - protein, 7 aa, synthetic construct
RSSYRSL

SEQ ID NO: 376 - protein, 7 aa, synthetic construct
RSSYYSL

SEQ ID NO: 377 - protein, 7 aa, synthetic construct
KSKQHSL

SEQ ID NO: 378 - protein, 7 aa, synthetic construct
HSSKLQL

SEQ ID NO: 379 - protein, 8 aa, synthetic construct
GSSYYSGA

SEQ ID NO: 380 - protein, 8 aa, synthetic construct
GSSVYSGR

SEQ ID NO: 381 - protein, 8 aa, synthetic construct
  SITE 3: note = Norleucine
SSXYSSAG

SEQ ID NO: 382-400 - skipped (19 entries)

SEQ ID NO: 401 - protein, 4 aa, synthetic construct
SSRV

SEQ ID NO: 402 - protein, 4 aa, synthetic construct
SSRG

SEQ ID NO: 403 - protein, 4 aa, synthetic construct
SGRV

SEQ ID NO: 404 - protein, 4 aa, synthetic construct
RKAG

SEQ ID NO: 405 - protein, 4 aa, synthetic construct
RQRR

SEQ ID NO: 406 - protein, 4 aa, synthetic construct
RGRR

SEQ ID NO: 407 - protein, 4 aa, synthetic construct
RGRK

SEQ ID NO: 408 - protein, 4 aa, synthetic construct
RQRK

SEQ ID NO: 409 - protein, 4 aa, synthetic construct
GKSA

SEQ ID NO: 410 - protein, 4 aa, synthetic construct
MLQG

SEQ ID NO: 411 - skipped

SEQ ID NO: 412 - protein, 4 aa, synthetic construct
PASN

SEQ ID NO: 413 - protein, 4 aa, synthetic construct
PLVD

SEQ ID NO: 414 - protein, 4 aa, synthetic construct
SSRA

SEQ ID NO: 415 - protein, 5 aa, synthetic construct
RQRRS

SEQ ID NO: 416 - protein, 5 aa, synthetic construct
RGRKS

SEQ ID NO: 417 - protein, 5 aa, synthetic construct
RGRKP

SEQ ID NO: 418 - protein, 5 aa, synthetic construct
SGRVA

SEQ ID NO: 419 - protein, 5 aa, synthetic construct
SGRVY

SEQ ID NO: 420 - protein, 5 aa, synthetic construct
SGGPG

SEQ ID NO: 421 - protein, 5 aa, synthetic construct
AGLVG

SEQ ID NO: 422 - protein, 5 aa, synthetic construct
ANLVG

SEQ ID NO: 423 - protein, 5 aa, synthetic construct
SGRVL

SEQ ID NO: 424 - protein, 5 aa, synthetic construct
RQRRV

SEQ ID NO: 425 - protein, 6 aa, synthetic construct
PQPLVD

SEQ ID NO: 426 - protein, 6 aa, synthetic construct
PWGLLE

SEQ ID NO: 427 - protein, 6 aa, synthetic construct
PAGLVG

SEQ ID NO: 428 - protein, 6 aa, synthetic construct
PANLVG

SEQ ID NO: 429 - protein, 6 aa, synthetic construct
PLNLSG

SEQ ID NO: 430 - protein, 5 aa, synthetic construct
AGLVG

SEQ ID NO: 431 - protein, 5 aa, synthetic construct
ANLVG

SEQ ID NO: 432 - protein, 6 aa, synthetic construct
PANLAG

SEQ ID NO: 433 - protein, 6 aa, synthetic construct
GEEGEE

SEQ ID NO: 434 - protein, 6 aa, synthetic construct
MKGLPG

SEQ ID NO: 435 - protein, 6 aa, synthetic construct
RGRKSP

SEQ ID NO: 436 - protein, 6 aa, synthetic construct
RGRKPY

SEQ ID NO: 437 - protein, 6 aa, synthetic construct
RGRKPQ

SEQ ID NO: 438 - protein, 6 aa, synthetic construct
PAGLTP

SEQ ID NO: 439 - protein, 6 aa, synthetic construct
PAGLTG

SEQ ID NO: 440 - protein, 9 aa, synthetic construct
EAGRSANHT

SEQ ID NO: 441 - protein, 7 aa, synthetic construct
MKGLPGS

SEQ ID NO: 442 - protein, 7 aa, synthetic construct
KPLGLAG

SEQ ID NO: 443 - protein, 8 aa, synthetic construct
KKPLGLAG

SEQ ID NO: 444 - protein, 16 aa, synthetic construct
GEAGRSANHTPAGLTP

SEQ ID NO: 445-501 - skipped (57 entries)

SEQ ID NO: 502 - protein, 6 aa, synthetic construct
  SITE 1: note = Acetylated residue
  SITE 6: note = C-terminus attached to another sequence
PLGLAG

SEQ ID NO: 503 - protein, 6 aa, synthetic construct
  SITE 6: note = C-terminus attached to another sequence
PLGLAG

SEQ ID NO: 504 - protein, 6 aa, synthetic construct
  SITE 1: note = Acetylated residue
  SITE 6: note = C-terminus attached to another sequence via 9x polyethylene glycol
PLGLAG

SEQ ID NO: 505 - protein, 6 aa, synthetic construct
  SITE 6: note = C-terminus attached to another sequence via 9x polyethylene glycol
PLGLAG

SEQ ID NO: 506 - protein, 15 aa, synthetic construct
  SITE 1: note = Acetylated residue
  SITE 15: note = C-terminus attached to another sequence
SGGPGPAGMKGLPGS

SEQ ID NO: 507 - protein, 7 aa, synthetic construct
  SITE 7: note = C-terminus attached to another sequence
MKGLPGS

SEQ ID NO: 508 - protein, 15 aa, synthetic construct
  SITE 15: note = C-terminus attached to another sequence
SGGPGPAGMKGLPGS

SEQ ID NO: 509 - protein, 15 aa, synthetic construct
  SITE 1: note = Acetylated residue
  SITE 15: note = C-terminus attached to another sequence via 9x polyethylene glycol
SGGPGPAGMKGLPGS

SEQ ID NO: 510 - protein, 7 aa, synthetic construct
  SITE 7: note = C-terminus attached to another sequence via 9x polyethylene glycol
MKGLPGS

SEQ ID NO: 511 - protein, 15 aa, synthetic construct
  SITE 15: note = C-terminus attached to another sequence via 9x polyethylene glycol
SGGPGPAGMKGLPGS

SEQ ID NO: 512 - protein, 6 aa, synthetic construct
  SITE 6: note = C-terminus attached to another sequence
PLGLAG

SEQ ID NO: 513 - protein, 6 aa, synthetic construct
  SITE 6: note = C-terminus attached to another sequence
PLGLAG

SEQ ID NO: 514 - protein, 12 aa, synthetic construct
  SITE 12: note = C-terminus attached to another sequence
GEEGEEPLGLAG

SEQ ID NO: 515 - protein, 7 aa, synthetic construct
  SITE 1: note = Lysine attached to 27x polyethylene glycol
  SITE 7: note = C-terminus attached to another sequence
KPLGLAG

SEQ ID NO: 516 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKPLGLAG

SEQ ID NO: 517 - protein, 6 aa, synthetic construct
  SITE 6: note = C-terminus attached to another sequence
PLGLAG

SEQ ID NO: 518 - protein, 6 aa, synthetic construct
  SITE 6: note = C-terminus attached to another sequence
PLGLAG

SEQ ID NO: 519 - protein, 6 aa, synthetic construct
  SITE 6: note = C-terminus attached to another sequence
PLGLAG

SEQ ID NO: 520 - protein, 6 aa, synthetic construct
  SITE 6: note = C-terminus attached to another sequence
PLGLAG

SEQ ID NO: 521 - protein, 6 aa, synthetic construct
  SITE 6: note = C-terminus attached to another sequence
PLGLAG

SEQ ID NO: 522 - protein, 16 aa, synthetic construct
  SITE 16: note = C-terminus attached to another sequence
EAGRSANHTPAGLTGP

SEQ ID NO: 523 - protein, 6 aa, synthetic construct
  SITE 1: note = Attached to another sequence via a 1,5-pentanedioic acid-9x polyethylene glycol linker
  SITE 6: note = Attached to another sequence via 9x polyethylene glycol
PLGLAG

SEQ ID NO: 524 - protein, 15 aa, synthetic construct
  SITE 1: note = Attached to another sequence via a 1,5-pentanedioic acid-4x polyethylene glycol linker
  SITE 15: note = Attached to another sequence via 4x polyethylene glycol
SGGPGPAGMKGLPGS

SEQ ID NO: 525 - protein, 15 aa, synthetic construct
  SITE 1: note = Attached to another sequence via a 1,5-pentanedioic acid-16x polyethylene glycol linker
  SITE 15: note = Attached to another sequence via 16x polyethylene glycol
SGGPGPAGMKGLPGS

SEQ ID NO: 526 - protein, 6 aa, synthetic construct
  SITE 1: note = Attached to another sequence via a 1,5-pentanedioic acid-9x polyethylene glycol linker
  SITE 6: note = Attached to another sequence via 24x polyethylene glycol
PLGLAG

SEQ ID NO: 527 - protein, 7 aa, synthetic construct
  SITE 1: note = Attached to another sequence via 1,5-pentanedioic acid
  SITE 7: note = Attached to another sequence via 9x polyethylene glycol
GPLGLAG

SEQ ID NO: 528 - protein, 6 aa, synthetic construct
  SITE 1: note = Attached to another sequence via 9x polyethylene glycol
  SITE 6: note = Attached to another sequence via 9x polyethylene glycol
PLGLAG

SEQ ID NO: 529 - protein, 15 aa, synthetic construct
  SITE 1: note = Attached to another sequence via 24x polyethylene glycol
  SITE 15: note = Attached to another sequence via 24x polyethylene glycol
SGGPGPAGMKGLPGS

SEQ ID NO: 530 - protein, 15 aa, synthetic construct
  SITE 1: note = Attached to another sequence via 4x polyethylene glycol
  SITE 15: note = Attached to another sequence via 4x polyethylene glycol
SGGPGPAGMKGLPGS

SEQ ID NO: 531 - protein, 15 aa, synthetic construct
  SITE 1: note = Attached to another sequence via 16x polyethylene glycol
  SITE 15: note = Attached to another sequence via 16x polyethylene glycol
SGGPGPAGMKGLPGS

SEQ ID NO: 532 - protein, 7 aa, synthetic construct
  SITE 1: note = N-terminus attached to another sequence
  SITE 7: note = Attached to another sequence via 9x polyethylene glycol
GPLGLAG

SEQ ID NO: 533 - protein, 7 aa, synthetic construct
  SITE 1: note = N-terminus attached to another sequence
  SITE 7: note = Attached to another sequence via 4x polyethylene glycol
GPLGLAG

SEQ ID NO: 534 - protein, 7 aa, synthetic construct
  SITE 1: note = N-terminus attached to another sequence
  SITE 7: note = Attached to another sequence via 4x polyethylene glycol
GPLGLAG

SEQ ID NO: 535 - protein, 7 aa, synthetic construct
  SITE 1: note = N-terminus attached to another sequence
  SITE 7: note = Attached to another sequence via 9x polyethylene glycol
GPLGLAG

SEQ ID NO: 536 - protein, 6 aa, synthetic construct
  SITE 1: note = Attached to another sequence via a 1,5-pentanedioic acid-9x polyethylene glycol linker
  SITE 6: note = C-terminus attached to another sequence
PLGLAG

SEQ ID NO: 537 - protein, 6 aa, synthetic construct
  SITE 1: note = Attached to another sequence via a 1,5-pentanedioic acid-4x polyethylene glycol linker
  SITE 6: note = C-terminus attached to another sequence
PLGLAG

SEQ ID NO: 538 - protein, 7 aa, synthetic construct
  SITE 1: note = Attached to another sequence via 1,5-pentanedioic acid
  SITE 7: note = C-terminus attached to another sequence
GPLGLAG

SEQ ID NO: 539 - protein, 17 aa, synthetic construct
  SITE 1: note = Attached to another sequence via 1,5-pentanedioic acid
  SITE 17: note = C-terminus attached to another sequence
GEAGRSANHTPAGLTGP

SEQ ID NO: 540 - protein, 16 aa, synthetic construct
  SITE 1: note = Attached to another sequence via a 1,5-pentanedioic acid-4x polyethylene glycol linker
  SITE 16: note = C-terminus attached to another sequence
EAGRSANHTPAGLTGP

SEQ ID NO: 541 - protein, 16 aa, synthetic construct
  SITE 1: note = Attached to another sequence via a 1,5-pentanedioic acid-9x polyethylene glycol linker
  SITE 16: note = C-terminus attached to another sequence
EAGRSANHTPAGLTGP

SEQ ID NO: 542 - protein, 6 aa, synthetic construct
  SITE 1: note = N-terminus attached to another sequence
  SITE 6: note = Attached to another sequence via 4x polyethylene glycol
PLGLAG

SEQ ID NO: 543 - protein, 6 aa, synthetic construct
  SITE 1: note = Attached to another sequence via 4x polyethylene glycol
  SITE 6: note = C-terminus attached to another sequence
PLGLAG

SEQ ID NO: 544 - protein, 7 aa, synthetic construct
  SITE 1: note = N-terminus attached to another sequence
  SITE 7: note = Attached to another sequence via 4x polyethylene glycol
GPLGLAG

SEQ ID NO: 545 - protein, 7 aa, synthetic construct
  SITE 1: note = N-terminus attached to another sequence
  SITE 7: note = Attached to another sequence via 9x polyethylene glycol
GPLGLAG

SEQ ID NO: 546 - protein, 7 aa, synthetic construct
  SITE 1: note = N-terminus attached to another sequence
  SITE 7: note = C-terminus attached to another sequence
GPLGLAG

SEQ ID NO: 547 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKGSGSGS

SEQ ID NO: 548 - protein, 11 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 11: note = C-terminus attached to another sequence
KKSGRVANLVG

SEQ ID NO: 549 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVANLVG

SEQ ID NO: 550 - protein, 12 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 12: note = C-terminus attached to another sequence
KKRGRRVANLVG

SEQ ID NO: 551 - protein, 12 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 12: note = C-terminus attached to another sequence
KKRGRRVANLVG

SEQ ID NO: 552 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVQPLVD

SEQ ID NO: 553 - protein, 10 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 10: note = C-terminus attached to another sequence
KKGSGSGSGS

SEQ ID NO: 554 - protein, 7 aa, synthetic construct
  SITE 1: note = N-terminus attached to another sequence
  SITE 7: note = Attached to another sequence via 4x polyethylene glycol
GSGSGSG

SEQ ID NO: 555 - protein, 7 aa, synthetic construct
  SITE 1: note = N-terminus attached to another sequence
RGRRVAN

SEQ ID NO: 556 - protein, 6 aa, synthetic construct
  SITE 1: note = N-terminus attached to another sequence via 4x polyethylene glycol
  SITE 6: note = C-terminus attached to another sequence
GSGSGS

SEQ ID NO: 557 - protein, 9 aa, synthetic construct
  SITE 1: note = N-terminus attached to another sequence
  SITE 9: note = C-terminus attached to another sequence
SGRVANLVG

SEQ ID NO: 558 - protein, 10 aa, synthetic construct
  SITE 1: note = N-terminus attached to another sequence
  SITE 10: note = C-terminus attached to another sequence
RGRRVANLVG

SEQ ID NO: 559 - protein, 11 aa, synthetic construct
  SITE 1: note = N-terminus attached to another sequence
  SITE 11: note = C-terminus attached to another sequence
SGRVAPQPLVD

SEQ ID NO: 560 - protein, 9 aa, synthetic construct
  SITE 1: note = N-terminus attached to another sequence
  SITE 9: note = C-terminus attached to another sequence
SGRVQPLVD

SEQ ID NO: 561 - protein, 6 aa, synthetic construct
  SITE 1: note = Attached to another sequence via a 1,5-pentanedioic acid-9x polyethylene glycol linker
  SITE 6: note = C-terminus attached to another sequence
GSGSGS

(19) United States
(12) Patent Application Publication
(10) Pub. No.: US 2024/0400614 A1
(43) Pub. Date: Dec. 5, 2024

(54) CLEAVABLE PEPTIDES AND METHODS OF USE THEREOF

(71) Applicant: Bright Peak Therapeutics AG, Basel (CH)

(72) Inventors: Bertolt KREFT, Kleinmachnow (DE); Vijaya Raghavan PATTABIRAMAN, Volketswil (CH); Arnaud GOEPFERT, Sierentz (FR); Grégory UPERT, Kembs (FR); Tiziano ONGARO, Basel (CH); Matilde ARÉVALO-RUIZ, Basel (CH)

(21) Appl. No.: 18/410,854

(22) Filed: Jan. 11, 2024

Related U.S. Application Data
(60) Provisional application No. 63/438,456, filed on Jan. 11, 2023.
(60) Provisional application No. 63/438,396, filed on Jan. 11, 2023.
(60) Provisional application No. 63/438,468, filed on Jan. 11, 2023.

(51) Int. Cl. C07K 7/06

(52) U.S. Cl. CPC C07K 7/06

(57) ABSTRACT

The present disclosure relates to cleavable peptides, including multi-protease cleavable peptides, and methods of use thereof. The cleavable peptides are useful in a variety of applications, including as conditional linkers between two groups and as conditional activators of proteins. The present disclosure also relates to a composition of conditionally regulated proteins with cleavable peptides for treatment of diseases including but not limited to cancer. The conditional activation of proteins is based on cleavable polypeptides that regulate the protein activity.

### FIELD OF THE INVENTION

The invention also relates to cleavable peptides, including peptides cleavable by multiple proteases. Such cleavable peptides can be used in conditionally regulated proteins or can be used to conditionally attach different groups by using the cleavable peptide as a linker. The invention also relates to the composition of conditionally regulated proteins with cleavable peptides for treatment of diseases including but not limited to cancer. The conditional activation of proteins is based on cleavable polypeptides that regulate the protein activity. The invention also provides methods of attachment of different linkers to one or more protein molecules.

### CROSS REFERENCE

This application claims the benefit of U.S. Provisional Application No. 63/438,396 filed Jan. 11, 2023, U.S. Provisional Application No. 63/438,456, filed Jan. 11, 2023, and U.S. Provisional Application No. 63/438,468, filed Jan. 11, 2023, each of which applications are incorporated herein by reference in its entirety.

### SEQUENCE LISTING

The instant application contains a Sequence Listing which has been submitted electronically in XML format and is hereby incorporated by reference in its entirety. Said XML copy, created on Jan. 31, 2024, is named 94917-0111_734201US_SL.xml and is 696,492 bytes in size.

### BACKGROUND

Therapeutic proteins are important molecules in the treatment of diseases such as cancer, microbial infections, and exocrine diseases. Antibody therapies and therapeutic proteins like cytokines have been used to activate the immune system towards certain cancers, both as monotherapies and in combination. However, the use of protein therapies as treatment for disease has been limited by off target effects. Such off target effects include vascular leak syndrome in IL-2 therapies and the development of autoimmune diseases like type-1-diabetes in anti-PD1 antibody therapies. There exists a need for improved therapeutic proteins where off target effects can be minimized and the therapeutic proteins can be conditionally activated. Selective activation of therapeutic proteins is likely to diversify and enhance their therapeutic potential.

### SUMMARY OF THE INVENTION

In one aspect, the invention relates to cleavable peptides. In some embodiments, the cleavable peptides are multi-protease cleavable peptides (i.e., peptides cleavable by two or more different proteases). In some embodiments, the cleavable peptide is a multi-protease cleavable peptide cleavable by two or more proteases associated with upregulation in or near a tumor or tumor microenvironment. In some embodiments, the cleavable peptides are used as linkers between two groups (e.g., between two proteins, between a protein and a polymer, between a protein and a drug molecule (e.g., an antibody-drug conjugate), between a nanoparticle and a drug, between a targeting moiety (e.g., an aptamer) and a drug, or any other two groups desired to be conditionally linked). In some embodiments, a cleavable peptide is incorporated as a linker into the sequence of a protein to allow cleavage (and, in some embodiments, activation) of the protein. In some embodiments, a cleavable peptide is incorporated into a fusion protein as a linker between the two fused genes (e.g., between a receptor binding protein in a dummy receptor, wherein cleavage of the cleavable peptide causes the dummy receptor to disassociate with the receptor binding protein and allow the receptor binding protein to interact with its cognate receptor, or between a receptor binding protein and a bulky protein group (e.g., an albumin polypeptide, an Fc polypeptide, etc), wherein cleavage of the cleavable peptide causes the bulky protein to dissociate with the receptor binding protein and allow the receptor binding protein to interact with its cognate receptor). In some embodiments, a cleavable peptide is attached to a protein in such a manner that the cleavable peptide itself, when intact, blocks or reduces interaction of the protein with a ligand of the protein (e.g., with a cognate receptor of the protein).

In one aspect, provided herein, is a cleavable peptide having a formula G², wherein G² has a structure of the formula: *—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—** wherein X¹ is S, R, P, K, F, G, or absent; X² is S, G, Q, A, W, Y, T, E or absent; X³ is R, S, G, A, E, or absent; X⁴ is G, A, V, R, N, K, P, or absent; X⁵ is P, V, L, S, F, R, A, Y, Q, G, E or absent; X⁶ is A, F, R, T, G, P, Nle, V, Y, Q, E, S, or absent; X⁷ is S, R, K, L, Y, A, G, P, Q, or absent; X⁸ is N, K, A, R, P, G, or absent; X⁹ is N, G, K, L, H, or absent; X¹⁰ is R, L, A, K, T, or absent; X¹¹ is R, G, A, K, S or absent; X¹² is P, M, or absent; X¹³ is L, Q, W, A, Y, G, R, K, or absent; X¹⁴ is G, P, N, M, Nle, or absent; X¹⁵ is L, R, T, or absent; X¹⁶ is A, L, V, S, Q, P, T, or absent; X¹⁷ is G, E, M, or D; and X¹⁸ s is S, P, or absent X¹⁹ is absent, —NH-T¹ or —(C═O)-T¹, wherein T¹ is a terminal group; * represents either the N-terminus of the cleavable peptide or a point of attachment to an additional group A¹, and ** represents the C-terminus of the cleavable peptide or a point of attachment to an additional group A¹; wherein one or more side chains of any of amino acids X¹—X¹⁹ can be directly or indirectly covalently bonded to one or more additional groups A¹; wherein the cleavable peptide comprises at least two protease cleavage sites, wherein each of the at least two protease cleavage sites is cleaved by a different protease; and wherein each additional group A¹ can be the same additional group A¹ attached at multiple locations of the cleavable peptide or can be different additional groups A¹.

In an aspect provided herein is a cleavable peptide attached a first additional group A¹, wherein the cleavable peptide comprises an amino acid sequence according to any one of SEQ ID NOs: 301-381 with up to 3 amino acid substitutions, wherein the cleavable peptide is optionally attached to a second additional group A¹.

In an aspect further provided herein is a cleavable peptide comprising a peptide of formula G³, wherein G³ has a structure: *—X^{A1}—X^{A2}—X^{A3}—X^{A4}—X^{A5}—X^{A6}—X^{A7}—X^{A8}—X^{A9}—X^{A10}—**; wherein X^{A1} is T, ornithine (Orn), H, S, R, or K; X^{A2} is S, D, E, T, A, N, G, or Q; X^{A3} is Orn, H, R, K, or absent; X^{A4} is Or, H, R, or K; X^{A5} is L, G, I, Nle, M, V, or P; X^{A6} is Y, F, V, I, Nle, M, Q, A, or L; X^{A7} is S, D, E, T, M, P, Q, N, or G; X^{A8} is Y, F, L, P, or absent; X^{A9} is Y, F, T, L, I, Nle, M, Q, V, A, or S; X^{A10} is Q, E, S, T, N, D, or G; wherein * represents either the N-terminus of the cleavable peptide or a point of attachment to an additional group A¹; and ** represents the C-terminus of the cleavable peptide or a point of attachment to an additional group A¹; wherein each additional group A¹ can be the same additional group A¹ attached at multiple locations of the cleavable peptide or can be different additional groups A¹. In some embodiments, the cleavable peptide comprises at least two protease cleavage sites. In some embodiments, X^{A1} is T, Orn, H, S, R, or K; X^{A2} is A, N, G, or Q; X^{A3} is Orn, H, R, K, or absent; X^{A4} is Orn, H, R, or K; X^{A5} is L, G, I, Nle, M, V, or P; X^{A6} is V, I, Nle, M, Q, A, or L; X^{A7} is M, P, Q, N, or G; X^{A8} is L, P, or absent; X^{A9} is, T, L, I, Nle, M, Q, V, A, or S; and X^{A10} is N, D, or G. In some embodiments, X^{A1} is S, R, or K; X^{A2} is G or Q; X^{A3} is R, K, or absent; X^{A4} is R or K; X^{A5} is V or P; X^{A6} is A or L; X^{A7} is N or G; X^{A8} is L or P; X^{A9} is V, A, or S; and X^{A10} is D or G. In some embodiments, X^{A1} is S or R; X^{A2} is G; X^{A3} is R or absent; X^{A4} is R, X^{A5} is V; X^{A6} is A; X^{A7} is N; X^{A8} is L; X^{A9} is V; and X^{A10} is G. In some embodiments, X^{A5} is V; X^{A6} is A; X^{A7} is N; X^{A8} is L; X^{A9} is V; and X^{A10} is G. In some embodiments, X^{A1} is S, R, or K; X^{A2} is G or Q; X^{A3} is R, K, or absent; and X^{A4} is R or K. In some embodiments, X^{A1} is S or R; X^{A2} is G; X^{A3} is R or absent; and X^{A4} is R. In some embodiments, X^{A5} is V or P; X^{A6} is A or L; X^{A7} is N or G; X^{A8} is L or P; X^{A9} is V, A, or S; and X^{A10} is D or G. In some embodiments, the cleavable peptide does not comprise the sequence PLGLAG (SEQ ID NO: 129).

In some embodiments, each of the additional groups A¹ is independently selected from a polypeptide, a nucleic acid, a polysaccharide, a lipid, an antibody, an organic biopolymer, a chemical polymer, a drug, a nanoparticle, a dye, or a bio-organic molecule. In some embodiments, the cleavable peptide comprises two different additional groups A¹. In some embodiments, cleavage of any one of the at least two protease cleavage sites causes the two different additional groups A¹ to no longer be covalently linked. In some embodiments, the cleavable peptide comprises an additional group A¹ attached at two locations of the cleavable peptide to form a cyclic structure. In some embodiments, cleavage of any one of the at least two protease cleavage sites breaks the cyclic structure. In some embodiments, each additional group A¹ is optionally connected to the cleavable peptide by a linker.

In some embodiments, the cleavable peptide is cleavable by at least two proteases selected from a kallikrein, thrombin, chymase, carboxyprotease A, and elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease.

In some embodiments, the cleavable peptide comprises one or more of the subsequences SSR, SGG, SGR, RQR, RGR GKS, RKA, KAG, PAS, PAG, LAG, LVG, LVD, LQG, LLE, PQP, PWG, PLN, LSG, LAM, MRA, MTA, LPG, LTP, LTG, TGP, FTA, MKG, LPG, GEE, or VAN. In some embodiments, the cleavable peptide comprises one or more of the subsequences SSRV (SEQ ID NO: 401), SSRG (SEQ ID NO: 402), SGRV (SEQ ID NO: 403), RKAG (SEQ ID NO: 404), RQRR (SEQ ID NO: 405), RGRR (SEQ ID NO: 406), RGRK (SEQ ID NO: 407), RQRK (SEQ ID NO: 408), GKSA (SEQ ID NO: 409), MLQG (SEQ ID NO: 410), Nle-LQG, PASN (SEQ ID NO: 412), or PLVD (SEQ ID NO: 413). In some embodiments, the cleavable peptide comprises one or more of the subsequences SGRVL (SEQ ID NO: 423), RQRRS (SEQ ID NO: 415), RGRKS (SEQ ID NO: 416), RGRKP (SEQ ID NO: 417), SGRVA (SEQ ID NO: 418), SGRVY (SEQ ID NO: 419), SGGPG (SEQ ID NO: 420), AGLVG (SEQ ID NO: 421), or ANLVG (SEQ ID NO: 422). In some embodiments, the cleavable peptide comprises one or more of the sequences PLG, PAN, PLN, PWG, PQP, or PAG. In some embodiments, the cleavable peptide comprises one or more of the sequences PLGLAG (SEQ ID NO: 129), PQPLVD (SEQ ID NO: 425), PWGLLE (SEQ ID NO: 426), PAGLVG (SEQ ID NO: 427), PANLVG (SEQ ID NO: 428), PLNLSG (SEQ ID NO: 429), AGLVG (SEQ ID NO: 430), ANLVG (SEQ ID NO: 431), PANLAG (SEQ ID NO: 432), GEEGEE (SEQ ID NO: 433), MKGLPG (SEQ ID NO: 434), RGRKSP (SEQ ID NO: 435), RGRKPY (SEQ ID NO: 436), RGRKPQ (SEQ ID NO: 437), PAGLTP (SEQ ID NO: 438), or PAGLTG (SEQ ID NO: 439). In some embodiments, the cleavable peptide comprises a sequence LAG, LVG, or LQG. In some embodiments, the cleavable peptide comprises a subsequence EAGRSANHT (SEQ ID NO: 440). In some embodiments, the cleavable peptide comprises a sequence SSRG (SEQ ID NO: 402), SSRA (SEQ ID NO: 414), SGRV (SEQ ID NO: 403), or SSRV (SEQ ID NO: 401). In some embodiments, the cleavable peptide comprises a sequence RQRR (SEQ ID NO: 405), RGRK (SEQ ID NO: 407), or RGRR (SEQ ID NO: 406). In some embodiments, the cleavable peptide comprises a sequence RQRRS (SEQ ID NO: 415) or RQRRV (SEQ ID NO: 424). In some embodiments, the cleavable peptide comprises one or more of the peptides: SSRGPASNRRLPLGLAG (SEQ ID NO: 301), SSRAVFRKNLGPLGLAG (SEQ ID NO: 302), SGRVLRKAGPQPLVD (SEQ ID NO: 303), SGRVLTLRKAPWGLLE (SEQ ID NO: 304), SGRVLGPAGLVG (SEQ ID NO: 305), SGRVLGPANLVG (SEQ ID NO: 306), SGRVLPAGLVG (SEQ ID NO: 307), SGRVLGPLNLSG (SEQ ID NO: 308), RQRRSAPLGLAG (SEQ ID NO: 309), RQRRSPLGLAG (SEQ ID NO: 310), SGRVLTLRKAGPAGLVG (SEQ ID NO: 311), SGRVLTLRKAGPANLVG (SEQ ID NO: 312), SGRVLRKAGPAGLVG (SEQ ID NO: 313), SGRVLRKAGPANLVG (SEQ ID NO: 314), SGRVLPANLVG (SEQ ID NO: 315), SGRVAGLVG (SEQ ID NO: 316), SGRVANLVG (SEQ ID NO: 317), PASNRRLPLGLAG (SEQ ID NO: 318), SSRVFRKPANLAG (SEQ ID NO: 319), SGRVLTLRKAALPLAM (SEQ ID NO: 320), SSRGRRGPYMLQG (SEQ ID NO: 321), SSRGPYMLQG (SEQ ID NO: 322), SGRVLPLGMRA (SEQ ID NO: 323), SGRVLPYAMTA (SEQ ID NO: 324), RQRRS-Nle-PLGLAG (SEQ ID NO: 325), KWGKSAPLGLAG (SEQ ID NO: 326), RYGKSAPLGLAG (SEQ ID NO: 327), RQRRSAAPLGLAG (SEQ ID NO: 328), RQRRSVVGG (SEQ ID NO: 329), SPLGLAGS (SEQ ID NO: 330), RGRKVANLVG (SEQ ID NO: 331), RQRKVANLVG (SEQ ID NO: 332), RGRRVANLVG (SEQ ID NO: 333), RGRKSPANLVG (SEQ ID NO: 334), RGRKPYMLQG (SEQ ID NO: 335), RGRKPY-Nle-LQG (SEQ ID NO: 336), RGRKSPYMLQG (SEQ ID NO: 337), RGRKSPY-Nle-LQG (SEQ ID NO: 338), RGRKPQPLVD (SEQ ID NO: 339), RGRKSPQPLVD (SEQ ID NO: 340), RGRKSQPLVD (SEQ ID NO: 341), SGRVAPYMLQG (SEQ ID NO: 342), SGRVAPY-Nle-LQG (SEQ ID NO: 343), SGRVYMLQG (SEQ ID NO: 344), SGRVY-Nle-LQG (SEQ ID NO: 345), SGRVAPQPLVD (SEQ ID NO: 346), SGRVQPLVD (SEQ ID NO: 347), RGRRGP (SEQ ID NO: 348), FTARSAPLGLAG (SEQ ID NO: 216), FTAKSPLGLAG (SEQ ID NO: 218), SSRGPLGLAG (SEQ ID NO: 237), SSRGPRGLAG (SEQ ID NO: 238), SGGPGPAGMKGLPGS (SEQ ID NO: 101), MKGLPGS (SEQ ID NO: 441), GEEGEEPLGLAG (SEQ ID NO: 256), PLGLAG (SEQ ID NO: 129), LAG, KPLGLAG (SEQ ID NO: 442), KKPLGLAG (SEQ ID NO: 443), GPLGLAG (SEQ ID NO: 257), GEAGRSANHTPAGLTP (SEQ ID NO: 444), or EAGRSANHTPAGLTGP (SEQ ID NO: 258). In some embodiments, the cleavable peptide comprises one or more of the peptides: RGRRPLGLAG (SEQ ID NO: 349), RGRRVANPLGLAGSG (SEQ ID NO: 350), RGRRPLGLAGGSG (SEQ ID NO: 351), RGRRHSSKLQ (SEQ ID NO: 352), SGRVANPLGGSG (SEQ ID NO: 353), SGRVANYFGKL (SEQ ID NO: 354), RGRRVANYFGKL (SEQ ID NO: 355), SGRPLGYFGKL (SEQ ID NO: 356), RGRRPLGYFGKL (SEQ ID NO: 357), RGRRVANPLGYFGKL (SEQ ID NO: 358), RGRRSGRAANLVRPLGYFGKL (SEQ ID NO: 359), RGRRAANLVRPLGYFGKL (SEQ ID NO: 360), HSSKLQYFGKL (SEQ ID NO: 361), RGRRHSSKLQPLGYFGKL (SEQ ID NO: 362), SGRHSSKLQPLGYFGKL (SEQ ID NO: 363), GSGSGSGS (SEQ ID NO: 364), SSLYSSPG (SEQ ID NO: 365), SSLQSSPG (SEQ ID NO: 366), SQYQSSPG (SEQ ID NO: 367), SQLYSSPG (SEQ ID NO: 368), SSQYSSPG (SEQ ID NO: 369), ISQYSSAT (SEQ ID NO: 370), KLYSSKQ (SEQ ID NO: 371), KLFSSKQ (SEQ ID NO: 372), RRLHYSL (SEQ ID NO: 373), RRLNYSL (SEQ ID NO: 374), RSSYRSL (SEQ ID NO: 375), RSSYYSL (SEQ ID NO: 376), KSKQHSL (SEQ ID NO: 377), HSSKLQL (SEQ ID NO: 378), GSSYYSGA (SEQ ID NO: 379), GSSVYSGR (SEQ ID NO: 380), or SS-Nle-YSSAG (SEQ ID NO: 381). In some embodiments, the cleavable peptide comprises an amino acid sequence set forth in Table 2 or Table 3A. In some embodiments, the cleavable peptides comprises an amino acid sequence set forth in Table 3B.

In some embodiments, the cleavable peptide is incorporated into the amino acid sequence of a protein. In some embodiments, the protein is a recombinant protein. In some embodiments, the protein is a fusion protein. In some embodiments, the cleavable peptide is positioned between domains of the fusion protein. In some embodiments, the cleavable peptide is attached to a side chain of an amino acid of a protein. In some embodiments, the protein is an activatable protein.

In an aspect, described herein are activatable proteins comprising a first protein molecule and a first cleavable peptide, wherein the cleavable peptide is attached to the protein at a first site. In some embodiments, the first site is a side chain of an amino acid of the protein. In some embodiments, the first site is the N-terminal amine or the C-terminal carboxyl of the protein (optionally via a linker, or directly attached as in, for example, a fusion protein). In some embodiments, the cleavable peptide is incorporated into an internal site of the protein (e.g, between different domains of the protein, such as between two genes combined in a fusion protein). In some embodiments, the activatable protein has several points of attachment on the amino acid side chain. In some embodiments, the cleavable peptide is attached to side chain of an amino acid residue in the protein via a covalent bond. In some embodiments, the cleavable peptide is attached to the protein at multiple points of attachment (e.g., at the N-terminal amine and a side chain of an amino acid residue, at the C-terminal carboxyl and a side chain of an amino acid residue, or at the side chains of two different amino acid residues). In some embodiments, site specific cleavage at the cleavable peptide modulates the activity of the activatable protein.

In another aspect, described herein the activatable proteins comprise more than one protein molecules attached to the cleavable peptides, wherein the cleavable peptides are attached to the one or more protein molecules via amino acid side chains.

In some embodiments, the activatable protein comprises a protease-cleavable group, wherein the protein has the formula:
P¹-L¹-(G¹)_{q}, wherein P¹ is a first protein having a first site, which is an amino acid side chain; L¹ is a covalent bond between G¹ and P¹ or a divalent linker bound to P¹ at the first site and to G¹; and q is an integer between 1 to 50; and each G¹ independently comprises a protease-cleavable peptide.

In one embodiment, the protease-cleavable peptide G¹ has the formula:

*—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—*
wherein:
X¹ is S, R, P, K, F, G, or absent;
X² is S, G, Q, A, W, Y, T, E or absent;
X³ is R, S, G, A, E, or absent;
X⁴ is G, A, V, R, N, K, P, or absent;
X⁵ is P, V, L, S, F, R, A, Y, Q, G, E or absent;
X⁶ is A, F, R, T, G, P, Nle, V, Y, Q, E, S, or absent;
X⁷ is S, R, K, L, Y, A, G, P, Q, or absent;
X⁸ is N, K, A, R, P, G, or absent;
X⁹ is N, G, K, L, H, or absent;
X¹⁰ is R, L, A, K, T, or absent;
X¹¹ is R, G, A, K, S or absent;
X¹² is P, M, or absent;
X¹³ is L, Q, W, A, Y, G, R, K, or absent;
X¹⁴ is G, P, N, M, Nle, or absent;
X¹⁵ is L, R, T, or absent;
X¹⁶ is A, L, V, S, Q, P, T, or absent;
X¹⁷ is G, E, M, or D; and
X¹⁸ is S, P, or absent;
X¹⁹ is absent, —NH-T¹ or —(C═O)-T¹,
wherein T¹ is a terminal group, a group B¹, wherein B¹ is a blocking moiety, or a linker L², wherein L² is covalently bonded to the protein P¹ at a second site separate from the first site to form a cyclic protein with P¹; * represents either the C- or N-terminus of the cleavable peptide and ** represents the other of the C- or N-terminus of the cleavable peptide; wherein one or more side chains of any of amino acids X¹—X¹⁹ can be directly or indirectly covalently bonded to one or more groups B², wherein B² is a blocking moiety.

In some embodiments, the first cleavable peptide comprises at least three amino acids. In some embodiments, the cleavable peptide comprises one or more of the subsequences SSR, SGG, SGR, RQR, RGR GKS, RKA, KAG, PAS, PAG, LAG, LVG, LVD, LQG, LLE, PQP, PWG, PLN, LSG, LAM, MRA, MTA, LPG, LTP, LTG, TGP, FTA, MKG, LPG, GEE, or VAN. In some embodiments, the cleavable peptide comprises one or more of the subsequences SSRV (SEQ ID NO: 401), SSRG (SEQ ID NO: 402), SGRV (SEQ ID NO: 403), RKAG (SEQ ID NO: 404), RQRR (SEQ ID NO: 405), RGRR (SEQ ID NO: 406), RGRK (SEQ ID NO: 407), RQRK (SEQ ID NO: 408), GKSA (SEQ ID NO: 409), MLQG (SEQ ID NO: 410), Nle-LQG, PASN (SEQ ID NO: 412), or PLVD (SEQ ID NO: 413). In some embodiments, the cleavable peptide comprises at least five amino acids. In some embodiments, the cleavable peptide comprises one or more of the subsequences SGRVL (SEQ ID NO: 423), RQRRS (SEQ ID NO: 415), RGRKS (SEQ ID NO: 416), RGRKP (SEQ ID NO: 417), SGRVA (SEQ ID NO: 418), SGRVY (SEQ ID NO: 419), SGGPG (SEQ ID NO: 420), AGLVG (SEQ ID NO: 421), or ANLVG (SEQ ID NO: 422).

In some other embodiments, the cleavable peptide comprises at least six amino acids. In some embodiments, the cleavable peptide comprises one or more of the sequences PLG, PAN, PLN, PWG, PQP, or PAG. In some embodiments, the cleavable peptide comprises one or more of the sequences PLGLAG (SEQ ID NO: 129), PQPLVD (SEQ ID NO: 425), PWGLLE (SEQ ID NO: 426), PAGLVG (SEQ ID NO: 427), PANLVG (SEQ ID NO: 428), PLNLSG (SEQ ID NO: 429), AGLVG (SEQ ID NO: 430), ANLVG (SEQ ID NO: 431), PANLAG (SEQ ID NO: 432), GEEGEE (SEQ ID NO: 433), MKGLPG (SEQ ID NO: 434), RGRKSP (SEQ ID NO: 435), RGRKPY (SEQ ID NO: 436), RGRKPQ (SEQ ID NO: 437), PAGLTP (SEQ ID NO: 438), or PAGLTG (SEQ ID NO: 439).

In some embodiments, the cleavable peptide comprises a sequence LAG, LVG, or LQG. In some embodiments, the cleavable peptide comprises a subsequence EAGRSANHT (SEQ ID NO: 440). In some other embodiments, the cleavable peptide comprises a sequence SSRG (SEQ ID NO: 402), SSRA (SEQ ID NO: 414), SGRV (SEQ ID NO: 403), or SSRV (SEQ ID NO: 401). In some embodiments, the cleavable peptide comprises a sequence RQRR (SEQ ID NO: 405), RGRK (SEQ ID NO: 407), or RGRR (SEQ ID NO: 406). In some embodiments, the cleavable peptide comprises a sequence RQRRS (SEQ ID NO: 415) or RQRRV (SEQ ID NO: 424).

In some embodiments the cleavable peptide comprises one or more of the peptides: SSRGPASNRRLPLGLAG (SEQ ID NO: 301), SSRAVFRKNLGPLGLAG (SEQ ID NO: 302), SGRVLRKAGPQPLVD (SEQ ID NO: 303), SGRVLTLRKAPWGLLE (SEQ ID NO: 304), SGRVLGPAGLVG (SEQ ID NO: 305), SGRVLGPANLVG (SEQ ID NO: 306), SGRVLPAGLVG (SEQ ID NO: 307), SGRVLGPLNLSG (SEQ ID NO: 308), RQRRSAPLGLAG (SEQ ID NO: 309), RQRRSPLGLAG (SEQ ID NO: 310), SGRVLTLRKAGPAGLVG (SEQ ID NO: 311), SGRVLTLRKAGPANLVG (SEQ ID NO: 312), SGRVLRKAGPAGLVG (SEQ ID NO: 313), SGRVLRKAGPANLVG (SEQ ID NO: 314), SGRVLPANLVG (SEQ ID NO: 315), SGRVAGLVG (SEQ ID NO: 316), SGRVANLVG (SEQ ID NO: 317), PASNRRLPLGLAG (SEQ ID NO: 318), SSRVFRKPANLAG (SEQ ID NO: 319), SGRVLTLRKAALPLAM (SEQ ID NO: 320), SSRGRRGPYMLQG (SEQ ID NO: 321), SSRGPYMLQG (SEQ ID NO: 322), SGRVLPLGMRA (SEQ ID NO: 323), SGRVLPYAMTA (SEQ ID NO: 324), RQRRS-Nle-PLGLAG (SEQ ID NO: 325), KWGKSAPLGLAG (SEQ ID NO: 326), RYGKSAPLGLAG (SEQ ID NO: 327), RQRRSAAPLGLAG (SEQ ID NO: 328), RQRRSVVGG (SEQ ID NO: 329), SPLGLAGS (SEQ ID NO: 330), RGRKVANLVG (SEQ ID NO: 331), RQRKVANLVG (SEQ ID NO: 332), RGRRVANLVG (SEQ ID NO: 333), RGRKSPANLVG (SEQ ID NO: 334), RGRKPYMLQG (SEQ ID NO: 335), RGRKPY-Nle-LQG (SEQ ID NO: 336), RGRKSPYMLQG (SEQ ID NO: 337), RGRKSPY-Nle-LQG (SEQ ID NO: 338), RGRKPQPLVD (SEQ ID NO: 339), RGRKSPQPLVD (SEQ ID NO: 340), RGRKSQPLVD (SEQ ID NO: 341), SGRVAPYMLQG (SEQ ID NO: 342), SGRVAPY-Nle-LQG (SEQ ID NO: 343), SGRVYMLQG (SEQ ID NO: 344), SGRVY-Nle-LQG (SEQ ID NO: 345), SGRVAPQPLVD (SEQ ID NO: 346), SGRVQPLVD (SEQ ID NO: 347), RGRRGP (SEQ ID NO: 348), FTARSAPLGLAG (SEQ ID NO: 216), FTAKSPLGLAG (SEQ ID NO: 218), SSRGPLGLAG (SEQ ID NO: 237), SSRGPRGLAG (SEQ ID NO: 238), SGGPGPAGMKGLPGS (SEQ ID NO: 101), MKGLPGS (SEQ ID NO: 441), GEEGEEPLGLAG (SEQ ID NO: 256), PLGLAG (SEQ ID NO: 129), LAG, KPLGLAG (SEQ ID NO: 442), KKPLGLAG (SEQ ID NO: 443), GPLGLAG (SEQ ID NO: 257), GEAGRSANHTPAGLTP (SEQ ID NO: 444), or EAGRSANHTPAGLTGP (SEQ ID NO: 258). In some embodiments, the cleavable peptide comprises one or more of the peptides: RGRRPLGLAG (SEQ ID NO: 349), RGRRVANPLGLAGSG (SEQ ID NO: 350), RGRRPLGLAGGSG (SEQ ID NO: 351), RGRRHSSKLQ (SEQ ID NO: 352), SGRVANPLGGSG (SEQ ID NO: 353), SGRVANYFGKL (SEQ ID NO: 354), RGRRVANYFGKL (SEQ ID NO: 355), SGRPLGYFGKL (SEQ ID NO: 356), RGRRPLGYFGKL (SEQ ID NO: 357), RGRRVANPLGYFGKL (SEQ ID NO: 358), RGRRSGRAANLVRPLGYFGKL (SEQ ID NO: 359), RGRRAANLVRPLGYFGKL (SEQ ID NO: 360), HSSKLQYFGKL (SEQ ID NO: 361), RGRRHSSKLQPLGYFGKL (SEQ ID NO: 362), SGRHSSKLQPLGYFGKL (SEQ ID NO: 363), GSGSGSGS (SEQ ID NO: 364), SSLYSSPG (SEQ ID NO: 365), SSLQSSPG (SEQ ID NO: 366), SQYQSSPG (SEQ ID NO: 367), SQLYSSPG (SEQ ID NO: 368), SSQYSSPG (SEQ ID NO: 369), ISQYSSAT (SEQ ID NO: 370), KLYSSKQ (SEQ ID NO: 371), KLFSSKQ (SEQ ID NO: 372), RRLHYSL (SEQ ID NO: 373), RRLNYSL (SEQ ID NO: 374), RSSYRSL (SEQ ID NO: 375), RSSYYSL (SEQ ID NO: 376), KSKQHSL (SEQ ID NO: 377), HSSKLQL (SEQ ID NO: 378), GSSYYSGA (SEQ ID NO: 379), GSSVYSGR (SEQ ID NO: 380), or SS-Nle-YSSAG (SEQ ID NO: 381).

In some embodiments, the terminal group T¹ is one or more branched or unbranched C₁-C₁₂ alkyl. In some other embodiments, T¹ is —CH₃, —CH₂CH₃, isopropyl, n-propyl, n-butyl, s-butyl, or t-butyl.

In some embodiments, the terminal group T¹ is one or more blocking moieties, (for example, B¹, B², etc.) The blocking moiety can be any molecule that blocks a portion of the first protein molecule such that the first protein molecule is rendered at least partially inactive. In some embodiments, the activatable protein comprises multiple blocking moieties, B¹ to B⁵⁰.

In some embodiments, at least one blocking moiety is a second protein molecule or another macromolecule including but not limited to nucleic acids, polysaccharides, proteins, lipids, antibodies, organic biopolymers, chemical polymers, peptides, or bio-organic molecules. In some embodiments, the second protein is one which binds to or otherwise interacts with the first protein and blocks the interaction of the first protein with at least one binding partner of the first protein (e.g., a cognate receptor of the first protein). In some embodiments, the second protein is a dummy receptor of the first protein (i.e., the second protein mimics the natural receptor of the first protein, thereby blocking interaction of the first protein with the natural receptor when the dummy receptor is attached to the protein through the cleavable peptide). In some embodiments, the second protein is a bulky protein whose presence, when attached to the first protein by the cleavable peptide, at least partially blocks the interaction of the first protein with at least one binding partner of the first protein (e.g., a cognate receptor of the first protein).

In some embodiments, the blocking moiety is a linker L², wherein L² is covalently bonded to the first protein molecule at a second site separate from the first site to form a cyclic activatable protein. In some embodiments, L² comprises a straight or branched PEGₚ (polyethylene glycol), wherein p is an integer from 1 to 50 and represents the number of ethylene glycol monomer subunits in PEGₚ. In some embodiments, the first cleavable peptide linker comprises multiple blocking moieties.

In some embodiments, the activatable protein comprises a cyclic protein of formula:

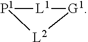

In some embodiments, L² is a branched or unbranched polyethylene glycol linker.

In some embodiments, the one or more activatable protein molecules are natural and/or synthetic. In some embodiments, the first protein molecule comprises a naturally-occurring or synthetic cytokine. In some embodiments, the first protein molecule comprises a naturally-occurring or synthetic interleukin. In some embodiments, the first protein molecule comprises a synthetic interleukin. In some embodiments, the first protein molecule comprises a synthetic interleukin and a second peptide. In some embodiments, the first protein molecule comprises a synthetic interleukin and an antibody or antibody fragment. In some embodiments, the antibody or antibody fragment binds to a checkpoint inhibitor.

In one embodiment, the first protein molecule is a synthetic IL-2, wherein a cleavable peptide is linked to the side chain of synthetic IL-2. In some embodiments, cleavage of the first cleavable peptide enhances the binding of the synthetic IL-2 protein to one or more IL-2 receptor subunits.

In another embodiment, described herein are cleavable peptide moieties that are attached to the side chain of an amino acid in a peptide.

In an embodiment, described herein is a composition comprising the activatable protein and one or more pharmaceutically acceptable solvents.

In yet another embodiment, described herein is a method of treatment comprising administering to a patient in need thereof the activatable protein or the pharmaceutical composition comprising the activatable protein.

In some embodiments, the invention is useful in treating a disease (e.g., a cancer) with the protein by conditionally modifying the activity of the protein. In some embodiments, activity of the protein is conditionally modified when the protein encounters an environment related to a non-disease state, or an environment related to a disease state (e.g., a tumor microenvironment). In some embodiments, environmental factors (e.g., a protease) causes cleavage of the cleavable peptide, thus modifying the activity of the protein. The invention has the advantage of treating the diseased tissue or cell while leaving normal tissues unaffected.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1 shows the cleavage efficiency of tested cleavable peptides with different enzymes.

FIG. 2A shows the cleavage kinetics of selected peptides with different proteases.

FIG. 2B shows the cleavage kinetics of selected peptides with different proteases.

FIG. 3 shows the cleavage efficiency of selected cleavable peptides at pH 6.5 and pH 7.5.

FIG. 4 shows the cleavage kinetics of selected cleavable peptides with different proteases.

FIG. 5 shows the cleavage kinetics of selected cleavable peptides with different MMPs.

FIG. 6 shows the cleavage kinetics of selected cleavable peptides with kallikrein-3.

### DETAILED DESCRIPTION

### Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein, the term “amino acid” refers to either natural and/or unnatural or synthetic amino acids, including but not limited to both the D or L optical isomers, and amino acid analogs and peptidomimetics. Standard single or three letter codes may be used to designate amino acids. In preferred instances, any reference to an amino acid herein refers to the L optical isomer, in particular in the context of cleavable peptides.

In certain embodiments, amino acid substitutions herein relative to a reference sequence are described as “conservative” amino acid substitutions. As used herein, conservative amino acid substitutions refer to the replacement of a given amino acid by a residue having similar physiochemical characteristics, e.g., substituting one aliphatic residue for another (such as Ile, Val, Leu, Nle, or Ala for one another), or substitution of one polar residue for another (such as between Lys and Arg; Glu and Asp; or Gln and Asn, and in some instances preferably retaining the same charge). Polypeptides comprising conservative amino acid substitutions can be tested in any one of the assays described herein to confirm that a desired activity, e.g. cleavage by a protease as described herein, is maintained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), lie (I), Pro (P), Phe (F), Trp (W), Met (M), norleucine (Nle); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, He; (2) neutral hydrophilic: Cys, Ser, Thr, Asn, Gin; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val or into Nle; Leu into Ile or into Val or into Nle; Lys into Arg or into Gln or into Glu; Met into Leu, into Tyr, or into Ile; Nle into Ile, or into Leu, or into Val; Phe into Met, or into Nle, or into Leu, or into Tyr, or into Val, or into Ile, or into Leu; Ser into Thr; Thr into Ser; Trp into Tyr or into Phe; and/or Tyr into Trp or into Phe.

The term “non-naturally occurring,” or “synthetic” as applied to sequences or polypeptides or amino acids, and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

As used herein in the context of the structure of a polypeptide, “N-terminus” (or “amino terminus”) and “C-terminus” (or “carboxyl terminus”) refer to the extreme amino and carboxyl ends of the polypeptide, respectively.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of, for example, cleavable peptides described herein. As used herein, “attached” or “covalently attached” means that the group is tethered to the indicated residue, and such tethering can include a linking group (i.e., a linker). Thus, for a group “attached” or “covalently attached” to a residue, it is expressly contemplated that such linking groups are also encompassed.

“Activity” as applied to form(s) of a composition or the activatable protein provided herein, refers to an action or effect, including but not limited to receptor binding, antagonist activity, agonist activity, a cellular or physiologic response, cell lysis, cell death, or an effect generally known in the art for the effector component of the composition, whether measured by an in vitro, ex vivo or in vivo assay or a clinical effect.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 can comprise 1 to 10, 1 to 20, 1 to 30, and 1 to 40 in one direction, or 50 to 40, 50 to 30, 50 to 20, and 50 to 10 in the other direction.

The term “subject” and/or “patient” refers to an animal which is the object of treatment, observation, or experiment. By way of example only, a “subject” and/or “patient” includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities attached to, embedded in, or appended to a molecule.

The N-terminus or C-terminus of a protein can refer to the first and last natural amino acids present, respectively, in the natural version of the protein, or a corresponding amino acid substituted at that position.

### Cleavable Peptides

In one aspect, provided herein, are novel cleavable peptides. In some embodiments, the cleavable peptides are cleavable by multiple proteases. In some embodiments, the cleavable peptides are cleavable by multiple proteases which are upregulated or associated with upregulation in or near a tumor or tumor microenvironment. In some embodiments, the cleavable peptides provided herein are selectively cleaved in or near a tumor or tumor microenvironment. In some embodiments, the cleavable peptides provided herein are efficiently cleaved in or near a tumor or tumor microenvironment.

Exemplary cleavable peptides, along with specific proteases capable of cleaving the said cleavable peptides, are provided in Table 1 below. Such sequences, or portions thereof, can, in some embodiments, be incorporated into a cleavable peptide of the instant invention.

TABLE 1
Exemplary cleavage sites

| SEQ | Cleavable | |
|---|---|---|
| ID | Peptide | |
| NO: | Sequence | Protease |
| 101 | SGGPGPAGMKGLPGS | MMP9 |
| 102 | EAGRSANHEPLGLVAT | MMP2-7-9-14 + |
| | | matriptase + |
| | | uPA + Legumain |
| 103 | PQASTGRSGG | MMP9 + |
| | | matriptase + |
| | | uPA |
| 104 | PQGSTGRAAG | MMP9 + |
| | | matriptase + |
| | | uPA |
| 105 | PPASSGRAGG | MMP9 + |
| | | matriptase + |
| | | uPA |
| 106 | RSGVPLSLYSGSGGGK | MMP7-9-10 |
| 107 | RSGMPYDLYHPSGK | MMP7-9-10 |
| 108 | RGPDSGGFMLTSGK | MMP7-9-10 |
| 109 | RGSGHEQLTVSGGSK | MMP7-9-10 |
| 110 | RSGRAAAVKSPSGK | MMP7-9-10 |
| 111 | RGSGISSGLLSGRSDNHSGK | MMP7-9-10 |
| 112 | RGDLLAVVAASGGK | MMP7-9-10 |
| 113 | RGGISSGLLSGRSGK | MMP7-9-10 |
| 114 | SGGGKKLADEPEGGS | Meprin A/B |
| 115 | GGGKFLADEPEGG | Meprin A/B |
| | | (High Efficiency) |
| 116 | ARLQSAAP | Cathepsin S, K, L |
| 117 | ARLQSAAPAGLKGA | Cathepsin S + |
| | | MMP9 + Meprin A |
| 118 | GSGGPGPAGMHGLPGGS | MMP9 |
| | | (High Efficiency) |
| 119 | GGGSHTGRSAYFGGGS | uPA |
| 120 | SGGPGPAGLKGAPGS | MMP9-2 |
| 121 | VPLSLYSG | MMP2-7-9 |
| 122 | SGLLSGRSDNH | uPA + |
| | | Matriptase + |
| | | legumain |
| 123 | SGRSDNIGGGS | uPA |
| 124 | LQESLRSKESGRSDI | MMP-14 + uPA |
| 125 | ISSGLLSGRSDNH | uPA |
| 126 | LSGRSDDH | uPA |
| 127 | ISSGLLSGRSDQH | uPA |
| 128 | ISSGLLSGRSDNI | uPA |
| 129 | PLGLAG | MMP2-7-9 |
| 130 | GPAGMKGL | MMP |
| 131 | LSGRSDQH | MMP |
| 132 | LSGRSDNI | MMP |
| 133 | ISSGLLSGRSDNH | MMP |
| 134 | GPLGVRG | MMP |
| 135 | GPLGLAR | MMP |
| 136 | GPAALVGA | MMP |
| 137 | GPAALIGG | MMP |
| 138 | GPLNLVGR | MMP |
| 139 | GPAGLVAD | MMP |
| 140 | GPANLVAP | MMP |
| 141 | VPLSLYSG | MMP |
| 142 | SGESPAYYTA | MMP |
| 143 | GGPRGLG | MMP |
| 144 | HSSKLQ | MMP |
| 378 | HSSKLQL | MMP |
| 146 | KRALGLPG | MMP7 |
| 147 | LEATA | MMP9 |
| 148 | GGAANLVRGG | MMP11 |
| 149 | SGRIGFLRTA | MMP14 |
| 150 | PLGLA | MMP |
| 151 | ESPAYYTA | MMP |
| 152 | RLQLKL | MMP |
| 153 | RLQLKAC | MMP |
| 154 | SGRSA | uPA |
| 155 | DAFK | uPA |
| 156 | GGGRR | uPA |
| 157 | GFLG | Lysosomal |
| | | Enzyme |
| 158 | HSSKLQEDA | Prostate |
| | | Specific |
| | | Antigen |
| 159 | LVLASSSFGY | HSV Protease |
| 160 | GVSQNYPIVG | HIV Protease |
| 161 | GVVQASCRLA | CMV Protease |
| 162 | DPRSFL | Thrombin |
| 163 | PPRSFL | Thrombin |
| 164 | DEVD | Caspase-3 |
| 165 | DEVDP | Caspase-3 |
| 166 | KGSGDVEG | Caspase-3 |
| 167 | GWEHDG | Interleukin |
| | | 1β converting |
| | | enzyme |
| 168 | EDDDDKA | Enterokinase |
| 169 | KQEQNPGST | FAP |
| 170 | GKAFRR | Kallikrein 2 |
| 171 | DAFK | Plasmin |
| 172 | DVLK | Plasmin |
| 173 | DAFK | Plasmin |
| 174 | ALLLALL | TOP |
| 175 | SGAKPRALTA | MMP A3 |
| 176 | SGLRLAAITA | MMP B49 |
| 177 | SGESLAYYTA | MMP B74 |
| 178 | SGESPAYYTA | MMP B74P |
| 179 | SGESLRYYTA | MMP B74R |
| 180 | SGRSLSRLTA | MMP C9 |
| 181 | SGRSLRRLTA | MMP C9R |
| 182 | SGAVSWLLTA | MMP A13 |
| 183 | SGAPSWLLTA | MMP A13P |
| 184 | SGAVRWLLTA | MMP A13R |
| 185 | SGANISDLTA | MMP B37 |
| 186 | SGNRYSSLTA | MMP A34 |
| 187 | SGHMHKALTA | MMP A21 |
| 188 | SGHMHKALTA | MMP A21A |

In some embodiments, a cleavable peptide provided herein conforms to a sequence of X¹—X²—X³—X⁴—X⁵—X⁶—X⁷—X⁸—X⁹—X¹⁰—X¹¹—X¹²—X¹³—X¹⁴—X¹⁵—X¹⁶-X¹⁷-X¹⁸, wherein X¹ is S, R, P, K, F, G, or absent; X² is S, G, Q, A, W, Y, T, E or absent; X³ is R, S, G, A, E, or absent; X⁴ is G, A, V, R, N, K, P, or absent; X⁵ is P, V, L, S, F, R, A, Y, Q, G, E or absent; X⁶ is A, F, R, T, G, P, Nle, V, Y, Q, E, S, or absent; X⁷ is S, R, K, L, Y, A, G, P, Q, or absent; X⁸ is N, K, A, R, P, G, or absent; X⁹ is N, G, K, L, H, or absent; X¹⁰ is R, L, A, K, T, or absent; X¹¹ is R, G, A, K, S or absent; X¹² is P, M, or absent; X¹³ is L, Q, W, A, Y, G, R, K, or absent; X¹⁴ is G, P, N, M, Nle, or absent; X¹⁵ is L, R, T, or absent; X¹⁶ is A, L, V, S, Q, P, T, or absent; X¹⁷ is G, E, M, or D; and X¹⁸ is S, P, or absent, wherein the cleavable peptide comprises at least two protease cleavage sites.

In some embodiments, a cleavable peptide provided herein is of a formula G², wherein G² has a structure

*—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—**

wherein: X¹ is S, R, P, K, F, G, or absent; X² is S, G, Q, A, W, Y, T, E or absent; X³ is R, S, G, A, E, or absent; X⁴ is G, A, V, R, N, K, P, or absent; X⁵ is P, V, L, S, F, R, A, Y, Q, G, E or absent; X⁶ is A, F, R, T, G, P, Nle, V, Y, Q, E, S, or absent; X⁷ is S, R, K, L, Y, A, G, P, Q, or absent; X⁸ is N, K, A, R, P, G, or absent; X⁹ is N, G, K, L, H, or absent; X¹⁰ is R, L, A, K, T, or absent; X¹¹ is R, G, A, K, S or absent; X¹² is P, M, or absent; X¹³ is L, Q, W, A, Y, G, R, K, or absent; X¹⁴ is G, P, N, M, Nle, or absent; X⁵ is L, R, T, or absent; X¹⁶ is A, L, V, S, Q, P, T, or absent; X⁷ is G, E, M, or D; X⁸ is S, P, or absent, and X¹⁹ is absent, —NH-T¹, or —(C═O)-T¹, wherein T¹ is a terminal group; * represents either the N-terminus of the cleavable peptide or a point of attachment to an additional group A¹; and ** represents the C-terminus of the cleavable peptide or a point of attachment to an additional group A¹; wherein one or more side chains of any of amino acids X¹—X¹⁹ can be directly or indirectly covalently bonded to one or more additional groups A¹; wherein the cleavable peptide comprises at least two protease cleavage sites, wherein each of the at least two protease cleavage sites is cleaved by a different protease; and wherein each additional group A¹ can be the same additional group A¹ attached at multiple locations of the cleavable peptide or can be different additional groups A¹.

In some embodiments, a cleavable peptide comprises a peptide having at least 80%, 90%, or 100% sequence identity to a peptide of formula G³, wherein G³ has a structure: *—X^{A1}—X^{A2}—X^{A3}—X^{A4}—X^{A5}—X^{A6}—X^{A7}—X^{A8}—X^{A9}—X^{A10}—**; wherein X^{A1} is T, ornithine (Orn), H, S, R, or K; X^{A2} is S, D, E, T, A, N, G, or Q; X^{A3} is Orn, H, R, K, or absent; X^{A4} is Orn, H, R, or K; X^{A5} is L, G, I, Nle, M, V, or P; X^{A6} is Y, F, V, I, Nle, M, Q, A, or L; X^{A7} is S, D, E, T, M, P, Q, N, or G; X^{A8} is Y, F, L, P, or absent; X^{A9} is Y, F, T, L, I, Nle, M, Q, V, A, or S; X^{A10} is Q, E, S, T, N, D, or G; wherein * represents either the N-terminus of the cleavable peptide or a point of attachment to an additional group A¹; and ** represents the C-terminus of the cleavable peptide or a point of attachment to an additional group A¹; wherein each additional group A¹ can be the same additional group A¹ attached at multiple locations of the cleavable peptide or can be different additional groups A¹. In some embodiments, the cleavable peptide comprises a peptide of formula G³. In some embodiments, the cleavable peptide comprises at least two protease cleavage sites. In some embodiments, the cleavable peptide is cleavable by an MMP and a matriptase. In some embodiments, the cleavable peptide is cleavable by an MMP between residues X^{A7} and X^{A8}. In some embodiments, the cleavable peptide is cleavable by a matriptase between residues X^{A4} and X^{A5}

Exemplary peptides conforming to SEQ ID NO: 501 described herein include, for example, at least portions of SEQ ID NOs: 315, 316, 317, 331, 332, 333, 335, 336, 339, 349, 350, 351, 353, 354, 355, 356, 357, and 358.

In some embodiments, X^{A1} is T, ornithine (Orn), H, S, R, or K. In some embodiments, X^{A1} is T, S, R, or K. In some embodiments, X^{A1} is S, R, or K. In some embodiments, X^{A1} is S or R. In some embodiments, X^{A1} is S. In some embodiments, X^{A1} is R.

In some embodiments, X^{A2} is S, D, E, T, A, N, G, or Q. In some embodiments, X^{A2} is A, N, G, or Q. In some embodiments, X^{A2} is A, N, G, or Q. In some embodiments, X^{A2} is N, G, or Q. In some embodiments, X^{A2} is G or Q. In some embodiments, X^{A2} is G. In some embodiments, X^{A2} is Q.

In some embodiments, X^{A3} is Orn, H, R, K, or absent. In some embodiments, X^{A3} is H, R, K, or absent. In some embodiments, X^{A3} is Orn, R, K, or absent. In some embodiments, X^{A3} is R, K, or absent. In some embodiments, X^{A3} is R or absent. In some embodiments, X^{A3} is R. In some embodiments, X^{A3} is absent.

In some embodiments, X^{A4} is Orn, H, R, or K. In some embodiments, X^{A4} is Orn, R, or K. In some embodiments, X^{A4} is H, R, or K. In some embodiments, X^{A4} is R or K. In some embodiments, X^{A4} is R.

In some embodiments, X^{A5} is L, G, I, Ne, M, V, or P. In some embodiments, X^{A5} is L, G, I, M, V, or P. In some embodiments, X^{A5} is L, I, M, V, or P. In some embodiments, X^{A5} is L, I, M, or V. In some embodiments, X^{A5} is V or P. In some embodiments, X^{A5} is P. In some embodiments, X^{A5} is V.

In some embodiments, X^{A6} is Y, F, V, I, Ne, M, Q, A, or L. In some embodiments, X^{A6} is Y, F, V, I, M, Q, A, or L. In some embodiments, X^{A6} is V, I, M, Q, A, or L. In some embodiments, X^{A6} is V, I, Ne, M, Q, A, or L. In some embodiments, X^{A6} is Q, A, or L. In some embodiments, X^{A6} is A or L. In some embodiments, X^{A6} is A. In some embodiments, X^{A6} is L. In some embodiments, X^{A6} is Q.

In some embodiments, X^{A7} is S, D, E, T, M, P, Q, N, or G. In some embodiments, X^{A7} is P, Q, N, or G. In some embodiments, X^{A7} is Q, N, or G. In some embodiments, X^{A7} is N or G. In some embodiments, X^{A7} is N. In some embodiments, X^{A7} is G.

In some embodiments, X^{A8} is Y, F, L, P, or absent. In some embodiments, X^{A8} is L, P, or absent. In some embodiments, X^{A8} is L or P. In some embodiments, X^{A8} is P. In some embodiments, X^{A8} is P.

In some embodiments, X^{A9} is Y, F, T, L, I, Ne, M, Q, V, A, or S. In some embodiments, X^{A9} is Y, F, T, L, I, M, Q, V, A, or S. In some embodiments, X^{A9} is T, L, I, Ne, M, Q, V, A, or S. In some embodiments, X^{A9} is T, L, I, M, Q, V, A, or S. In some embodiments, X^{A9} is Q, V, A, or S. In some embodiments, X^{A9} is V, A, or S. In some embodiments, X^{A9} is Q. In some embodiments, X^{A9} is V. In some embodiments, X^{A9} is A. In some embodiments, X^{A9} is S.

In some embodiments, X^{A10} is Q, E, S, T, N, D, or G. In some embodiments, X^{A10} is N, D, or G. In some embodiments, X^{A10} is D or G. In some embodiments, X^{A10} is D. In some embodiments, X^{A10} is G.

In some embodiments, X^{A1} is T, Orn, H, S, R, or K; X^{A2} is A, N, G, or Q; X^{A3} is Orn, H, R, K, or absent; X^{A4} is Orn, H, R, or K; X^{A5} is L, G, I, Ne, M, V, or P; X^{A6} is V, I, Ne, M, Q, A, or L; X^{A7} is M, P, Q, N, or G; X^{A8} is L, P, or absent; X^{A9} is, T, L, I, Ne, M, Q, V, A, or S; and X^{A10} is N, D, or G.

In some embodiments, X^{A1} is S, R, or K; X^{A2} is G or Q; X^{A3} is R, K, or absent; X^{A4} is R or K; X^{A5} is V or P; X^{A6} is A or L; X^{A7} is N or G; X^{A8} is L or P; X^{A9} is V, A, or S; and X^{A10} is D or G. In some embodiments, X^{A1} is S or R; X^{A2} is G; X^{A3} is R or absent; X^{A4} is R, X^{A5} is V; X^{A6} is A; X^{A7} is N; X^{A8} is L; X^{A9} is V; and X^{A10} is G.

In some embodiments, X^{A5} is V; X^{A6} is A; X^{A7} is N; X^{A8} is L; X^{A9} is V; and X^{A10} is G. In some embodiments, X^{A1} is S, R, or K; X^{A2} is G or Q; X^{A3} is R, K, or absent; and X^{A4} is R or K.

In some embodiments, X^{A1} is S or R; X^{A2} is G; X^{A3} is R or absent; and X^{A4} is R. In some embodiments, X^{A5} is V or P; X^{A6} is A or L; X^{A7} is N or G; X^{A8} is L or P; X^{A9} is V, A, or S; and X^{A10} is D or G.

In some embodiments, the cleavable peptide of SEQ ID NO: 501 does not comprise the sequence PLGLAG (SEQ ID NO: 129).

Exemplary cleavable peptides according to the instant invention are provided in Table 2 below. In Table 2 below (and elsewhere in the instant disclosure), Nle denotes a norleucine (i.e., L-norleucine) residue.

TABLE 2
Selected protease cleavage sites.

| SEQ ID NO: | Cleavable Peptide Sequence |
|---|---|
| 201 | AGDKSPLGLAG |
| 202 | AGDRSPLGLAG |
| 203 | AGDRSAPLGLAG |
| 204 | AWGRSPLGLAG |
| 205 | AWGRSAPLGLAG |
| 206 | AWGKSPLGLAG |
| 207 | GAFKSPLGLAG |
| 208 | SGRSAPLALAG |
| 209 | SGRSPLGLAG |
| 328 | RQRRSAAPLGLAG |
| 309 | RQRRSAPLGLAG |
| 310 | RQRRSPLGLAG |
| 325 | RQRRS-Nle-PLGLAG |
| 214 | RSGRSAPLGLAG |
| 215 | RSGKSAPLGLAG |
| 216 | FTARSAPLGLAG |
| 218 | FTAKSPLGLAG |
| 219 | RYGRSAPLGLAG |
| 220 | RYGRSPLGLAG |
| 327 | RYGKSAPLGLAG |
| 222 | KYGRSAPLGLAG |
| 223 | KWGRSAPLGLAG |
| 326 | KWGKSAPLGLAG |
| 225 | KWGRSPLGLAG |
| 311 | SGRVLTLRKAGPAGLVG |
| 312 | SGRVLTLRKAGPANLVG |
| 313 | SGRVLRKAGPAGLVG |
| 314 | SGRVLRKAGPANLVG |
| 305 | SGRVLGPAGLVG |
| 306 | SGRVLGPANLVG |
| 307 | SGRVLPAGLVG |
| 315 | SGRVLPANLVG |
| 316 | SGRVAGLVG |
| 317 | SGRVANLVG |
| 236 | SSRGRRGPLGLAG |
| 237 | SSRGPLGLAG |
| 238 | SSRGPRGLAG |
| 301 | SSRGPASNRRLPLGLAG |
| 318 | PASNRRLPLGLAG |
| 302 | SSRAVFRKNLGPLGLAG |
| 319 | SSRVFRKPANLAG |
| 304 | SGRVLTLRKAPWGLLE |
| 320 | SGRVLTLRKAALPLAM |
| 303 | SGRVLRKAGPQPLVD |
| 246 | SGRVLPLNLSG |
| 308 | SGRVLGPLNLSG |
| 321 | SSRGRRGPYMLQG |
| 322 | SSRGPYMLQG |
| 250 | SGRVLPLGLAG |
| 251 | SGRVLPMSLRM |
| 323 | SGRVLPLGMRA |
| 253 | SGRVLPLGLRA |
| 324 | SGRVLPYAMTA |
| 255 | SGRVLPLGFMG |
| 256 | GEEGEEPLGLAG |
| 257 | GPLGLAG |
| 258 | EAGRSANHTPAGLTGP |
| 259 | GEAGRSANHTPAGLTGP |

Further exemplary cleavable peptides according to the instant invention are shown below in Table 3A, which includes certain peptides of Table 2 above which have been prepared in a format for testing of cleavage efficiency. Additional exemplary peptides are shown in Table 3B. The peptides in Tables 3A and 3B have been prepared with a Forster resonance energy transfer (FRET) pair (N-terminal 2-amino benzoic acid (Abz) and C-terminal 3-nitro-tyrosine (Tyr(3-NO₂))). For sake of clarity, reference herein to the Linker number described in Table 3A or 3B refers to a construct which includes a peptide of the indicated sequence flanked by an N-terminal Abz group and a C-terminal 3-nitro-tyrosine group. For any reference to a SEQ ID NO listed in Table 3A or 3B, it is intended that the peptide referred to is the core sequence of the peptide listed in the table (i.e., to the sequence without considering the N-terminal Abz or the C-terminal Tyr(3-NO₂) residues). For example, a reference to LNK001 refers to the peptide of SEQ ID NO: 301 flanked by 1) an Abz group attached to the N-terminus of the first S residue of SEQ ID NO: 301 and 2) a Tyr(3-NO₂) group attached to the C-terminus of the last G residue of SEQ ID NO: 301. In contrast, any reference to SEQ ID NO: 301 refers to the sequence “SSRGPASNRRLPLGLAG.”

TABLE 3A
Selected experimental protease
cleavage sites.

| Linker | SEQ ID | |
|---|---|---|
| number | NO: | Sequence |
| LNK001 | 301 | SSRGPASNRRLPLGLAG |
| LNK002 | 302 | SSRAVFRKNLGPLGLAG |
| LNK003 | 303 | SGRVLRKAGPQPLVD |
| LNK004 | 304 | SGRVLTLRKAPWGLLE |
| LNK005 | 305 | SGRVLGPAGLVG |
| LNK006 | 306 | SGRVLGPANLVG |
| LNK007 | 307 | SGRVLPAGLVG |
| LNK008 | 308 | SGRVLGPLNLSG |
| LNK009 | 309 | RQRRSAPLGLAG |
| LNK010 | 310 | RQRRSPLGLAG |
| LNK011 | 311 | SGRVLTLRKAGPAGLVG |
| LNK012 | 312 | SGRVLTLRKAGPANLVG |
| LNK013 | 313 | SGRVLRKAGPAGLVG |
| LNK014 | 314 | SGRVLRKAGPANLVG |
| LNK015 | 315 | SGRVLPANLVG |
| LNK016 | 316 | SGRVAGLVG |
| LNK017 | 317 | SGRVANLVG |
| LNK018 | 318 | PASNRRLPLGLAG |
| LNK019 | 319 | SSRVFRKPANLAG |
| LNK020 | 320 | SGRVLTLRKAALPLAM |
| LNK021 | 321 | SSRGRRGPYMLQG |
| LNK022 | 322 | SSRGPYMLQG |
| LNK023 | 323 | SGRVLPLGMRA |
| LNK024 | 324 | SGRVLPYAMTA |
| LNK025 | 325 | RQRRS-Nle-PLGLAG |
| LNK026 | 326 | KWGKSAPLGLAG |
| LNK027 | 327 | RYGKSAPLGLAG |
| LNK028 | 328 | RQRRSAAPLGLAG |
| LNK029 | 329 | RQRRSVVGG |
| LNK030 | 330 | SPLGLAGS |
| LNK031 | 331 | RGRKVANLVG |
| LNK032 | 332 | RQRKVANLVG |
| LNK033 | 333 | RGRRVANLVG |
| LNK034 | 334 | RGRKSPANLVG |
| LNK035 | 335 | RGRKPYMLQG |
| LNK036 | 336 | RGRKPY-Nle-LQG |
| LNK037 | 337 | RGRKSPYMLQG |
| LNK038 | 338 | RGRKSPY-Nle-LQG |
| LNK039 | 339 | RGRKPQPLVD |
| LNK040 | 340 | RGRKSPQPLVD |
| LNK041 | 341 | RGRKSQPLVD |
| LNK042 | 342 | SGRVAPYMLQG |
| LNK043 | 343 | SGRVAPY-Nle-LQG |
| LNK044 | 344 | SGRVYMLQG |
| LNK045 | 345 | SGRVY-Nle-LQG |
| LNK046 | 346 | SGRVAPQPLVD |
| LNK047 | 347 | SGRVQPLVD |
| LNK048 | 348 | RGRRGP |

Further exemplary cleavable peptides according to the instant invention are shown below in Table 3B.

TABLE 3B
Additional Exemplary Peptides

| Linker | SEQ ID | |
|---|---|---|
| number | NO: | Sequence |
| LNK049 | 349 | RGRRPLGLAG |
| LNK050 | 350 | RGRRVANPLGLAGSG |
| LNK051 | 351 | RGRRPLGLAGGSG |
| LNK052 | 352 | RGRRHSSKLQ |
| LNK053 | 353 | SGRVANPLGGSG |
| LNK054 | 354 | SGRVANYFGKL |
| LNK055 | 355 | RGRRVANYFGKL |
| LNK056 | 356 | SGRPLGYFGKL |
| LNK057 | 357 | RGRRPLGYFGKL |
| LNK058 | 358 | RGRRVANPLGYFGKL |
| LNK059 | 359 | RGRRSGRAANLVRPLGYFGKL |
| LNK060 | 360 | RGRRAANLVRPLGYFGKL |
| LNK061 | 361 | HSSKLQYFGKL |
| LNK062 | 362 | RGRRHSSKLQPLGYFGKL |
| LNK063 | 363 | SGRHSSKLQPLGYFGKL |
| LNK064 | 364 | GSGSGSGS |
| LNK065 | 365 | SSLYSSPG |
| LNK066 | 366 | SSLQSSPG |
| LNK067 | 367 | SQYQSSPG |
| LNK068 | 368 | SQLYSSPG |
| LNK069 | 369 | SSQYSSPG |
| LNK070 | 370 | ISQYSSAT |
| LNK071 | 371 | KLYSSKQ |
| LNK072 | 372 | KLFSSKQ |
| LNK073 | 373 | RRLHYSL |
| LNK074 | 374 | RRLNYSL |
| LNK075 | 375 | RSSYRSL |
| LNK076 | 376 | RSSYYSL |
| LNK077 | 377 | KSKQHSL |
| LNK078 | 378 | HSSKLQL |
| LNK079 | 379 | GSSYYSGA |
| LNK080 | 380 | GSSVYSGR |
| LNK081 | 381 | SS-Nle-YSSAG |

In some embodiments, the cleavable peptide comprises an amino acid sequence having at least 80, 85, 90, 95% identity, or identical (100% identity) with peptide sequences in Table 1, 2, or 3A. In some embodiments, the cleavable peptide comprises an amino acid sequence having at least 80, 85, 90, 9500 identity, or identical (100% identity) with peptide sequences in Table 1, 2, 3A, or 3B. In some embodiments, the cleavable peptide comprises an amino acid sequence having at least 80, 85, 90, 950% identity, or identical (1000% identity) with peptide sequences in Table 2 or 3A. In some embodiments, the cleavable peptide comprises an amino acid sequence having at least 80, 85, 90, 9500 identity, or identical (1000% identity) with peptide sequences in Table 2, 3A, or 3B. In some embodiments, the cleavable peptide comprises an amino acid sequence having at least 80, 85, 90, 95% identity, or identical (100% identity) with peptide sequences in Table 2. In some embodiments, the cleavable peptide comprises an amino acid sequence set forth in Table 2 or Table 3A. In some embodiments, the cleavable peptide comprises an amino acid sequence set forth in Table 2, Table 3A, or Table 3B. In some embodiments, the cleavable peptide comprises an amino acid sequence set forth in Table 2. In some embodiments, the cleavable peptide comprises an amino acid sequence set forth in Table 2. In some embodiments, the cleavable peptide comprises an amino acid sequence set forth in Table 3A. In some embodiments, the cleavable peptide comprises an amino acid sequence set forth in Table 3B.

In some embodiments, the cleavable peptide comprises an amino acid sequence having at least 80, 85, 90, 95% identity, or an identical sequence to any one of SEQ ID NOs: 301-381. In some embodiments, the cleavable peptide comprises an amino acid sequence according to any one of SEQ ID NOs: 301-381 with up to 3 amino acid substitutions. In some embodiments, the cleavable peptide comprises an amino acid sequence according to any one of SEQ ID NOs: 301-381 with up to 2 amino acid substitutions. In some embodiments, the cleavable peptide comprises an amino acid sequence according to any one of SEQ ID NOs: 301-381 with up to 1 amino acid substitutions. In some embodiments, the cleavable peptide comprises an amino acid sequence according to any one of SEQ ID NOs: 301-381 with up to 3 conservative amino acid substitutions. In some embodiments, the cleavable peptide comprises an amino acid sequence according to any one of SEQ ID NOs: 301-381 with up to 2 conservative amino acid substitutions. In some embodiments, the cleavable peptide comprises an amino acid sequence according to any one of SEQ ID NOs: 301-381 with up to 1 conservative amino acid substitutions. In some embodiments, the cleavable peptide comprises an amino acid sequence identical to any one of SEQ ID NOs: 301-381.

In some embodiments, the cleavable peptide comprises an amino acid sequence having at least 80, 85, 90, 95% identity, or an identical sequence to any one of SEQ ID NOs: 301-342, 345-352, 354-363, 365-369, or 371-381. In some embodiments, the cleavable peptide comprises an amino acid sequence according to any one of SEQ ID NOs: 301-342, 345-352, 354-363, 365-369, or 371-381 with up to 3 amino acid substitutions. In some embodiments, the cleavable peptide comprises an amino acid sequence according to any one of SEQ ID NOs: 301-342, 345-352, 354-363, 365-369, or 371-381 with up to 2 amino acid substitutions. In some embodiments, the cleavable peptide comprises an amino acid sequence according to any one of SEQ ID NOs: 301-342, 345-352, 354-363, 365-369, or 371-381 with up to 1 amino acid substitutions. In some embodiments, the cleavable peptide comprises an amino acid sequence according to any one of SEQ ID NOs: 301-342, 345-352, 354-363, 365-369, or 371-381 with up to 3 conservative amino acid substitutions. In some embodiments, the cleavable peptide comprises an amino acid sequence according to any one of SEQ ID NOs: 301-342, 345-352, 354-363, 365-369, or 371-381 with up to 2 conservative amino acid substitutions. In some embodiments, the cleavable peptide comprises an amino acid sequence according to any one of SEQ ID NOs: 301-342, 345-352, 354-363, 365-369, or 371-381 with up to 1 conservative amino acid substitutions. In some embodiments, the cleavable peptide comprises an amino acid sequence identical to any one of SEQ ID NOs: 301-342, 345-352, 354-363, 365-369, or 371-381.

In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by an MMP. In some embodiments, the MMP is MMP-2, MMP7, and/or MMP-9. In some embodiments, the cleavable peptide is cleavable by a matriptase (e.g., human matriptase). In some embodiments, the cleavable peptide is cleavable by a plasminogen activator. In some embodiments, the cleavable peptide is cleavable by a legumain. In some embodiments, the cleavable peptide is cleavable by a kallikrein (e.g., Kallikrein-3) In some embodiments, the cleavable peptide is cleavable by a protease set forth in Table 1.

In some embodiments, the cleavable peptide is cleavable by multiple proteases. In some embodiments, the cleavable peptide is cleavable by multiple classes of proteases. In some embodiments, the cleavable peptide is cleavable by 2, 3, or 4 different proteases. In some embodiments, the cleavable peptide comprises multiple cleavage sites. In some embodiments, the cleavable peptide comprises 2, 3, 4, or more cleavage sites. In some embodiments, the cleavable peptide comprises 2 cleavage sites. In some embodiments, the cleavable peptide comprises 3 cleavage sites. In some embodiments, the cleavable peptide comprises 4 cleavage sites. In some embodiments, each of the cleavage sites is cleavable by a different protease. In some embodiments, a cleavage site can be cleaved by two or more different proteases (e.g., the site is shared by two or more different proteases).

In some embodiments, the cleavable peptide is cleavable by a matrix metalloprotease and a legumain. In some embodiments, the cleavable peptide is cleavable by a matrix metalloprotease and a matriptase. In some embodiments, the cleavable peptide is cleavable by a matrix metalloprotease and a plasminogen activator. In some embodiments, the cleavable peptide is cleavable by a legumain and a matriptase. In some embodiments, the cleavable peptide is cleavable by a legumain and a plasminogen activator. In some embodiments, the cleavable peptide is cleavable by a matriptase and a plasminogen activator. In some embodiments, the cleavable peptide is cleavable by a matrix metalloprotease, a legumain, and a matriptase. In some embodiments, the cleavable peptide is cleavable by a matrix metalloprotease, a matriptase, and a plasminogen activator. In some embodiments, the cleavable peptide is cleavable by a matrix metalloprotease, a legumain, and a plasminogen activator. In some embodiments, the cleavable peptide is cleavable by a matrix metalloprotease, a legumain, a plasminogen activator, and a matriptase.

In some embodiments, the cleavable peptide comprises one or more subsequences which aid in recognition by a protease. In some embodiments, the cleavable peptide comprises one or more of the subsequences SSR, SGG, SGR, RQR, RGR GKS, RKA, KAG, PAS, PAG, LAG, LVG, LVD, LQG, LLE, PQP, PWG, PLN, LSG, LAM, MRA, MTA, LPG, LTP, LTG, TGP, FTA, MKG, LPG, GEE, or VAN. In some embodiments, the cleavable peptide comprises 1, 2, 3, 4, 5, 6, or more of the subsequences SSR, SGG, SGR, RQR, RGR GKS, RKA, KAG, PAS, PAG, LAG, LVG, LVD, LQG, LLE, PQP, PWG, PLN, LSG, LAM, MRA, MTA, LPG, LTP, LTG, TGP, FTA, MKG, LPG, GEE, or VAN. In some embodiments, the cleavable peptide comprises one or more of the sequences PLG, PAN, PLN, PWG, PQP, or PAG. In some embodiments, the cleavable peptide comprises 1, 2, 3, 4, 5, or 6 of the sequences PLG, PAN, PLN, PWG, PQP, or PAG. In some embodiments, the cleavable peptide comprises a sequence LAG, LVG, or LQG.

In some embodiments, the cleavable peptide comprises one or more of the subsequences SSRV (SEQ ID NO: 401), SSRG (SEQ ID NO: 402), SGRV (SEQ ID NO: 403), RKAG (SEQ ID NO: 404), RQRR (SEQ ID NO: 405), RGRR (SEQ ID NO: 406), RGRK (SEQ ID NO: 407), RQRK (SEQ ID NO: 408), GKSA (SEQ ID NO: 409), MLQG (SEQ ID NO: 410), Nle-LQG, PASN (SEQ ID NO: 412), or PLVD (SEQ ID NO: 413). In some embodiments, the cleavable peptide comprises 1, 2, 3, 4, or more of the subsequences SSRV (SEQ ID NO: 401), SSRG (SEQ ID NO: 402), SGRV (SEQ ID NO: 403), RKAG (SEQ ID NO: 404), RQRR (SEQ ID NO: 405), RGRR (SEQ ID NO: 406), RGRK (SEQ ID NO: 407), RQRK (SEQ ID NO: 408), GKSA (SEQ ID NO: 409), MLQG (SEQ ID NO: 410), Nle-LQG, PASN (SEQ ID NO: 412), or PLVD (SEQ ID NO: 413). In some embodiments, the cleavable peptide comprises a sequence SSRG (SEQ ID NO: 402), SSRA (SEQ ID NO: 414), SGRV (SEQ ID NO: 403), or SSRV (SEQ ID NO: 401). In some embodiments, the cleavable peptide comprises a sequence RQRR (SEQ ID NO: 405), RGRK (SEQ ID NO: 407), or RGRR (SEQ ID NO: 406).

In some embodiments, the cleavable peptide comprises one or more of the subsequences SGRVL (SEQ ID NO: 423), RQRRS (SEQ ID NO: 415), RGRKS (SEQ ID NO: 416), RGRKP (SEQ ID NO: 417), SGRVA (SEQ ID NO: 418), SGRVY (SEQ ID NO: 419), SGGPG (SEQ ID NO: 420), AGLVG (SEQ ID NO: 421), or ANLVG (SEQ ID NO: 422). In some embodiments, the cleavable peptide comprises 1, 2, 3, or more of the subsequences SGRVL (SEQ ID NO: 423), RQRRS (SEQ ID NO: 415), RGRKS (SEQ ID NO: 416), RGRKP (SEQ ID NO: 417), SGRVA (SEQ ID NO: 418), SGRVY (SEQ ID NO: 419), SGGPG (SEQ ID NO: 420), AGLVG (SEQ ID NO: 421), or ANLVG (SEQ ID NO: 422). In some embodiments, the cleavable peptide comprises a sequence RQRRS (SEQ ID NO: 415) or RQRRV (SEQ ID NO: 424).

In some embodiments, the cleavable peptide comprises one or more of the subsequences PLGLAG (SEQ ID NO: 129), PQPLVD (SEQ ID NO: 425), PWGLLE (SEQ ID NO: 426), PAGLVG (SEQ ID NO: 427), PANLVG (SEQ ID NO: 428), PLNLSG (SEQ ID NO: 429), AGLVG (SEQ ID NO: 430), ANLVG (SEQ ID NO: 431), PANLAG (SEQ ID NO: 432), GEEGEE (SEQ ID NO: 433), MKGLPG (SEQ ID NO: 434), RGRKSP (SEQ ID NO: 435), RGRKPY (SEQ ID NO: 436), RGRKPQ (SEQ ID NO: 437), PAGLTP (SEQ ID NO: 438), or PAGLTG (SEQ ID NO: 439). In some embodiments, the cleavable peptide comprises 1, 2, 3, or more of the subsequences PLGLAG (SEQ ID NO: 129), PQPLVD (SEQ ID NO: 425), PWGLLE (SEQ ID NO: 426), PAGLVG (SEQ ID NO: 427), PANLVG (SEQ ID NO: 428), PLNLSG (SEQ ID NO: 429), AGLVG (SEQ ID NO: 430), ANLVG (SEQ ID NO: 431), PANLAG (SEQ ID NO: 432), GEEGEE (SEQ ID NO: 433), MKGLPG (SEQ ID NO: 434), RGRKSP (SEQ ID NO: 435), RGRKPY (SEQ ID NO: 436), RGRKPQ (SEQ ID NO: 437), PAGLTP (SEQ ID NO: 438), or PAGLTG (SEQ ID NO: 439).

In some embodiments, the cleavable peptide comprises a subsequence EAGRSANHT (SEQ ID NO: 440).

In some embodiments, the cleavable peptide comprises one or more of the peptides: SSRGPASNRRLPLGLAG (SEQ ID NO: 301), SSRAVFRKNLGPLGLAG (SEQ ID NO: 302), SGRVLRKAGPQPLVD (SEQ ID NO: 303), SGRVLTLRKAPWGLLE (SEQ ID NO: 304), SGRVLGPAGLVG (SEQ ID NO: 305), SGRVLGPANLVG (SEQ ID NO: 306), SGRVLPAGLVG (SEQ ID NO: 307), SGRVLGPLNLSG (SEQ ID NO: 308), RQRRSAPLGLAG (SEQ ID NO: 309), RQRRSPLGLAG (SEQ ID NO: 310), SGRVLTLRKAGPAGLVG (SEQ ID NO: 311), SGRVLTLRKAGPANLVG (SEQ ID NO: 312), SGRVLRKAGPAGLVG (SEQ ID NO: 313), SGRVLRKAGPANLVG (SEQ ID NO: 314), SGRVLPANLVG (SEQ ID NO: 315), SGRVAGLVG (SEQ ID NO: 316), SGRVANLVG (SEQ ID NO: 317), PASNRRLPLGLAG (SEQ ID NO: 318), SSRVFRKPANLAG (SEQ ID NO: 319), SGRVLTLRKAALPLAM (SEQ ID NO: 320), SSRGRRGPYMLQG (SEQ ID NO: 321), SSRGPYMLQG (SEQ ID NO: 322), SGRVLPLGMRA (SEQ ID NO: 323), SGRVLPYAMTA (SEQ ID NO: 324), RQRRS-Nle-PLGLAG (SEQ ID NO: 325), KWGKSAPLGLAG (SEQ ID NO: 326), RYGKSAPLGLAG (SEQ ID NO: 327), RQRRSAAPLGLAG (SEQ ID NO: 328), RQRRSVVGG (SEQ ID NO: 329), SPLGLAGS (SEQ ID NO: 330), RGRKVANLVG (SEQ ID NO: 331), RQRKVANLVG (SEQ ID NO: 332), RGRRVANLVG (SEQ ID NO: 333), RGRKSPANLVG (SEQ ID NO: 334), RGRKPYMLQG (SEQ ID NO: 335), RGRKPY-Nle-LQG (SEQ ID NO: 336), RGRKSPYMLQG (SEQ ID NO: 337), RGRKSPY-Nle-LQG (SEQ ID NO: 338), RGRKPQPLVD (SEQ ID NO: 339), RGRKSPQPLVD (SEQ ID NO: 340), RGRKSQPLVD (SEQ ID NO: 341), SGRVAPYMLQG (SEQ ID NO: 342), SGRVAPY-Nle-LQG (SEQ ID NO: 343), SGRVYMLQG (SEQ ID NO: 344), SGRVY-Nle-LQG (SEQ ID NO: 345), SGRVAPQPLVD (SEQ ID NO: 346), SGRVQPLVD (SEQ ID NO: 347), RGRRGP (SEQ ID NO: 348), FTARSAPLGLAG (SEQ ID NO: 216), FTAKSPLGLAG (SEQ ID NO: 218), SSRGPLGLAG (SEQ ID NO: 237), SSRGPRGLAG (SEQ ID NO: 238), SGGPGPAGMKGLPGS (SEQ ID NO: 101), MKGLPGS (SEQ ID NO: 441), GEEGEEPLGLAG (SEQ ID NO: 256), PLGLAG (SEQ ID NO: 129), LAG, KPLGLAG (SEQ ID NO: 442), KKPLGLAG (SEQ ID NO: 443), GPLGLAG (SEQ ID NO: 257), GEAGRSANHTPAGLTP (SEQ ID NO: 444), or EAGRSANHTPAGLTGP (SEQ ID NO: 258). In some embodiments, the cleavable peptide comprises one or more of the peptides: SSRGPASNRRLPLGLAG (SEQ ID NO: 301), SSRAVFRKNLGPLGLAG (SEQ ID NO: 302), SGRVLRKAGPQPLVD (SEQ ID NO: 303), SGRVLTLRKAPWGLLE (SEQ ID NO: 304), SGRVLGPAGLVG (SEQ ID NO: 305), SGRVLGPANLVG (SEQ ID NO: 306), SGRVLPAGLVG (SEQ ID NO: 307), SGRVLGPLNLSG (SEQ ID NO: 308), RQRRSAPLGLAG (SEQ ID NO: 309), RQRRSPLGLAG (SEQ ID NO: 310), SGRVLTLRKAGPAGLVG (SEQ ID NO: 311), SGRVLTLRKAGPANLVG (SEQ ID NO: 312), SGRVLRKAGPAGLVG (SEQ ID NO: 313), SGRVLRKAGPANLVG (SEQ ID NO: 314), SGRVLPANLVG (SEQ ID NO: 315), SGRVAGLVG (SEQ ID NO: 316), SGRVANLVG (SEQ ID NO: 317), PASNRRLPLGLAG (SEQ ID NO: 318), SSRVFRKPANLAG (SEQ ID NO: 319), SGRVLTLRKAALPLAM (SEQ ID NO: 320), SSRGRRGPYMLQG (SEQ ID NO: 321), SSRGPYMLQG (SEQ ID NO: 322), SGRVLPLGMRA (SEQ ID NO: 323), SGRVLPYAMTA (SEQ ID NO: 324), RQRRS-Nle-PLGLAG (SEQ ID NO: 325), KWGKSAPLGLAG (SEQ ID NO: 326), RYGKSAPLGLAG (SEQ ID NO: 327), or RQRRSAAPLGLAG (SEQ ID NO: 328). In some embodiments, the cleavable peptide comprises one or more of the peptides: RGRRPLGLAG (SEQ ID NO: 349), RGRRVANPLGLAGSG (SEQ ID NO: 350), RGRRPLGLAGGSG (SEQ ID NO: 351), RGRRHSSKLQ (SEQ ID NO: 352), SGRVANPLGGSG (SEQ ID NO: 353), SGRVANYFGKL (SEQ ID NO: 354), RGRRVANYFGKL (SEQ ID NO: 355), SGRPLGYFGKL (SEQ ID NO: 356), RGRRPLGYFGKL (SEQ ID NO: 357), RGRRVANPLGYFGKL (SEQ ID NO: 358), RGRRSGRAANLVRPLGYFGKL (SEQ ID NO: 359), RGRRAANLVRPLGYFGKL (SEQ ID NO: 360), HSSKLQYFGKL (SEQ ID NO: 361), RGRRHSSKLQPLGYFGKL (SEQ ID NO: 362), SGRHSSKLQPLGYFGKL (SEQ ID NO: 363), GSGSGSGS (SEQ ID NO: 364), SSLYSSPG (SEQ ID NO: 365), SSLQSSPG (SEQ ID NO: 366), SQYQSSPG (SEQ ID NO: 367), SQLYSSPG (SEQ ID NO: 368), SSQYSSPG (SEQ ID NO: 369), ISQYSSAT (SEQ ID NO: 370), KLYSSKQ (SEQ ID NO: 371), KLFSSKQ (SEQ ID NO: 372), RRLHYSL (SEQ ID NO: 373), RRLNYSL (SEQ ID NO: 374), RSSYRSL (SEQ ID NO: 375), RSSYYSL(SEQ ID NO: 376), KSKQHSL (SEQ ID NO: 377), HSSKLQL (SEQ ID NO: 378), GSSYYSGA (SEQ ID NO: 379), GSSVYSGR (SEQ ID NO: 380), or SS-Nle-YSSAG (SEQ ID NO: 381).

In some embodiments, the cleavable peptide is attached to at least one additional group A¹. In some embodiments, the cleavable peptide is attached to a plurality of additional groups A¹. In some embodiments, the cleavable peptide is attached to at least two additional groups A¹. In embodiments wherein the cleavable peptide is attached to a plurality of additional groups A¹, each A¹ can be the same or different.

In some embodiments, the cleavable peptide is attached to at least two different additional groups A¹. In some embodiments, each of the two different additional groups A¹ is independently a polypeptide (e.g. the cleavable peptide comprises a first additional group A¹ polypeptide attached to the N-terminal amine of the cleavable peptide (such as by a peptide bond) and a second additional group A¹ polypeptide attached to the C-terminal carboxyl of the cleavable peptide (such as by a peptide bond)).

In some embodiments wherein the cleavable peptide is attached to at least two additional groups A¹, the two additional groups A¹ are positioned on the cleavable peptide such that cleavage of a cleavable site of the cleavable peptide causes the two additional groups A¹ to no longer be covalently linked. In some embodiments, cleavage of any one of the plurality of cleavage sites on the cleavable peptide causes the two additional groups A¹ to no longer be covalently linked.

In some embodiments, the cleavable peptide is attached to an additional group A¹ at two locations of the cleavable peptide to form a cyclic structure. In some embodiments, cleavage of a cleavage site of the cleavable peptide breaks the cyclic structure. In some embodiments, cleavage of any one of the plurality of protease cleavage sites breaks the cyclic structure.

In some embodiments, each of the additional groups A¹ is independently selected from a polypeptide, a nucleic acid, a polysaccharide, a lipid, an antibody, an organic biopolymer, a chemical polymer, a drug, a nanoparticle, a dye, or a bio-organic molecule. In some embodiments, each additional group A¹ is independently a polypeptide (e.g., the cleavable peptide is attached to two different polypeptide, such as in a fusion protein wherein the cleavable peptide is flanked by different domains of the fusion protein).

In some embodiments, each of the additional groups A¹ is optionally attached to the cleavable peptide by a linker. The linker can be any suitable group capable of forming a chain of atoms connected by bonds (e.g., covalent bonds) between the cleavable peptide and the additional group A¹. Non-limiting examples of linkers include polymers (e.g., chemical polymers, such as poly(ethylene glycol)), hydrocarbon groups (optionally interspersed with heteroatoms or substituted with heteroatoms), peptide linkers (e.g., glycine and serine rich peptide sequences), and the like. In some embodiments, the cleavable peptide is conjugated to the additional group A¹ (e.g., by a conjugation reaction). In some embodiments, the cleavable peptide is attached to the additional group A¹ (or a plurality of additional groups A¹) as a fusion gene product.

In some embodiments, the cleavable peptide is incorporated into the amino acid sequence of a protein (e.g., forms one part or subunit of a larger protein sequence, such as an internal sequence or between domains of the protein, or between different fused genes in a fusion protein). In some embodiments, the cleavable peptide is comprised in an internal portion of the protein. In some embodiments, the protein is a recombinant protein. In some embodiments, the protein is a fusion protein. In some embodiments, the cleavable peptide is positioned between domains of the fusion protein. In some embodiments, the protein is an activatable protein (e.g., cleavage of the cleavable peptide alters the activity of the protein (or the cleaved subunits as compared to the intact protein before cleavage)). In some embodiments, the cleavable peptide is appended to the N- or C-terminus of the protein.

In some embodiments, the cleavable peptide is attached to a side chain of an amino acid of a protein. In some embodiments, the protein is an activatable protein. In some embodiments, the cleavable peptide attached to a side chain of an amino acid of a protein is further connected to an additional group (e.g., an additional group A¹). In some embodiments, the cleavable peptide attached to a side chain of an amino acid of a protein is attached to the protein at another point of attachment (e.g., another side chain of another amino acid, or the N- or C-terminus of the protein) to form a cyclic structure.

### Activatable Protein Compositions

In one aspect, provided herein is an activatable protein composition. In one embodiment, the activatable protein comprises a first protein molecule and a first cleavable peptide, wherein the cleavable peptide is attached to the first protein at a first site via an amino acid side chain, wherein the activatable protein displays altered activity after cleavage of the first cleavable peptide as compared to the activity of the activatable protein prior to cleavage of the first cleavable peptide.

In one embodiment, the conditionally activated protein can be used as treatment of diseases such as cancer, immune diseases, or infectious diseases. In some embodiments, the conditionally activated protein comprises cleavable peptides that inhibits the activity of the protein in healthy tissue environments. In some embodiments, the cleavable peptide blocks the ability of the proteins to engage in binding to target molecules (e.g. receptors). In some embodiments, the cleavable peptides inhibit the enzymatic activity of the proteins. In some embodiments, the cleavable peptides attach to one or more residues of the proteins. In some embodiments, the cleavable peptide attaches to at least a side chain of an internal residue and may optionally attach to a second residue selected from an amino terminal residue, a carboxy terminal residue, and a side chain of an internal residue. In some embodiments, the cleavable peptide acts as an environmental sensor. In some embodiments, environmental cues (e.g. proteases) cause the cleavage of the cleavable peptide. In some embodiments, cleavage of the cleavable peptides comprises cleavage at one or more cleavage sites. In some embodiments, cleavage of the cleavable peptide at one or more cleavage sites enhances the activity of the protein relative to the protein comprising the cleavable peptide. In some embodiments, the attachment of the cleavable peptide to internal residue side chains or to multiple points in the protein provides more complete inhibition of an activatable protein wherein cleavage of the cleavable peptide causes a greater increase in activity of the proteins than are possible in other methods.

In some embodiments, the activatable protein comprises:

P¹-L¹-(G¹)_{q},

wherein, P¹ is a first protein having a first site, which is an amino acid side chain; each G¹ independently comprises a protease-cleavable peptide, wherein q is an integer between 1 to 50; L¹ is a covalent bond between G¹ and P¹ or a divalent linker bound to P¹ at the first site and to G¹; wherein G¹ binds to P¹ via the first site. In some embodiments, the cleavable peptide attached to the activatable protein is a protease cleavable peptide.

In some embodiments, the amino acid in the first protein to which the cleavable peptide is attached interacts with a ligand.

In some embodiments, the activatable protein comprises a cyclic protein of formula:

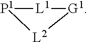

In some embodiments, L² is a branched or unbranched polyethylene glycol linker. In some embodiments, the activatable protein comprises more than one L¹ and/or L².

In some embodiments, the activatable protein comprises at least one protein linked to a cleavable peptide. In some embodiments, the activatable protein comprises more than one protein molecules and/or cleavable peptides, wherein the cleavable peptides are attached to the proteins via amino acid side chains. In some embodiments, the one or more protein molecules are recombinant and/or synthetic. In some embodiments, the first protein molecule is a recombinant or synthetic cytokine, or a derivative thereof. In some embodiments, the first protein molecule is a recombinant or synthetic interleukin, or a derivative thereof. In some embodiments, the first protein molecule is a synthetic interleukin.

In some embodiments, the first protein molecule is a synthetic (i.e., prepared from one or more chemically synthesized peptides) IL-2. In some embodiments, cleavage of a first cleavable peptide enhances the binding of the IL-2 protein to one or more IL-2 receptor subunits.

In some embodiments, the activatable protein is from at least about 50 amino acids in length to about 500 amino acids in length. In some embodiments, the activatable protein is from at least about 50 amino acids in length to about 75 amino acids in length, about 50 amino acids in length to about 100 amino acids in length, about 50 amino acids in length to about 125 amino acids in length, about 50 amino acids in length to about 150 amino acids in length, about 50 amino acids in length to about 200 amino acids in length, about 50 amino acids in length to about 250 amino acids in length, about 50 amino acids in length to about 300 amino acids in length, about 50 amino acids in length to about 500 amino acids in length, about 75 amino acids in length to about 100 amino acids in length, about 75 amino acids in length to about 125 amino acids in length, about 75 amino acids in length to about 150 amino acids in length, about 75 amino acids in length to about 200 amino acids in length, about 75 amino acids in length to about 250 amino acids in length, about 75 amino acids in length to about 300 amino acids in length, about 75 amino acids in length to about 500 amino acids in length, about 100 amino acids in length to about 125 amino acids in length, about 100 amino acids in length to about 150 amino acids in length, about 100 amino acids in length to about 200 amino acids in length, about 100 amino acids in length to about 250 amino acids in length, about 100 amino acids in length to about 300 amino acids in length, about 100 amino acids in length to about 500 amino acids in length, about 125 amino acids in length to about 150 amino acids in length, about 125 amino acids in length to about 200 amino acids in length, about 125 amino acids in length to about 250 amino acids in length, about 125 amino acids in length to about 300 amino acids in length, about 125 amino acids in length to about 500 amino acids in length, about 150 amino acids in length to about 200 amino acids in length, about 150 amino acids in length to about 250 amino acids in length, about 150 amino acids in length to about 300 amino acids in length, about 150 amino acids in length to about 500 amino acids in length, about 200 amino acids in length to about 250 amino acids in length, about 200 amino acids in length to about 300 amino acids in length, about 200 amino acids in length to about 500 amino acids in length, about 250 amino acids in length to about 300 amino acids in length, about 250 amino acids in length to about 500 amino acids in length, or about 300 amino acids in length to about 500 amino acids in length. In some embodiments, the activatable protein is from at least about 50 amino acids in length, about 75 amino acids in length, about 100 amino acids in length, about 125 amino acids in length, about 150 amino acids in length, about 200 amino acids in length, about 250 amino acids in length, about 300 amino acids in length, or about 500 amino acids in length. In some embodiments, the activatable protein is from at least at least about 50 amino acids in length, about 75 amino acids in length, about 100 amino acids in length, about 125 amino acids in length, about 150 amino acids in length, about 200 amino acids in length, about 250 amino acids in length, or about 300 amino acids in length. In some embodiments, the activatable protein is from at least at most about 75 amino acids in length, about 100 amino acids in length, about 125 amino acids in length, about 150 amino acids in length, about 200 amino acids in length, about 250 amino acids in length, about 300 amino acids in length, or about 500 amino acids in length.

### Protease Cleavable Peptides in Activatable Proteins

In some embodiments, the activatable protein comprises at least one cleavable peptide attached to the first protein molecule via an amino acid side chain. The cleavable peptide can be any of the cleavable peptides described herein supra. In some embodiments, the activatable protein comprises multiple cleavable peptides. In some embodiments, the cleavable peptide comprises a protease-cleavable peptide, wherein each G¹ is independently of the formula:

*—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—**,
wherein:
X¹ is S, R, P, K, F, G, or absent;
X² is S, G, Q, A, W, Y, T, E or absent;
X³ is R, S, G, A, E, or absent;
X⁴ is G, A, V, R, N, K, P, or absent;
X⁵ is P, V, L, S, F, R, A, Y, Q, G, E or absent;
X⁶ is A, F, R, T, G, P, Nle, V, Y, Q, E, S, or absent;
X⁷ is S, R, K, L, Y, A, G, P, Q, or absent;
X⁸ is N, K, A, R, P, G, or absent;
X⁹ is N, G, K, L, H, or absent;
X¹⁰ is R, L, A, K, T, or absent;
X¹¹ is R, G, A, K, S or absent;
X¹² is P, M, or absent;
X¹³ is L, Q, W, A, Y, G, R, K, or absent;
X¹⁴ is G, P, N, M, Nle, or absent;
X¹⁵ is L, R, T, or absent;
X¹⁶ is A, L, V, S, Q, P, T, or absent;
X¹⁷ is G, E, M, or D; and
X¹⁸ is S, P, or absent;
X¹⁹ is absent, —NH-T¹ or —(C═O)-T¹, wherein T¹ is a terminal group, a group B¹,
   wherein B¹ is a blocking moiety, or a linker L², wherein L² is covalently bonded to the protein P¹ at a second site separate from the first site to form a cyclic protein with P¹;
   * represents either the C- or N-terminus of the cleavable peptide and ** represents the other of the C- or N-terminus of the cleavable peptide; wherein one or more side chains of any of amino acids X¹—X¹⁹ can be directly or indirectly covalently bonded to one or more groups B², wherein B² is a blocking moiety.

In some embodiments, the terminal group in the cleavable peptide can be a blocking moiety (for example, B¹/B²), wherein the blocking moiety can be any molecule that blocks a portion of the first protein molecule such that the first protein molecule is at least partially inactive. In some embodiments, the blocking moiety can be any molecule that blocks a portion of any protein molecule in the activatable protein. In some embodiments, the cleavable peptide comprises multiple blocking moieties. In some embodiments, the blocking moieties are randomly present at any position within the cleavable peptide sequence. In some embodiments, the activatable protein comprises multiple blocking moieties, B¹ to B⁵⁰. In some embodiments, the blocking moieties are randomly present at any position within the activatable protein.

In some embodiments, the blocking moiety (B¹/B²) is a protein molecule or another biological macromolecule including but not limited to nucleic acids, fatty acids, polysaccharides, antibodies, organic biopolymers, organic polymers, or an organic compound etc.

In some embodiments, the blocking moiety is a linker L², wherein L² is covalently bonded to the first protein molecule at a second site on the amino acid side chain, separate from the first site to form a cyclic activatable protein. In some embodiments, L² comprises a straight or branched PEGₚ, wherein p is an integer from 1 to 50 and represents the number of ethylene glycol monomer subunits in PEGₚ.

In some embodiments, the cleavage of the cleavable peptide leaves about 1 amino acid to about 10 amino acids attached to the activatable protein. In some embodiments, the cleavage of the cleavable peptide leaves about 1 amino acid to about 2 amino acids, about 1 amino acid to about 3 amino acids, about 1 amino acid to about 4 amino acids, about 1 amino acid to about 5 amino acids, about 1 amino acid to about 6 amino acids, about 1 amino acid to about 7 amino acids, about 1 amino acid to about 8 amino acids, about 1 amino acid to about 9 amino acids, about 1 amino acid to about 10 amino acids, about 2 amino acids to about 3 amino acids, about 2 amino acids to about 4 amino acids, about 2 amino acids to about 5 amino acids, about 2 amino acids to about 6 amino acids, about 2 amino acids to about 7 amino acids, about 2 amino acids to about 8 amino acids, about 2 amino acids to about 9 amino acids, about 2 amino acids to about 10 amino acids, about 3 amino acids to about 4 amino acids, about 3 amino acids to about 5 amino acids, about 3 amino acids to about 6 amino acids, about 3 amino acids to about 7 amino acids, about 3 amino acids to about 8 amino acids, about 3 amino acids to about 9 amino acids, about 3 amino acids to about 10 amino acids, about 4 amino acids to about 5 amino acids, about 4 amino acids to about 6 amino acids, about 4 amino acids to about 7 amino acids, about 4 amino acids to about 8 amino acids, about 4 amino acids to about 9 amino acids, about 4 amino acids to about 10 amino acids, about 5 amino acids to about 6 amino acids, about 5 amino acids to about 7 amino acids, about 5 amino acids to about 8 amino acids, about 5 amino acids to about 9 amino acids, about 5 amino acids to about 10 amino acids, about 6 amino acids to about 7 amino acids, about 6 amino acids to about 8 amino acids, about 6 amino acids to about 9 amino acids, about 6 amino acids to about 10 amino acids, about 7 amino acids to about 8 amino acids, about 7 amino acids to about 9 amino acids, about 7 amino acids to about 10 amino acids, about 8 amino acids to about 9 amino acids, about 8 amino acids to about 10 amino acids, or about 9 amino acids to about 10 amino acids.

In one embodiment, the cleavage of the cleavable peptide leaves about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids. In some embodiments, the cleavage of the cleavable peptide leaves at least about 1 amino acid, about 2 amino acids, about 3 amino acids, about 4 amino acids, about 5 amino acids, about 6 amino acids, about 7 amino acids, about 8 amino acids, or about 9 amino acids. In some embodiments, the cleavage of the cleavable peptide leaves at most about 2 amino acids, about 3 amino acids, about 4 amino acids, about 5 amino acids, about 6 amino acids, about 7 amino acids, about 8 amino acids, about 9 amino acids, or about 10 amino acids.

In some embodiments, the cleavable peptide is attached to the first site on the first protein via a covalent bond, wherein the first site comprises a side chain of an amino acid residue in the first protein. In some embodiments, the cleavable peptide is attached to side chain of an amino acid residue in the first protein via multiple covalent bonds. In some embodiments, the cleavable peptide comprises at least one protease cleavage site. In some embodiments, site specific cleavage at the cleavable peptide modulates the activity of the activatable protein.

In some embodiments, the cleavable peptide attached to the activatable protein is cleavable by a protease selected from a kallikrein, thrombin, chymase, carboxyprotease A, and elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleaved by multiple proteases.

In some embodiments, the cleavable peptide comprises an amino acid sequence having at least 80, 85, 90, 95% identity, or identical (100% identity) with peptide sequences in Table 1, 2, or 3A. In some embodiments, the cleavable peptide comprises an amino acid sequence having at least 80, 85, 90, 95% identity, or identical (100% identity) with peptide sequences in Table 1, 2, 3A, or 3B.

### Sites of Attachment of the Cleavable Peptide

In some embodiments, the activatable protein comprises one or more cleavable peptides, wherein the cleavable peptides are covalently attached to the first protein via an amino acid residue selected from an amino terminal residue, a carboxy terminal residue, and a side chain of any internal residue.

In some embodiments, the one or more cleavable peptides are covalently attached to the one or more protein molecules via an amino acid side chain. In some embodiments, the C-terminus of the cleavable peptide is attached to the side chain of the amino acid of the first protein. In some embodiments, the N-terminus of the cleavable peptide is attached to the side chain of the amino acid of the first protein. In some embodiments, the amino acid residue of the first protein to which the cleavable peptide is attached is a lysine, glutamine, aspartate, arginine, tyrosine, serine, threonine, cysteine, or a non-naturally occurring amino acid, such as homoserine, homolysine, homoarginine, etc. In some embodiments, the amino acid residue of the first protein to which the cleavable peptide is attached is a lysine or glutamine. In some embodiments, the amino acid residue to which the cleavable peptide is attached is a lysine. In some embodiments, the amino acid residue to which the cleavable peptide is attached is a glutamate. In some embodiments, the amino acid residue to which the cleavable peptide is attached is a diaminobutyric acid. In some embodiments, the amino acid residue to which the cleavable peptide is attached is substituted relative to the amino acid at the corresponding position in the wild type version of the protein.

In some embodiments, the cleavable peptide is attached to the activatable protein at an additional point of attachment. In some embodiments, the additional point of attachment of the cleavable peptide is the N-terminus or C-terminus of the activatable protein. In some embodiments, the additional point of attachment is to the side chain of another amino acid residue in the activatable protein. In some embodiments, the cleavable peptide is attached to another moiety. In some embodiments, cleavage of the cleavable peptide causes the additional moiety to no longer be attached to the protein. In some embodiments, the cleavable peptide is attached to the side chain of the amino acid residue through a linking group.

### An Activatable IL-2 Peptide

In one embodiment, provided herein is an activatable IL-2 polypeptide comprising a cleavable peptide attached to a side chain of an amino acid of the IL-2 polypeptide, wherein the IL-2 polypeptide displays an enhanced ability to bind at least one IL-2 receptor subunit after cleavage of the cleavable peptide as compared to the activity of the activatable IL-2 polypeptide before cleavage.

In some embodiments, the activatable IL-2 polypeptide comprises a cleavable moiety attached to a residue in the region of residues 1-35 of the IL-2 polypeptide, wherein residue position numbering is based on SEQ ID NO: 1 as a reference sequence; and wherein the IL-2 polypeptide exhibits a greater affinity for the IL-2 receptor beta subunit after cleavage of the cleavable moiety compared to the activatable IL-2 polypeptide before cleavage of the cleavable moiety. In some embodiments, activatable IL-2 proteins are shown in Table 4. In some embodiments, a cleavable peptide of Table 2 or Table 3A is incorporated into an activatable IL-2 polypeptide of Table 4 in place of the cleavable peptide denoted in the constructs therein (i.e., the underlined peptide sequences), or into an analogous IL-2 polypeptide. In some embodiments, a cleavable peptide of Table 3B is incorporated into an activatable IL-2 polypeptide of Table 4 in place of the cleavable peptide denoted in the constructs therein (i.e., the underlined peptide sequences), or into an analogous IL-2 polypeptide.

TABLE 4
Activatable IL-2 Polypeptides

| | Substitute | |
|---|---|---|
| SEQ ID NO: | Ref | Sequence |
| 1 | | APTSSSSTKKTQLQLEHLLLDLQMILNGINNYKNPKLTRMLTFKFYMP |
| (WT-IL-2) | | KKATELKHLQCLEEELKPLEEVLNLAQSKNFHLRPRDLISNINVIVLE |
| | | LKGSETTFMCEYADETATIVEFLNRWITFCQSIISTLT |
| 2 | | APTSSSTKKTQLQLEHLLLDLQ(Nle)ILNGINNYKNPKLTR(Nle)L(Hse) |
| No | | YKFY(Nle)PKKATELKHLQCLEEELKPLEEVL(Hse)LAQSKNFHLRPR |
| PEGs | | DLISNINVIVLELKGSETTF(Hse)CEYADETATIVEFLNRWITFSQSIIST |
| | | LT |
| 3 | CMP-003 | APTSSSTKKTQLQLEHLLLDLQ-Nle-ILNGINNYKNPKLTR-Nle-L-Hse-Yn3-K |
| | | F-Ygp-NlePKKATELKHLQCLEEELKPLEEVL-Hse-LAQSKNFHLRPRDLSINI |
| | | NVIVLELKGSETTF-Hse-CEYADETATIVEFLNRWITFSQSIISLT |
| 4 | CMP-118 | 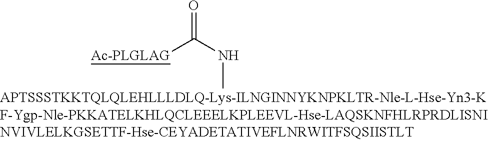 |
| 5 | CMP-119 | 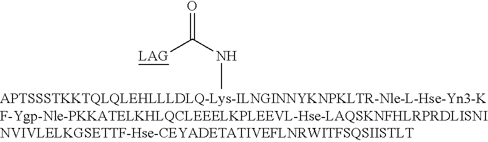 |
| 6 | CMP-120 | 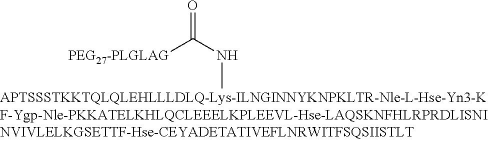 |
| 7 | CMP-121 | 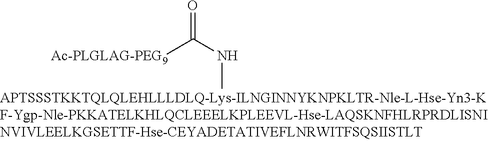 |
| 8 | CMP-122 | 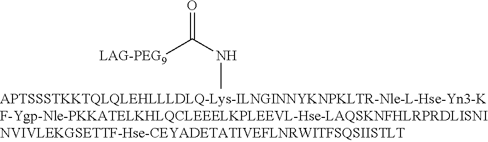 |
| 9 | CMP-123 | 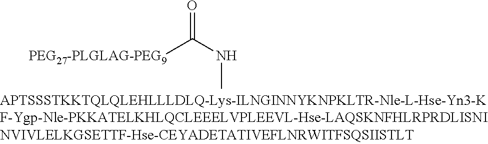 |
| 10 | CMP-124 | 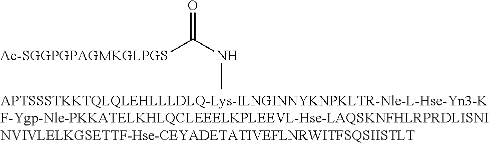 |
| 11 | CMP-125 | 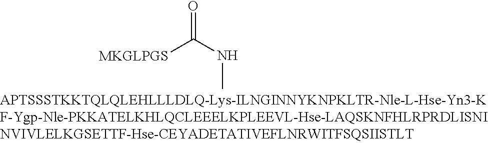 |
| 12 | CMP-126 | 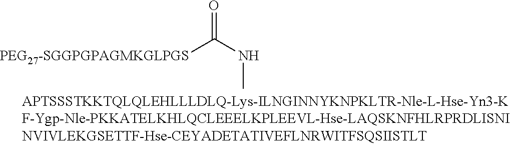 |
| 13 | CMP-127 | 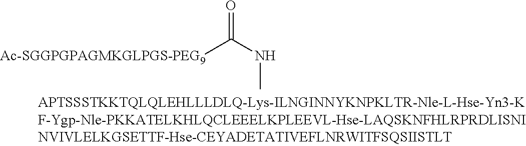 |
| 14 | CMP-128 | 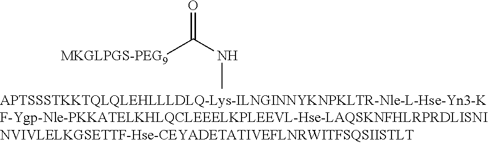 |
| 15 | CMP-129 | 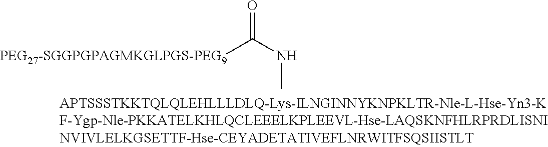 |
| 16 | CMP-141 | 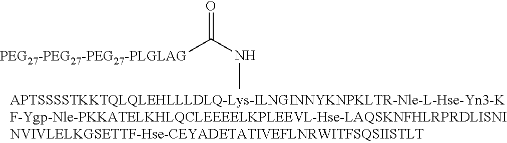 |
| 17 | CMP-142 | 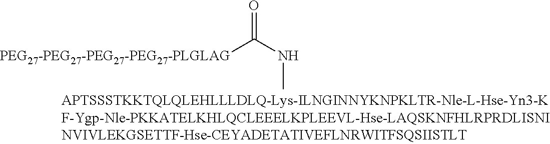 |
| 18 | CMP-143 | 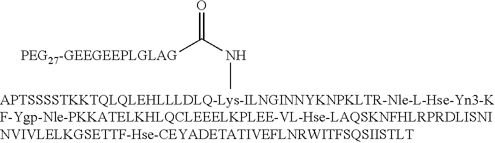 |
| 19 | CMP-144 | 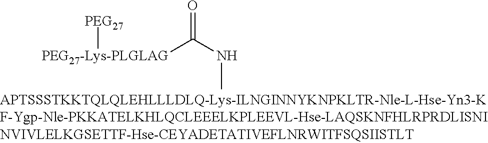 |
| 20 | CMP-145 | 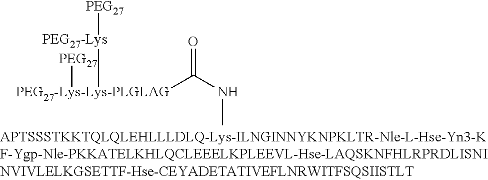 |
| 21 | CMP-146 | 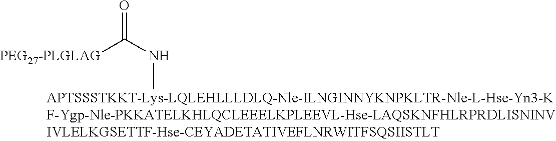 |
| 22 | CMP-147 | 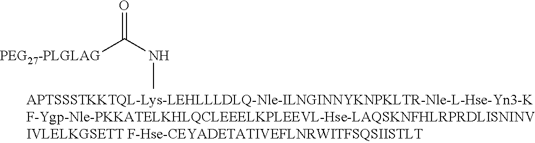 |
| 23 | CMP-148 | 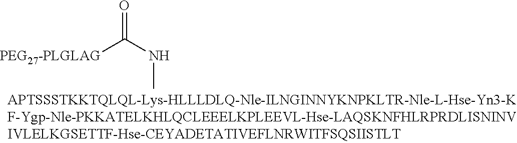 |
| 24 | CMP-149 | 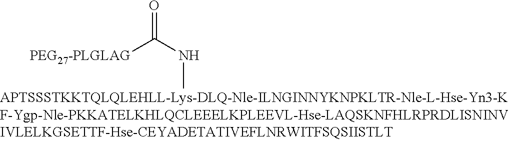 |
| 25 | CMP-150 | 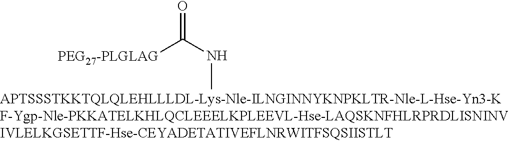 |
| 26 | CMP-162 | 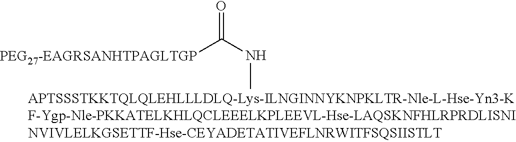 |
| 27 | CMP-133 | 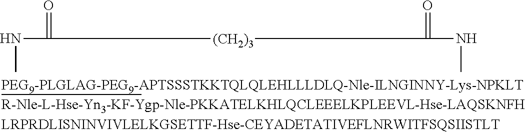 |
| 28 | CMP-134 | 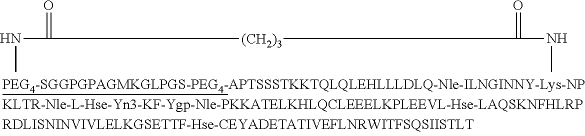 |
| 29 | CMP-135 | 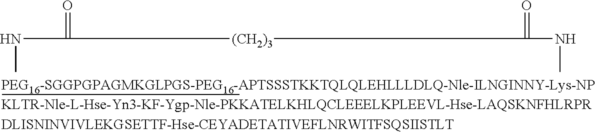 |
| 30 | CMP-140 | 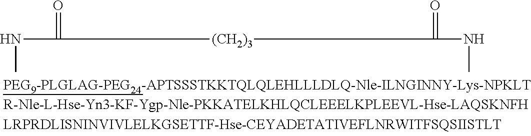 |
| 31 | CMP-152 | 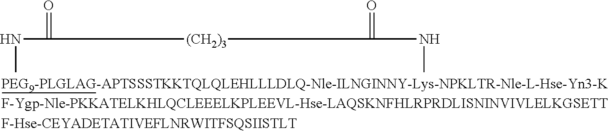 |
| 32 | CMP-153 | 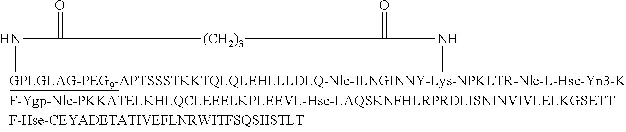 |
| 33 | CMP-136 | 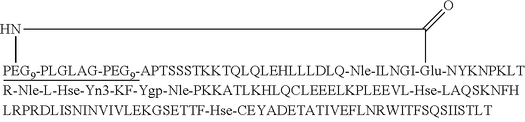 |
| 34 | CMP-137 | 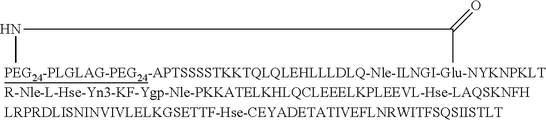 |
| 35 | CMP-138 | 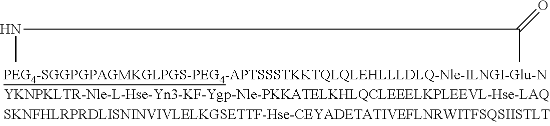 |
| 36 | CMP-139 | 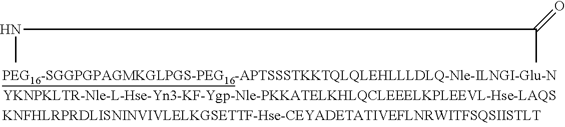 |
| 37 | CMP-154 | 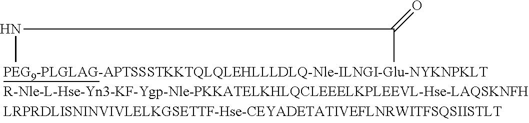 |
| 38 | CMP-155 | 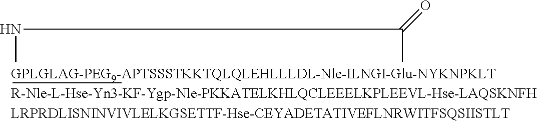 |
| 39 | CMP-156 | 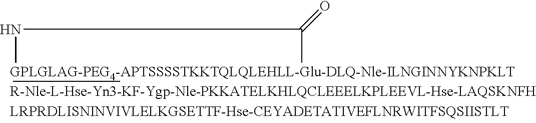 |
| 40 | CMP-157 | 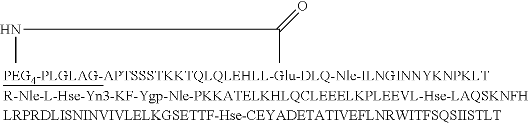 |
| 41 | CMP-158 | 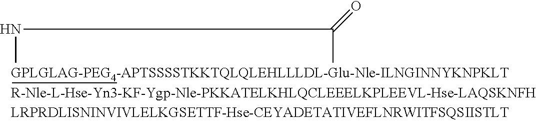 |
| 42 | CMP-159 | 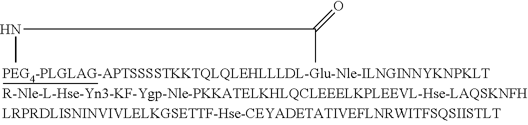 |
| 43 | CMP-160 | 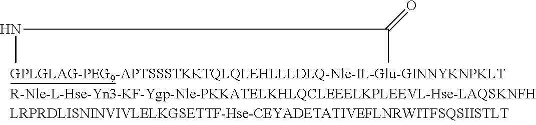 |
| 44 | CMP-161 | 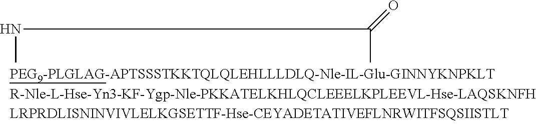 |
| 45 | CMP-162 | 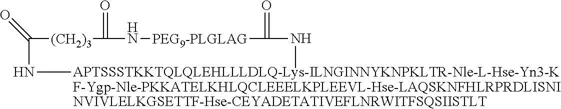 |
| 46 | CMP-163 | 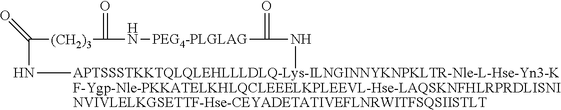 |
| 47 | CMP-164 | 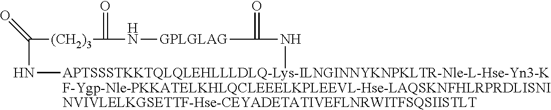 |
| 48 | CMP-165 | 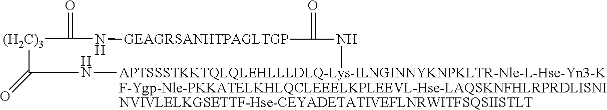 |
| 49 | CMP-166 | 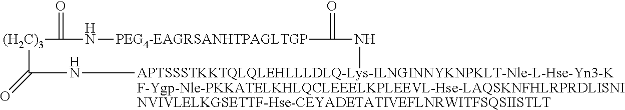 |
| 50 | CMP-167 | 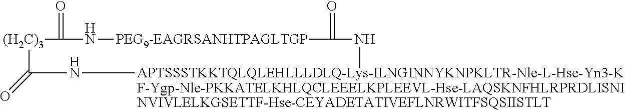 |
| 51 | CMP-168 | 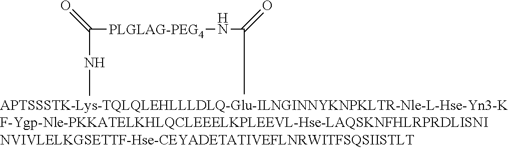 |
| 52 | CMP-169 | 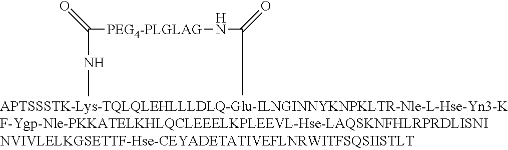 |
| 53 | CMP-130 | APTSSSTKKTQLQLE-Dab-LLLDLQ-Nle-ILNGINNYKNPKLTR-Nle-L-Hse-Yn3-KF-Ygp-Nle- |
| | | PKKATELKHLQCLEEELKPLEEVL-Hse-LAQSKNFHLRPRDLISNINVIVLELKGSETT |
| | | F-Hse-CEYADETATIVEFLNRWITFSQSIISTLT |
| 54 | CMP-131 | 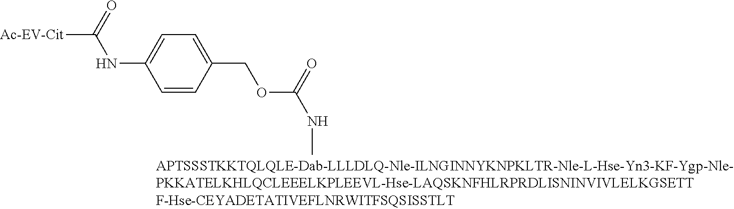 |
| 55 | CMP-132 | 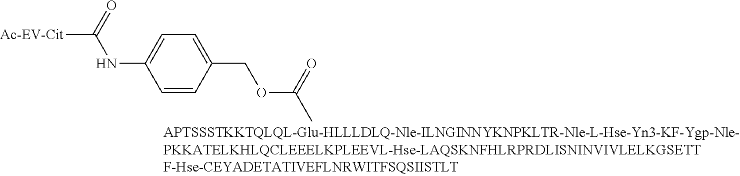 |
| 56 | CMP-300 | 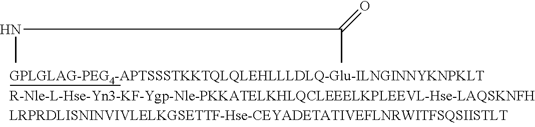 |
| 57 | CMP-301 | 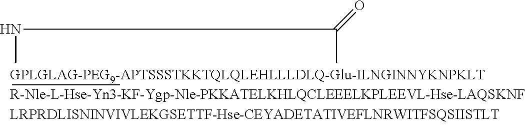 |
| 58 | CMP-302 | 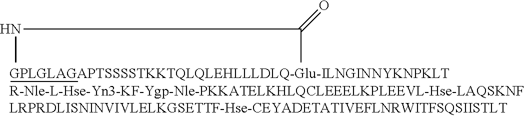 |
| 59 | CMP-303 | 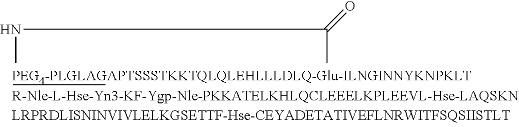 |
| 60 | CMP-304 | 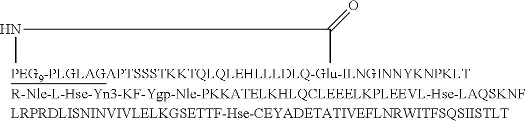 |
| 61 | CMP-305 | 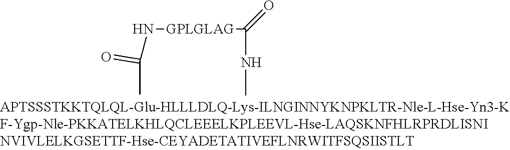 |
| 62 | CMP-310 | 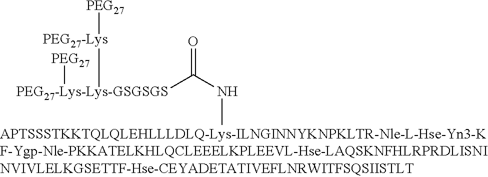 |
| 63 | CMP-311 | 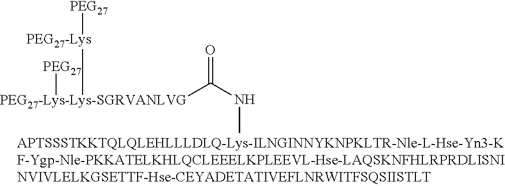 |
| 64 | CMP-312 | 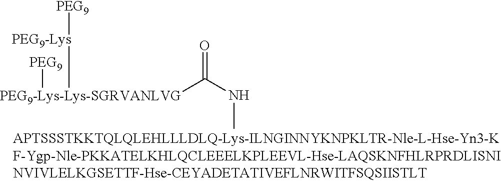 |
| 65 | CMP-313 | 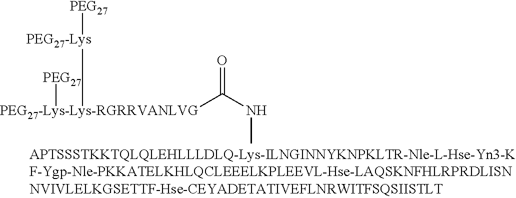 |
| 66 | CMP-314 | 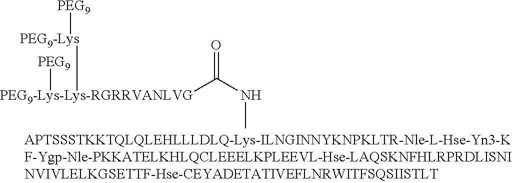 |
| 67 | CMP-315 | 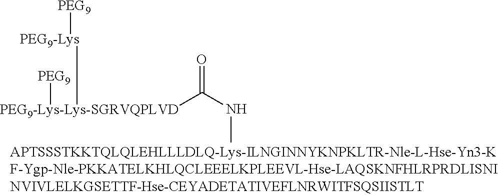 |
| 68 | CMP-316 | 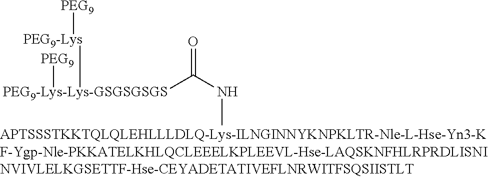 |
| 69 | CMP-317 | 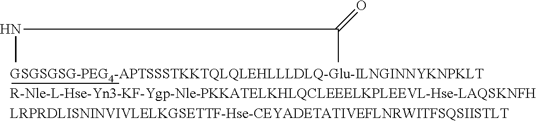 |
| 70 | CMP-318 | 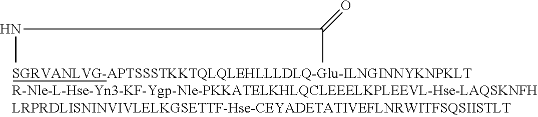 |
| 71 | CMP-319 | 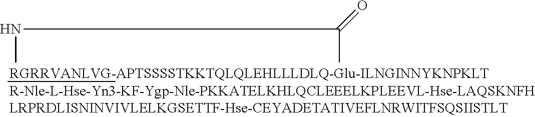 |
| 72 | CMP-320 | 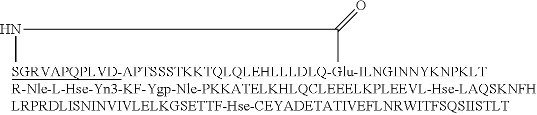 |
| 73 | CMP-321 | 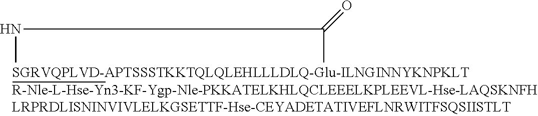 |
| 74 | CMP-322 | 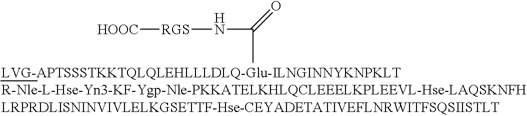 |
| 75 | CMP-323 | 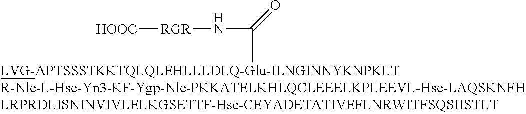 |
| 76 | CMP-324 | 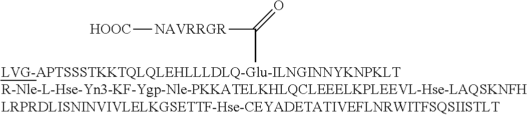 |
| 77 | CMP-325 | 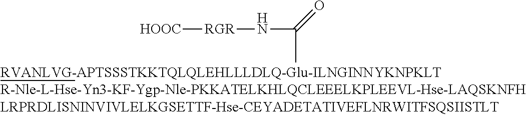 |
| 78 | CMP-326 | 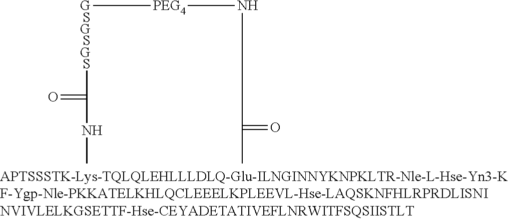 |
| 79 | CMP-327 | 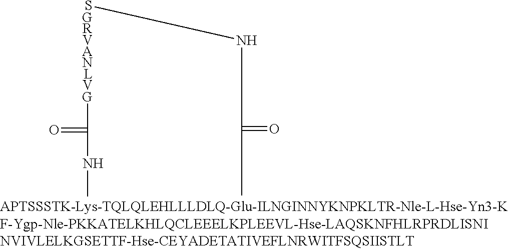 |
| 80 | CMP-328 | 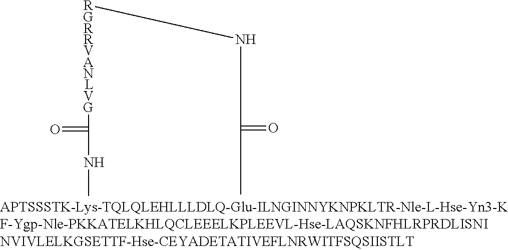 |
| 81 | CMP-329 | 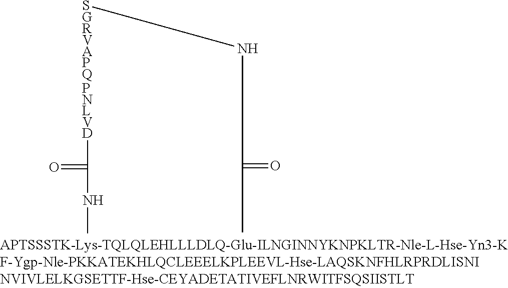 |
| 82 | CMP-330 | |
| 83 | CMP-331 | |

In Table 4 above, Ne is a norleucine residue and Hse is a homoserine residue, Dab is 2,4-diamino butyric acid, Cit is a citrulline residue, Yn3 is a tyrosine residue modified with an azide-capped PEG9 group (see below), and Ygp is a tyrosine residue modified with an amino-capped PEG8 group (see below).

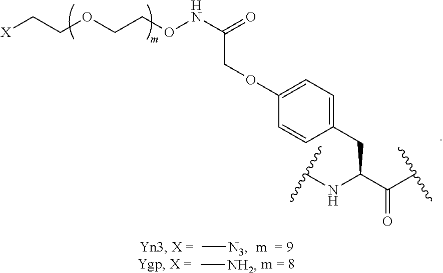

In some embodiments, the cleavable peptide of the activatable IL-2 polypeptide comprises a protease cleavable peptide. In some embodiments, the cleavable peptide of IL-2 is cleavable by a kallikrein, thrombin, chymase, carboxyprotease A, and elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof.

In some embodiments, the cleavable peptide of the activatable IL-2 polypeptide is cleavable by multiple proteases. In some embodiments, the cleavage of the cleavable peptide leaves 1, 2, 3, 4, 5, 6, 7, 8, 9, or 10 amino acids attached to the side chain of the amino acid residue of the activatable IL-2 to which the cleavable peptide is attached. In some embodiments, the cleavable peptide of the activatable IL-2 comprises an amino acid sequence having at least about 80%, at least about 90%, or at least about 100% identity to a sequence set forth in any one of Table 1 Table 2 or Table 3A/B. In some embodiments, the C-terminus of the cleavable peptide is attached to the side chain of the amino acid residue of the activatable IL-2. In some embodiments, the amino acid residue of the activatable IL-2 to which the cleavable peptide is attached is lysine, glutamate, glutamine, aspartate, asparagine, tyrosine, serine, threonine, cysteine, or an unnatural amino acid (e.g., 2,4-diaminobutyric acid). In some embodiments, the amino acid residue in the activatable IL-2 polypeptide to which the cleavable peptide is attached is a lysine or glutamate. In some embodiments, the activatable IL-2 polypeptide to which the cleavable peptide is attached is a lysine. In some embodiments, the amino acid residue in the activatable IL-2 polypeptide to which the cleavable peptide is attached is a glutamate. In some embodiments, the amino acid residue in the activatable IL-2 to which the cleavable peptide is attached is substituted relative to the corresponding residue in SEQ ID NO: 1. In some embodiments, the cleavable peptide is attached to a residue on the activatable IL-2 which contacts the IL-2 receptor beta subunit or the Il-2 receptor gamma subunit during binding to the IL-2 receptor. In some embodiments, the cleavable peptide is attached to a residue selected from residues 9, 11, 13, 15, 16, 19, 20, 22, 23, 26, 29, 32, 84, 88, 91, 123, 126, and 129 of the IL-2 polypeptide, wherein residue position numbering is based on SEQ ID NO: 1 as a reference sequence. In some embodiments, the cleavable peptide is attached to residue 9, 11, 13, 15, 16, 19, 22, 23, 29, or 32 of the activatable IL-2 polypeptide, wherein residue position numbering is based on SEQ ID NO: 1 as the reference sequence.

In some embodiments, the cleavable moiety is attached to the activatable IL-2 polypeptide at an additional point of attachment. In some embodiments, the additional point of attachment is to the N-terminus of the activatable IL-2 polypeptide. In some embodiments, the additional point of attachment is to another amino acid residue of the IL-2 polypeptide. In some embodiments, the additional point of attachment is to residue of the IL-2 polypeptide, wherein residue numbering is based on SEQ ID NO: 1 as a reference sequence. In some embodiments, the cleavable peptide is attached to the side chain of the amino acid residue of the activatable IL-2 through a linking group. In some embodiments, the IL-2 polypeptide exhibits reduced binding to the IL-2 receptor alpha subunit compared to wild type IL-2.

In some embodiments, the IL-2 comprises at least one modification that reduces the affinity of the IL-2 receptor alpha compared to the wild type IL-2. In some embodiments, IL-2 polypeptide comprises at least one polymer covalently attached to a residue selected from residues 35, 37, 38, 41, 42, 43, 44, 45, 60, 61, 62, 64, 65, 68, 69, 71, 72, 104, 105, and 107, wherein residue position numbering is based on SEQ ID NO: 1 as a reference sequence, wherein the polymer acts as a blocking moiety. In some embodiments, the IL-2 polypeptide comprises at least one polymer covalently attached to a residue selected from residue 42 and 45, wherein residue position numbering is based on SEQ ID NO: 1 as a reference sequence. In some embodiments, the IL-2 polypeptide comprises polymers covalently attached at residues 42, and 45, wherein residue position numbering is based on SEQ ID NO: 1 as a reference sequence.

In some embodiments, the IL-2 polypeptide is synthetic. In some embodiments, the IL-2 polypeptide comprises an amino acid sequence having at least about 80%, at least about 85%, at least about 90%, at least about 95% sequence identity to SEQ ID NO: 3.

In some embodiments, the cleavable peptide is attached to an additional moiety. In some embodiments, cleavage of the cleavable moiety releases the additional moiety from the IL-2 polypeptide. In some embodiments, the activatable IL-2 polypeptide is attached to an additional polypeptide.

In some embodiments, the activatable IL-2 is attached to an additional polypeptide. In some embodiments, the additional polypeptide is an antibody or antigen binding fragment thereof. In some embodiments, the additional polypeptide comprises an anti-PD-1 antibody or antigen binding fragment thereof.

### Pharmaceutical Compositions

In some embodiments, provided herein is a pharmaceutical composition comprising the activatable protein and one or more pharmaceutically acceptable solvents. In some embodiments, the pharmaceutical composition comprises activatable IL-2 and a pharmaceutically acceptable carrier.

### Methods of Treatment

In some embodiments, a method of treating cancer in a subject comprises administering to a subject a pharmaceutically effective amount of the activatable protein. In some embodiments, a method of treating cancer in a subject comprises administering to a subject a pharmaceutically effective amount of an activatable IL-2 polypeptide or a pharmaceutical composition containing an activatable IL-2.

In some embodiments, the cancer is a solid cancer. In some embodiments, the solid cancer is an adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

In some embodiments, provided herein is an isolated polypeptide comprising a cleavable peptide having an amino acid sequence having at least about 80%, at least about 90%, or 100% identity to a sequence set forth in any one of Table 1, Table 2 or Table 3A/B, wherein the cleavable peptide is attached to a side chain of an amino acid residue of the isolated polypeptide.

In some embodiments, the cleavable peptide has an amino acid sequence having at least about 80%, at least about 90%, or 100% identity to a sequence set forth in Table 2. In some embodiments, the artificial cleavable peptide is attached to a side chain of an amino acid residue of the artificial polypeptide.

### Methods of Manufacturing

In some embodiments described herein, is a method of making an activatable protein. In some embodiments the activated protein is a modified IL-2 polypeptide. In some embodiments, described herein, is a method of making a modified IL-2 polypeptide comprising synthesizing two or more fragments of the modified IL-2 polypeptide and ligating the fragments. In some embodiments, described herein, is a method of making a modified IL-2 polypeptide comprising a.) synthesizing two or more fragments of the modified IL-2 polypeptide, b.) ligating the fragments; and c.) folding the ligated fragments. Examples of methods synthesizing IL-2 polypeptides can also be found in, for example, at least PCT Publication No WO2021140416A2, US Patent Application Publication No US20190023760A1, and Asahina et al., Angew. Chem. Int. Ed. 2015, 54, 8226-8230, each of which is incorporated by reference as if set forth herein in its entirety.

In some embodiments, the two or more fragments of the modified IL-2 polypeptide are synthesized chemically. In some embodiments, the two or more fragments of the modified IL-2 polypeptide are synthesized by solid phase peptide synthesis. In some embodiments, the two or more fragments of the modified IL-2 polypeptide are synthesized on an automated peptide synthesizer.

In some embodiments, the modified IL-2 polypeptide is ligated from 2, 3, 4, 5, 6, 7, 8, 9, 10, or more peptide fragments. In some embodiments, the modified peptide is ligated from 2 peptide fragments. In some embodiments, the modified IL-2 polypeptide is ligated from 3 peptide fragments. In some embodiments, the modified IL-2 polypeptide is ligated from 4 peptide fragments. In some embodiments, the modified IL-2 polypeptide is ligated from 2 to 10 peptide fragments.

In some embodiments, the two or more fragments of the modified IL-2 polypeptide are ligated together. In some embodiments, three or more fragments of the modified IL-2 polypeptide are ligated in a sequential fashion. In some embodiments, three or more fragments of the modified IL-2 polypeptide are ligated in a one-pot reaction.

In some embodiments, ligated fragments are folded. In some embodiments, folding comprises forming one or more disulfide bonds within the modified IL-2 polypeptide. In some embodiments, the ligated fragments are subjected to a folding process. In some embodiments, the ligated fragments are folding using methods well known in the art. In some embodiments, the ligated polypeptide or the folded polypeptide are further modified by attaching one or more polymers thereto. In some embodiments, the ligated polypeptide or the folded polypeptide are further modified by PEGylation.

In some embodiments, the modified IL-2 polypeptide is synthetic. In some embodiments, the modified IL-2 polypeptide is recombinant.

In some embodiments, described herein is a host cell comprising a modified IL-2 polypeptide. In some embodiments, the host cell is a prokaryotic cell or a eukaryotic cell. In some embodiments, the host cell is a mammalian cell, an avian cell, and an insect cell. In some embodiments, the host cell is a CHO cell, a COS cell, or a yeast cell.

In some embodiments, described herein is a method of producing a modified IL-2 polypeptide, wherein the method comprises expressing the modified IL-2 polypeptide in a host cell. In some embodiments, the host cell is a prokaryotic cell or a eukaryotic cell. In some embodiments, the host cell is a mammalian cell, an avian cell, and an insect cell. In some embodiments, the host cell is a CHO cell, a COS cell, or a yeast cell.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### Example 1: Synthesis of Cleavable Peptides

### Rationale:

Peptides bearing different amino acid composition were designed for testing of cleavage efficiency using a FRET based assay (Table 3). Each cleavable peptide has a fluorophore on one end (N—/C-terminal), a quencher on the other (N—/C-terminal) and a sequence recognized by one or more proteases interspersed therein. The peptides described in the Examples (LNK001-LNK081) below have the sequences indicated in Tables 3A and 3B supra with a C-terminal Tyr(3-NO₂) residue and an N-terminal Abz group acting as a FRET pair to assess cleavage of the peptide.

### Cleavable Peptide Synthesis:

Individual peptides are synthesized on an automated peptide synthesizer using the methods described below.

Materials and solvents: Fmoc-amino acids with suitable side chain protecting groups for Fmoc-SPPS, resins polyethylene glycol derivatives used for peptide functionalization and reagents were commercially available and were used without further purification. HPLC grade CH₃CN was used for analytical and preparative RP-HPLC purification. The following Fmoc-amino acids with side-chain protecting groups were used: Fmoc-Ala-OH, Fmoc-Arg(Pbf)-OH, Fmoc-Asn(Trt)-OH, Fmoc-Asp(OtBu)-OH, Fmoc-Gln(Trt)-OH, Fmoc-Glu-OAll, Fmoc-Glu(OtBu)-OH, Fmoc-Gly-OH, Fmoc-Leu-OH, Fmoc-Lys(Boc)-OH, Fmoc-Met-OH, Fmoc-Nle-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Abz-OH and Fmoc-Tyr(3-NO₂)—OH.

### Special Building Blocks Structures

Fmoc-Tyr(3-NO₂)—OH 1

Fmoc-Abz-OH 2

Loading of Fmoc-Tyr(3-NO₂)—OH on amine-based resin: Rink-amide MBHA resin (0.57 mmol/g) was swollen in NMP for 30 min. Fmoc-deprotection was performed twice with 20% 4-Methylpiperidine in NMP v/v (40 mL/mmol resin substitution) at RT for 10 min followed by several washes with DMF. Fmoc-Tyr(3-NO₂)—OH 1 (3.00 equiv. to resin substitution) was dissolved in NMP and added to resin followed by HCTU (3 equiv.) and NMM (16 equiv.) in NMP (60 mL/mmol resin substitution). After 2 h at RT under gentle agitation, the resin was rinsed thoroughly with NMP. Capping of unreacted amines on the resin was performed by addition of a solution of 20% acetic anhydride in NMP v/v (0.1M, 10 equiv.) and NMM (0.8 M, 16 equiv.) in NMP. After 6 min under gentle agitation, the resin was washed thoroughly with NMP. Fmoc deprotection was performed twice 2 min with 4-Methylpiperidine in NMP (40 mL/mmol resin substitution) and the resin was washed thoroughly with NMP (twice), IPA (twice) and NMP (twice) (60 mL/mmol resin substitution).

Elongation of the peptide: The peptides were synthesized on an automated peptide synthesizer using Fmoc-SPPS chemistry. Double couplings of 5 min were performed with Fmoc-amino acid (8 equiv. to resin substitution), HCTU (8 equiv.) as coupling reagents and NMM (16 equiv.) in NMP (60 mL/mmol resin substitution) at RT. After the resin was treated with 20% acetic anhydride (10 equiv.) in NMP in presence of NMM (16 equiv.) for capping any unreacted free amine. Fmoc deprotection were performed twice 2 min with 4-Methylpiperidine in DMF (2×2 min, 40 mL/mmol resin substitution) and the resin was washed thoroughly with NMP (twice), IPA (twice) and NMP (twice) (60 mL/mmol resin substitution).

Coupling of Fmoc-Abz-OH: Double couplings of 1 h were performed with Fmoc-Abz-OH 2 (4 equiv. to resin substitution), HCTU (4 equiv.) as coupling reagents and NMM (8 equiv.) in NMP (40 mL/mmol resin substitution) at RT. Aft the resin was treated with 20% acetic anhydride (10 equiv.) in NMP in presence of NMM (16 equiv.) for capping any unreacted free amine for 6 min. Fmoc deprotection was performed twice 2 min with 4-Methylpiperidine in DMF (2×2 min, 40 mL/mmol resin substitution) and the resin was washed thoroughly with NMP (twice), IPA (twice) and NMP (twice) (60 mL/mmol resin substitution).

Resin cleavage and side chain deprotection of the peptides: Once the peptide synthesis was completed, the peptides were cleaved from the resin using a cleavage TFA/TIS/Water 95:2.5:2.5 v/v/v (10 mL/mmol resin substitution) at room temperature for 2 h. The resin was filtered off, and the filtrate treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v v) and analyzed by analytical RP-HPLC using C18 column (4.6×150 mm) at 50° C. The molecular weight of the product was identified using LC-MS.

Purification of the cleavable peptides: Peptides were purified by RP-HPLC on C18 column (5p m, 20×250 mm). Different gradients were applied for the different peptides. The mobile phase was MilliQ-H₂O with 0.1% TFA (v v) (Buffer A) and HPLC grade ACN with 0.1% TFA (v v) (Buffer B). The temperature was set at 50° C. UV detection was performed at 220 and 254 wavelengths. The gradient applied is summarized in the table below.

| Time | Flow | | |
|---|---|---|---|
| (min) | (ml/min) | A % | B % |
| 0 | 15 | 90 | 10 |
| 4 | 15 | 90 | 10 |
| 4.5 | 15 | 82 | 18 |
| 20 | 15 | 62 | 38 |
| 20.2 | 15 | 5 | 95 |
| 25 | 15 | 5 | 95 |

### Characterization of the Cleavable Peptides:

Peptide were analyzed by RP-HPLC coupled to ESI-MS (see methods below).

### HPLC Methods:

### Method 1:

Column: Waters XBridge C18 3.5 μm; 3×150 mm
Temperature: 50° C.
Gradient:

| Time | Flow | | |
|---|---|---|---|
| (min) | (mL/min) | % A | % B |
| 0 | 0.8 | 95 | 5 |
| 2 | 0.8 | 95 | 5 |
| 17 | 0.8 | 45 | 55 |
| 17.1 | 0.8 | 5 | 95 |
| 19 | 0.8 | 5 | 95 |
| 19.1 | 0.8 | 95 | 5 |
| 21 | 0.8 | 95 | 5 |

### Method 2:

Column: Waters XBridge C18 3.5 μm; 3×150 mm
Temperature: 50° C.
Gradient:

| Time | Flow | | |
|---|---|---|---|
| (min) | (mL/min) | % A | % B |
| 0 | 0.8 | 80 | 20 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 80 | 20 |
| 21 | 0.8 | 80 | 20 |

### Method 3:

Column: Waters XBridge C18 3.5 μm; 3×150 mm
Temperature: 50° C.
Gradient:

| Time | Flow | | |
|---|---|---|---|
| (min) | (mL/min) | % A | % B |
| 0 | 0.8 | 90 | 10 |
| 2 | 0.8 | 90 | 10 |
| 17 | 0.8 | 45 | 55 |
| 17.1 | 0.8 | 5 | 95 |
| 19 | 0.8 | 5 | 95 |
| 19.1 | 0.8 | 95 | 5 |
| 21 | 0.8 | 95 | 5 |

### Method 4:

Column: Waters XBridge C4 3.5 μm; 3×150 mm
Temperature: 50° C.
Gradient:

| Time | Flow | | |
|---|---|---|---|
| (min) | (mL/min) | % A | % B |
| 0 | 0.8 | 95 | 5 |
| 2 | 0.8 | 95 | 5 |
| 17 | 0.8 | 45 | 55 |
| 17.1 | 0.8 | 5 | 95 |
| 19 | 0.8 | 5 | 95 |
| 19.1 | 0.8 | 95 | 5 |
| 21 | 0.8 | 95 | 5 |

ESI-MS: The following data is collected for the synthesized cleavable peptides of Table 3.

TABLE 5
MS data for Cleavable Peptides LNK001 to LNK081

| | | | | | | MS | MS | |
|---|---|---|---|---|---|---|---|---|
| Linker | tR | HPLC | | Purity | Yield | (M + nH)n +/n | (M + nH)n +/n | |
| number | (min) | Method | Formula | (%) | (%) | theoretical | found | n |
| LNK001 | 11.17 | 1 | C₈₇H₁₃₉N₃₁O₂₆ | 98.5 | 22.6 | 679.4 | 679.5 | 3 |
| LNK002 | 7.54 | 2 | C₉₃H₁₄₅N₂₉O₂₅ | 90.9 | 35.6 | 1035.7 | 1036.3 | 2 |
| LNK003 | 10.69 | 1 | C₈₅H₁₃₅N₂₇O₂₄ | 95.2 | 22.1 | 640.7 | 640.9 | 3 |
| LNK004 | 8.74 | 2 | C₉₈H₁₅₂N₂₈O₂₅ | 95.5 | 20.3 | 1062.2 | 1062.3 | 2 |
| LNK005 | 7.68 | 2 | C₆₃H₉₇N₁₉O₁₈ | 91.8 | 26.0 | 1409.6 | 1409.9 | 1 |
| LNK006 | 7.52 | 2 | C₆₅H₁₀₀N₂₀O₁₉ | 92.4 | 22.9 | 1466.6 | 1466.0 | 1 |
| LNK007 | 7.71 | 2 | C₆₁H₉₄N₁₈O₁₇ | 98.0 | 28.4 | 1352.5 | 1351.8 | 1 |
| LNK008 | 7.99 | 2 | C₆₆H₁₀₂N₂₀O₂₀ | 98.0 | 16.6 | 1496.7 | 1496.1 | 1 |
| LNK009 | 10.52 | 1 | C₆₉H₁₁₀N₂₆O₁₉ | 95.9 | 27.9 | 804.9 | 805.3 | 2 |
| LNK010 | 10.54 | 1 | C₆₆H₁₀₅N₂₅O₁₈ | 97.2 | 17.6 | 769.4 | 769.0 | 2 |
| LNK011 | 11.44 | 1 | C₈₈H₁₄₄N₂₈O₂₄ | 91.7 | 12.2 | 990.1 | 990.1 | 2 |
| LNK012 | 11.28 | 1 | C₉₀H₁₄₇N₂₉O₂₅ | 92.1 | 17.7 | 679.5 | 679.2 | 3 |
| LNK013 | 10.79 | 1 | C₇₈H₁₂₆N₂₆O₂₁ | 90.5 | 17.2 | 589.0 | 589.3 | 3 |
| LNK014 | 10.62 | 1 | C₈₀H₁₂₉N₂₇O₂₂ | 95.0 | 21.0 | 911.5 | 911.6 | 2 |
| LNK015 | 7.51 | 2 | C₆₃H₉₇N₁₉O₁₈ | 90.6 | 25.9 | 705.3 | 705.6 | 2 |
| LNK016 | 11.16 | 1 | C₅₀H₇₆N₁₆O₁₅ | 91.2 | 32.4 | 571.6 | 571.6 | 2 |
| LNK017 | 10.61 | 1 | C₅₂H₇₉N₁₇O₁₆ | 93.6 | 17.9 | 1199.3 | 1198.7 | 1 |
| LNK018 | 11.96 | 1 | C₇₃H₁₁₄N₂₄O₂₀ | 93.6 | 18.7 | 824.9 | 824.6 | 2 |
| LNK019 | 10.34 | 1 | C₇₇H₁₁₇N₂₅O₂₁ | 95.5 | 14.6 | 865.5 | 865.9 | 2 |
| LNK020 | 8.58 | 2 | C₉₁H₁₅₁N₂₇O₂₃S | 91.5 | 7.6 | 675.5 | 676.0 | 3 |
| LNK021 | 10.18 | 1 | C₇₆H₁₁₅N₂₇O₂₂S | 94.8 | 16.8 | 896.5 | 896.0 | 2 |
| LNK022 | 10.31 | 1 | C₆₂H₈₈N₁₈O₁₉S | 94.6 | 28.0 | 1422.6 | 1422.9 | 1 |
| LNK023 | 11.71 | 1 | C₆₅H₁₀₃N₂₁O₁₇S | 95.9 | 39.7 | 742.4 | 742.4 | 2 |
| LNK024 | 7.66 | 2 | C₆₇H₉₈N₁₈O₁₉S | 94.3 | 44.8 | 1492.7 | 1492.7 | 1 |
| LNK025 | 11.38 | 1 | C₇₂H₁₁₆N₂₆O₁₉ | 90.2 | 25.3 | 825.9 | 826.2 | 2 |
| LNK026 | 11.86 | 1 | C₇₁H₁₀₃N₁₉O₁₈ | 92.2 | 19.5 | 756.4 | 756.3 | 2 |
| LNK027 | 11.16 | 1 | C₆₉H₁₀₂N₂₀O₁₉ | 90.4 | 33.8 | 758.9 | 758.5 | 2 |
| LNK028 | 10.64 | 1 | C₇₂H₁₁₅N₂₇O₂₀ | 89.4 | 26.4 | 840.4 | 840.8 | 2 |
| LNK029 | 8.44 | 1 | C₅₃H₈₄N₂₂O₁₄ | 80.1 | 10.4 | 627.7 | 627.9 | 2 |
| LNK030 | 12.26 | 1 | C₄₆H₆₆N₁₂O₁₅ | 93.3 | 25.6 | 514.6 | 514.3 | 2 |
| LNK031 | 8.96 | 3 | C₆₁H₉₈N₂₂O₁₆ | 94.5 | 6.7 | 698.3 | 698.4 | 2 |
| LNK032 | 8.79 | 3 | C₆₄H₁₀₃N₂₃O₁₇ | 90.4 | 24.5 | 733.8 | 733.9 | 2 |
| LNK033 | 9.02 | 3 | C₆₁H₉₈N₂₄O₁₆ | 91.3 | 28.4 | 712.3 | 712.3 | 2 |
| LNK034 | 8.64 | 3 | C₆₄H₁₀₁N₂₃O₁₈ | 90.6 | 31.9 | 740.8 | 741.5 | 2 |
| LNK035 | 9.01 | 3 | C₆₈H₁₀₂N₂₂O₁₇S | 77 | 8.1 | 766.4 | 766.4 | 2 |
| LNK036 | 9.79 | 3 | C₆₉H₁₀₄N₂₂O₁₇ | 89.8 | 25.1 | 757.4 | 757.4 | 2 |
| LNK037 | 8.98 | 3 | C₇₁H₁₀₇N₂₃O₁₉S | 89.8 | 5.6 | 809.9 | 810.0 | 2 |
| LNK038 | 9.69 | 3 | C₇₂H₁₀₉N₂₃O₁₉ | 71.8 | 28.6 | 800.9 | 801.4 | 2 |
| LNK039 | 8.79 | 3 | C₆₆H₁₀₂N₂₂O₁₈ | 86.9 | 14.4 | 746.3 | 746.4 | 2 |
| LNK040 | 8.76 | 3 | C₆₉H₁₀₇N₂₃O₂₀ | 90.7 | 16.0 | 789.9 | 790.4 | 2 |
| LNK041 | 8.42 | 3 | C₆₄H₁₀₀N₂₂O₁₉ | 85.1 | 20.5 | 741.3 | 1483.7 | 1 |
| LNK042 | 10.49 | 3 | C₆₇H₉₇N₁₉O₁₉S | 79.5 | 3.5 | 752.8 | 1504.8 | 1 |
| LNK043 | 11.34 | 3 | C₆₈H₉₉N₁₉O₁₉ | 92 | 24.6 | 743.8 | 743.8 | 2 |
| LNK044 | 10.26 | 3 | C₅₉H₈₅N₁₇O₁₇S | 80.4 | 13.8 | 1337.5 | 1337.9 | 1 |
| LNK045 | 11.14 | 3 | C₆₀H₈₇N₁₇O₁₇ | 90.3 | 16.3 | 1319.4 | 1318.9 | 1 |
| LNK046 | 9.99 | 3 | C₆₅H₉₇N₁₉O₂₀ | 90.9 | 17.4 | 1464.6 | 1464.8 | 1 |
| LNK047 | 9.98 | 3 | C₅₇H₈₅N₁₇O₁₈ | 93 | 21.8 | 1297.4 | 1297.8 | 1 |
| LNK048 | 6.38 | 3 | C₄₃H₆₅N₁₉O₁₁ | 80.5 | 9.9 | 1025.1 | 1025.3 | 1 |
| LNK049 | 10.325 | 1 | C₆₀H₉₄N₂₂O₁₆ | 96.4 | 13.9 | 690.8 | 690.9 | 2 |
| LNK050 | 10.592 | 1 | C₇₇H₁₂₂N₂₈O₂₃ | 91.6 | 16.2 | 603.7 | 603.7 | 3 |
| LNK051 | 9.542 | 1 | C₆₇H₁₀₅N₂₅O₂₀ | 90.3 | 17.1 | 527.9 | 528.1 | 3 |
| LNK052 | 5.725 | 1 | C₆₅H₁₀₂N₂₆O₁₉ | 96.7 | 5 | 518.2 | 518.3 | 3 |
| LNK053 | 9.975 | 1 | C₅₉H₈₇N₁₉O₂₁ | 89.8 | 20.3 | 700.2 | 700 | 2 |
| LNK054 | 10.742 | 1 | C₇₁H₉₉N₁₉O₂₀ | 91.8 | 11.9 | 770.3 | 770.5 | 2 |
| LNK055 | 12.175 | 1 | C₈₀H₁₁₈N₂₆O₂₀ | 95.6 | 16.3 | 589 | 589 | 3 |
| LNK056 | 10.775 | 1 | C₇₂H₁₀₀N₁₈O₁₉ | 95.4 | 8.5 | 761.9 | 761.4 | 2 |
| LNK057 | 11.358 | 1 | C₈₁H₁₁₉N₂₅O₁₉ | 91.8 | 42 | 583.3 | 583.2 | 3 |
| LNK058 | 12.642 | 1 | C₉₃H₁₃₉N₂₉O₂₃ | 96.8 | 16.7 | 678.1 | 678.1 | 3 |
| LNK059 | 11.875 | 1 | C₁₁₉H₁₈₇N₄₁O₃₀ | 96.7 | 7.2 | 669 | 668.9 | 4 |
| LNK060 | 12.092 | 1 | C₁₀₈H₁₆₇N₃₅O₂₆ | 95.4 | 8.1 | 593.9 | 594.1 | 4 |
| LNK061 | 11.192 | 1 | C₇₇H₁₀₇N₁₉O₂₁ | 90.9 | 5.6 | 545.9 | 545.7 | 3 |
| LNK062 | 11.725 | 1 | C₁₁₀H₁₆₇N₃₅O₂₈ | 98.3 | 16.2 | 607.9 | 607.9 | 4 |
| LNK063 | 12.092 | 1 | C₁₀₁H₁₄₈N₂₈O₂₈ | 97.8 | 20 | 1103.2 | 1103.1 | 2 |
| LNK064 | 6.492 | 1 | C₃₆H₄₇N₁₁O₁₈ | 91.8 | 12 | 922.8 | 922 | 1 |
| LNK065 | 9.842 | 1 | C₅₀H₆₅N₁₁O₁₉ | 93.5 | 16.6 | 563.1 | 563.1 | 2 |
| LNK066 | 8.808 | 1 | C₄₆H₆₄N₁₂O₁₉ | 92.6 | 12.8 | 545.9 | 545.5 | 2 |
| LNK067 | 7.942 | 1 | C₅₁H₆₅N₁₃O₂₀ | 90.8 | 7.7 | 591.1 | 591.3 | 2 |
| LNK068 | 9.758 | 1 | C₅₂H₆₈N₁₂O₁₉ | 96.3 | 15.2 | 583.6 | 583.5 | 2 |
| LNK069 | 8.092 | 1 | C₄₉H₆₂N₁₂O₂₀ | 89.2 | 9.4 | 570.6 | 570.6 | 2 |
| LNK070 | 10.375 | 1 | C₅₂H₇₀N₁₂O₂₀ | 94.8 | 5.6 | 592.6 | 592.4 | 2 |
| LNK071 | 9.192 | 1 | C₅₄H₇₇N₁₃O₁₇ | 88.3 | 7.5 | 591.1 | 591.1 | 2 |
| LNK072 | 10.708 | 1 | C₅₄H₇₇N₁₃O₁₆ | 89.7 | 10.8 | 583.1 | 583.2 | 2 |
| LNK073 | 9.742 | 4 | C₅₈H₈₃N₁₉O₁₄ | 96.6 | 22.1 | 635.8 | 636.2 | 2 |
| LNK074 | 9.975 | 4 | C₅₆H₈₂N₁₈O₁₅ | 91.0 | 15.4 | 624.3 | 624.1 | 2 |
| LNK075 | 9.858 | 1 | C₅₂H₇₅N₁₇O₁₆ | 93.1 | 41.3 | 597.8 | 597.9 | 2 |
| LNK076 | 9.725 | 4 | C₅₅H₇₂N₁₄O₁₇ | 90.4 | 26.3 | 601.3 | 601.4 | 2 |
| LNK077 | 8.592 | 1 | C₅₁H₇₆N₁₆O₁₅ | 95.0 | 17.3 | 577.3 | 577.7 | 2 |
| LNK078 | 10.108 | 1 | C₅₁H₇₅N₁₅O₁₅ | 95.3 | 23.4 | 569.8 | 570.1 | 2 |
| LNK079 | 9.025 | 1 | C₅₀H₆₀N₁₂O₁₈ | 95.7 | 33.1 | 559.2 | 559.4 | 2 |
| LNK080 | 8.758 | 1 | C₄₉H₆₇N₁₅O₁₇ | 90.2 | 25.2 | 569.7 | 570.3 | 2 |
| LNK081 | 10.058 | 1 | C₄₈H₆₄N₁₂O₁₈ | 95.0 | 18.2 | 1097.4 | 1097.5 | 1 |

### Example 2

### Cleavage Efficiency of Cleavable Peptides

### Rationale:

In order to find a cleavable peptide with desirable cleavage properties from specific proteases (e.g. Matriptase, uPA and MMPs), different peptides bearing different amino acid composition were synthesized (as seen in Example 1). Each peptide carried a fluorophore on one end (N—/C-terminal) and a quencher on the other (N—/C-terminal). After successful cleavage, the fluorophore and quencher are separated thereby increasing the fluorescence intensity of the sample, thus allowing for the monitoring of the cleavage kinetic.

### Method

Peptides were incubated either with uPA (R&D System, 1310-SE), matriptase (R&D Systems, 3946-SEB), MMP-2 (SIGMA, PF023), MMP-7 (SIGMA, CC1059), or MMP-9 (SIGMA, PF024) at a final concentration of 200 uM and 1 ug/ml for the linker and the proteases, respectively, except for Kallikrein-3 which was used at 2 μg/ml. Cleavage was performed at 37° C. under shaking conditions in buffer A (50 mM TRIS, 0.01% Tween20, pH 8.5), buffer B (50 mM TRIS, 50 mM NaCl, 0.01% Tween20, pH 9.0), or buffer C (25 mM TRIS, 10 mM CaCl₂), 0.05% Brij25, pH 7.5) for uPA, matriptase and MMPs, respectively. Kallikrein-3 was tested in a buffer of 50 mM TRIS, 10 mM CaCl₂), 1 M NaCl, pH 8. For some peptides, cleavage was also performed in 25 mM TRIS, 10 mM CaCl₂, 0.05% Brij25 titrated at either pH 7.5 or pH 6.5 with MMP7 (SIGMA, CC1059) and/or MMP9 (SIGMA, PF024). A control without enzymes was incubated in the same conditions. The digestion was analyzed by plate reader (PerkinElmer, EnSpire) at an excitation/emission ratio of 360/450. Digestions were performed at 37° C. Every 60 seconds, the samples were shaken for 5 seconds and a read was taken for a total of 150 reads. Data were analyzed by GraphPad Prism 9.

### Results

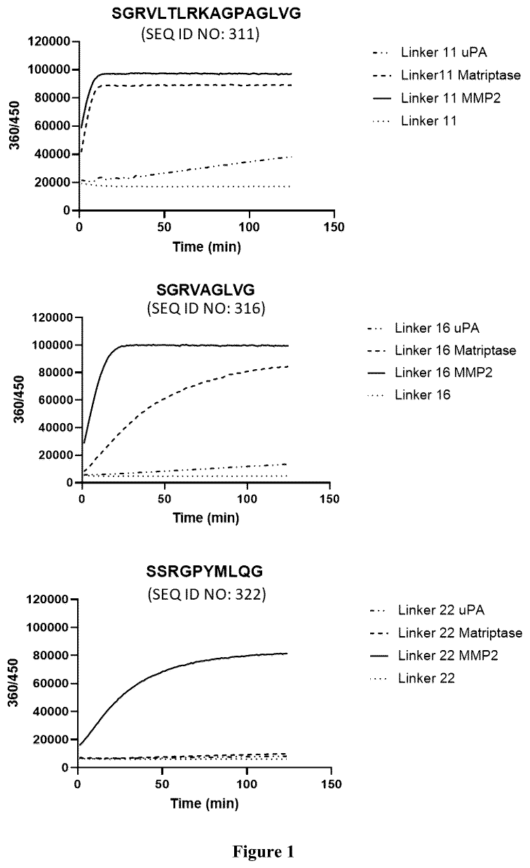

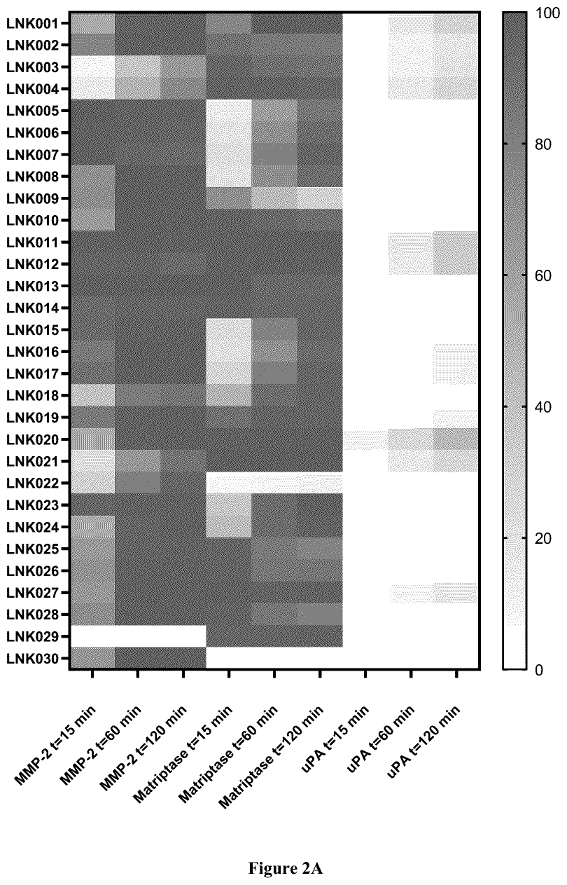

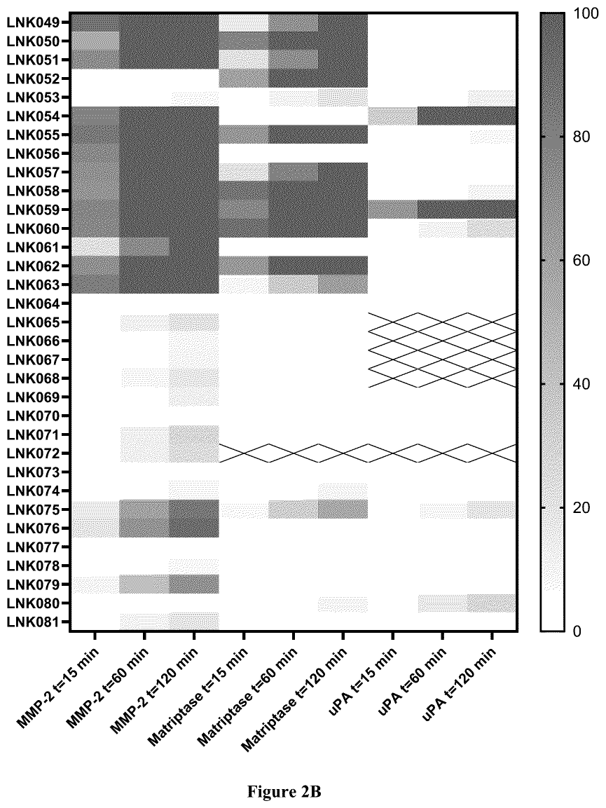

All the peptides are evaluated and their cleavage kinetic assessed. Protease cleavage efficiency for peptides LNK001 to LNK030 is provided in Table 6. Representative cleavage data for select peptides is also shown in FIGS. 1, 2A, and 2B.

TABLE 6
Cleavage efficiency of LNK001 to LNK030 with different proteases

| | % | % | % | % | % | % | % | % | % |
|---|---|---|---|---|---|---|---|---|---|
| | cleavage | cleavage | cleavage | cleavage | cleavage | cleavage | cleavage | cleavage | cleavage |
| | MMP-2 | MMP-2 | MMP-2 | Matriptase | Matriptase | Matriptase | uPA | uPA | uPA |
| | t = 15 | t = 60 | t = 120 | t = 15 | t = 60 | t = 120 | t = 15 | t = 60 | t = 120 |
| Linker | min | min | min | min | min | min | min | min | min |
| LNK001 | 54 | 98 | 99 | 77 | 99 | 99 | 4 | 17 | 30 |
| LNK002 | 77 | 99 | 98 | 89 | 85 | 84 | 5 | 10 | 18 |
| LNK003 | 8 | 38 | 65 | 96 | 91 | 88 | 3 | 10 | 20 |
| LNK004 | 16 | 50 | 74 | 98 | 98 | 95 | 6 | 16 | 27 |
| LNK005 | 100 | 98 | 97 | 15 | 63 | 86 | 0 | 0 | 1 |
| LNK006 | 98 | 98 | 96 | 19 | 71 | 92 | 0 | 0 | 1 |
| LNK007 | 99 | 95 | 94 | 23 | 79 | 97 | 0 | 0 | 1 |
| LNK008 | 71 | 99 | 99 | 20 | 72 | 92 | 1 | 1 | 2 |
| LNK009 | 72 | 99 | 99 | 71 | 45 | 31 | 0 | 1 | 3 |
| LNK010 | 64 | 99 | 99 | 98 | 94 | 91 | 0 | 1 | 2 |
| LNK011 | 98 | 99 | 98 | 99 | 99 | 99 | 6 | 17 | 35 |
| LNK012 | 97 | 97 | 94 | 99 | 99 | 98 | 0 | 15 | 34 |
| LNK013 | 99 | 99 | 100 | 99 | 94 | 94 | 0 | 2 | 6 |
| LNK014 | 93 | 97 | 97 | 97 | 95 | 95 | 0 | 2 | 7 |
| LNK015 | 94 | 99 | 98 | 25 | 78 | 96 | 0 | 0 | 1 |
| LNK016 | 85 | 99 | 99 | 22 | 70 | 92 | 2 | 6 | 12 |
| LNK017 | 90 | 99 | 99 | 29 | 81 | 96 | 1 | 6 | 12 |
| LNK018 | 40 | 83 | 87 | 49 | 92 | 97 | 0 | 0 | 0 |
| LNK019 | 84 | 99 | 99 | 90 | 97 | 97 | 4 | 6 | 10 |
| LNK020 | 55 | 98 | 99 | 98 | 99 | 99 | 11 | 26 | 45 |
| LNK021 | 23 | 66 | 88 | 100 | 100 | 100 | 5 | 16 | 27 |
| LNK022 | 31 | 81 | 96 | 8 | 9 | 11 | 0 | 1 | 3 |
| LNK023 | 96 | 98 | 98 | 38 | 92 | 99 | 2 | 2 | 3 |
| LNK024 | 55 | 97 | 99 | 44 | 92 | 98 | 1 | 0 | 1 |
| LNK025 | 64 | 97 | 98 | 97 | 84 | 77 | 0 | 1 | 2 |
| LNK026 | 69 | 98 | 98 | 97 | 86 | 86 | 0 | 1 | 3 |
| LNK027 | 66 | 99 | 99 | 99 | 97 | 98 | 3 | 9 | 17 |
| LNK028 | 72 | 99 | 99 | 97 | 86 | 79 | 0 | 1 | 2 |
| LNK029 | 0 | 0 | 0 | 96 | 96 | 98 | 0 | 0 | 1 |
| LNK030 | 67 | 100 | 99 | 0 | 0 | 0 | 0 | 0 | 0 |
| LNK049 | 89 | 100 | 100 | 16 | 70 | 100 | 0 | 0 | 0 |
| LNK050 | 54 | 100 | 100 | 78 | 100 | 100 | 1 | 3 | 6 |
| LNK051 | 73 | 100 | 100 | 22 | 72 | 100 | 0 | 0 | 0 |
| LNK052 | 1 | 2 | 4 | 60 | 100 | 100 | 0 | 0 | 0 |
| LNK053 | 2 | 5 | 8 | 3 | 11 | 20 | 1 | 5 | 12 |
| LNK054 | 78 | 100 | 100 | 0 | 0 | 0 | 33 | 100 | 100 |
| LNK055 | 85 | 100 | 100 | 67 | 100 | 100 | 2 | 4 | 8 |
| LNK056 | 75 | 100 | 100 | 0 | 0 | 0 | 0 | 0 | 0 |
| LNK057 | 71 | 100 | 100 | 22 | 78 | 100 | 0 | 0 | 0 |
| LNK058 | 68 | 100 | 100 | 88 | 100 | 100 | 1 | 5 | 10 |
| LNK059 | 77 | 100 | 100 | 76 | 100 | 100 | 67 | 100 | 100 |
| LNK060 | 76 | 100 | 100 | 90 | 100 | 100 | 2 | 11 | 25 |
| LNK061 | 23 | 74 | 100 | 0 | 0 | 0 | 0 | 0 | 0 |
| LNK062 | 71 | 100 | 100 | 66 | 100 | 100 | 0 | 0 | 0 |
| LNK063 | 82 | 100 | 100 | 9 | 35 | 62 | 0 | 0 | 0 |
| LNK064 | 0 | 0 | 0 | 0 | 0 | 0 | 0 | 0 | 0 |
| LNK065 | 3 | 12 | 21 | 0 | 0 | 0 | n.d | n.d. | n.d. |
| LNK066 | 1 | 5 | 9 | 0 | 0 | 0 | n.d. | n.d. | n.d. |
| LNK067 | 1 | 5 | 10 | 0 | 0 | 0 | n.d | n.d. | n.d |
| LNK068 | 2 | 9 | 18 | 0 | 0 | 0 | n.d | n.d. | n.d. |
| LNK069 | 1 | 6 | 13 | 0 | 0 | 0 | 0 | 0 | 0 |
| LNK070 | 0 | 0 | 0 | 0 | 0 | 0 | 0 | 0 | 0 |
| LNK071 | 1 | 13 | 28 | 0 | 0 | 0 | 0 | 0 | 0 |
| LNK072 | 1 | 13 | 25 | n.d | n.d | n.d | n.d | n.d | n.d |
| LNK073 | 1 | 4 | 6 | 1 | 3 | 5 | 0 | 0 | 0 |
| LNK074 | 1 | 5 | 9 | 2 | 6 | 10 | 0 | 0 | 0 |
| LNK075 | 16 | 60 | 86 | 8 | 33 | 56 | 2 | 9 | 19 |
| LNK076 | 19 | 69 | 92 | 0 | 0 | 0 | 0 | 0 | 0 |
| LNK077 | 3 | 4 | 6 | 0 | 0 | 0 | 0 | 0 | 0 |
| LNK078 | 3 | 6 | 8 | 0 | 0 | 0 | 0 | 0 | 0 |
| LNK079 | 10 | 42 | 70 | 0 | 0 | 0 | 0 | 0 | 0 |
| LNK080 | 1 | 3 | 5 | 2 | 6 | 10 | 4 | 17 | 28 |
| LNK081 | 3 | 11 | 19 | 0 | 0 | 0 | 0 | 0 | 0 |

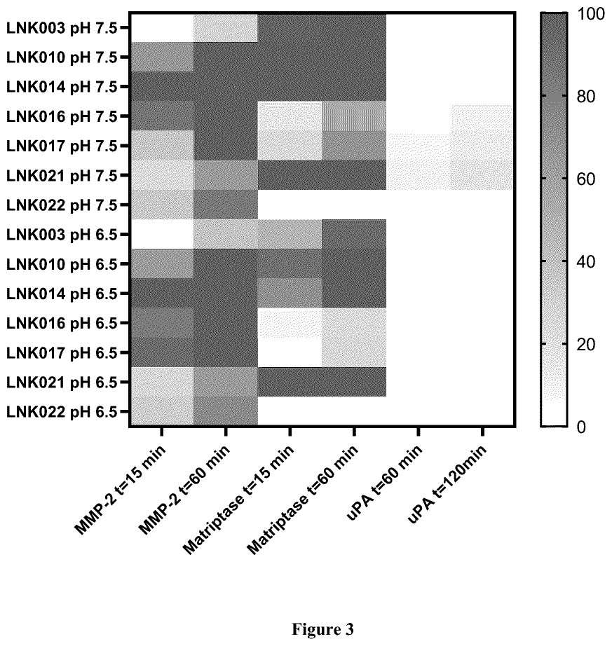

Selected peptides were further titrated for cleavage efficiency at pH 7.5 and pH 6.5. See Table 7. Also see FIG. 3.

TABLE 7
Cleavage efficiency of selected peptides at pH 7.5 and pH 6.5.

| | | % | % | % | % | % | % |
|---|---|---|---|---|---|---|---|
| | | cleavage | cleavage | cleavage | cleavage | cleavage | cleavage |
| | | MMP-2 | MMP-2 | Matriptase | Matriptase | uPA | uPA |
| | | t = 15 | t = 60 | t = 15 | t = 60 | t = 60 | t = 120 |
| pH | Linker | min | min | min | min | min | min |
| pH 7.5 | LNK003 | 6 | 28 | 98 | 100 | 0 | 0 |
| | LNK010 | 66 | 100 | 100 | 100 | 0 | 0 |
| | LNK014 | 100 | 100 | 100 | 100 | 0 | 0 |
| | LNK016 | 87 | 100 | 17 | 54 | 4 | 10 |
| | LNK017 | 37 | 100 | 25 | 68 | 7 | 13 |
| | LNK021 | 24 | 64 | 100 | 100 | 10 | 19 |
| | LNK022 | 36 | 84 | 0 | 0 | 0 | 0 |
| pH 6.5 | LNK003 | 5 | 39 | 48 | 93 | 0 | 0 |
| | LNK010 | 63 | 100 | 90 | 100 | 0 | 0 |
| | LNK014 | 100 | 100 | 70 | 100 | 0 | 0 |
| | LNK016 | 84 | 100 | 8 | 24 | 0 | 0 |
| | LNK017 | 93 | 100 | 5 | 26 | 0 | 0 |
| | LNK021 | 26 | 64 | 100 | 100 | 0 | 0 |
| | LNK022 | 32 | 74 | 0 | 0 | 0 | 0 |

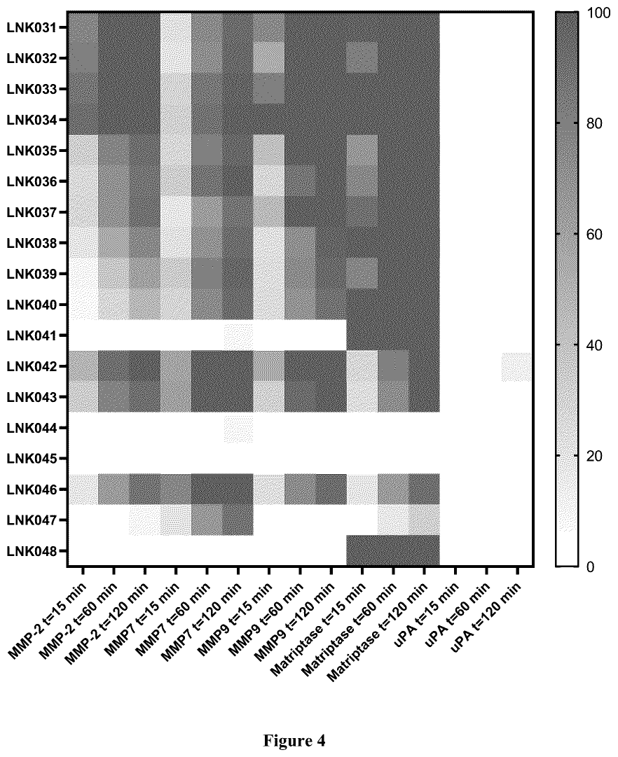

In addition to the above, certain peptides were further tested for cleavage efficiency with MMPs matriptase, and uPA under standard conditions. See Table 8 and FIG. 4

TABLE 8
Cleavage efficiency of selected peptides by MMP.

| | | % | % | % | % | % | % | % |
|---|---|---|---|---|---|---|---|---|
| | | cleavage | cleavage | cleavage | cleavage | cleavage | cleavage | cleavage |
| | % | MMP-2 | MMP-2 | MMP-7 | MMP-7 | MMP-7 | MMP-9 | MMP-9 |
| Linker | cleavage | t = 60 min | t = 120 min | t = 15 min | t = 60 min | t = 120 min | t = 15 min | t = 60 min |
| LNK031 | 77 | 100 | 100 | 22 | 74 | 93 | 75 | 100 |
| LNK032 | 81 | 100 | 100 | 20 | 72 | 95 | 53 | 100 |
| LNK033 | 87 | 100 | 100 | 30 | 86 | 98 | 81 | 100 |
| LNK034 | 92 | 100 | 100 | 32 | 89 | 100 | 100 | 100 |
| LNK035 | 33 | 77 | 91 | 28 | 80 | 95 | 42 | 100 |
| LNK036 | 27 | 70 | 88 | 34 | 88 | 100 | 28 | 87 |
| LNK037 | 28 | 70 | 89 | 19 | 63 | 88 | 46 | 100 |
| LNK038 | 19 | 55 | 76 | 22 | 69 | 92 | 23 | 71 |
| LNK039 | 10 | 38 | 61 | 35 | 80 | 96 | 25 | 74 |
| LNK040 | 8 | 28 | 47 | 29 | 74 | 93 | 25 | 69 |
| LNK041 | 0 | 0 | 0 | 1 | 4 | 9 | 0 | 1 |
| LNK042 | 49 | 91 | 100 | 59 | 100 | 100 | 55 | 100 |
| LNK043 | 34 | 78 | 91 | 61 | 100 | 100 | 32 | 93 |
| LNK044 | 0 | 0 | 1 | 1 | 4 | 9 | 0 | 1 |
| LNK045 | 0 | 0 | 0 | 0 | 0 | 0 | 0 | 0 |
| LNK046 | 19 | 64 | 88 | 77 | 100 | 100 | 23 | 74 |
| LNK047 | 2 | 5 | 9 | 20 | 65 | 88 | 1 | 2 |
| LNK048 | 0 | 0 | 0 | 0 | 0 | 0 | 0 | 0 |

| | % | % | % | % | % | % | % |
|---|---|---|---|---|---|---|---|
| | cleavage | cleavage | cleavage | cleavage | vcleavage | cleavage | cleavage |
| | MMP-9 | Matriptase | Matriptase | Matriptase | uPA | uPA | uPA |
| Linker | t = 120 min | t = 15 min | t = 60 min | t = 120 min | t = 15 min | t = 60 min | t = 120 min |
| LNK031 | 100 | 99 | 100 | 100 | 1 | 1 | 2 |
| LNK032 | 100 | 81 | 100 | 100 | 0 | 0 | 0 |
| LNK033 | 100 | 100 | 100 | 100 | 1 | 2 | 3 |
| LNK034 | 100 | 100 | 100 | 100 | 0 | 0 | 0 |
| LNK035 | 100 | 67 | 100 | 100 | 0 | 0 | 0 |
| LNK036 | 100 | 76 | 100 | 100 | 0 | 0 | 0 |
| LNK037 | 100 | 90 | 100 | 100 | 0 | 0 | 0 |
| LNK038 | 97 | 100 | 100 | 100 | 0 | 0 | 0 |
| LNK039 | 94 | 77 | 100 | 100 | 0 | 0 | 0 |
| LNK040 | 89 | 100 | 100 | 100 | 0 | 0 | 0 |
| LNK041 | 1 | 100 | 100 | 100 | 0 | 0 | 0 |
| LNK042 | 100 | 28 | 80 | 100 | 2 | 6 | 13 |
| LNK043 | 100 | 24 | 73 | 100 | 0 | 0 | 0 |
| LNK044 | 2 | 0 | 0 | 0 | 0 | 0 | 0 |
| LNK045 | 0 | 0 | 0 | 0 | 0 | 0 | 0 |
| LNK046 | 92 | 19 | 65 | 91 | 1 | 4 | 7 |
| LNK047 | 4 | 4 | 17 | 33 | 1 | 1 | 2 |
| LNK048 | 0 | 100 | 100 | 100 | 0 | 0 | 0 |

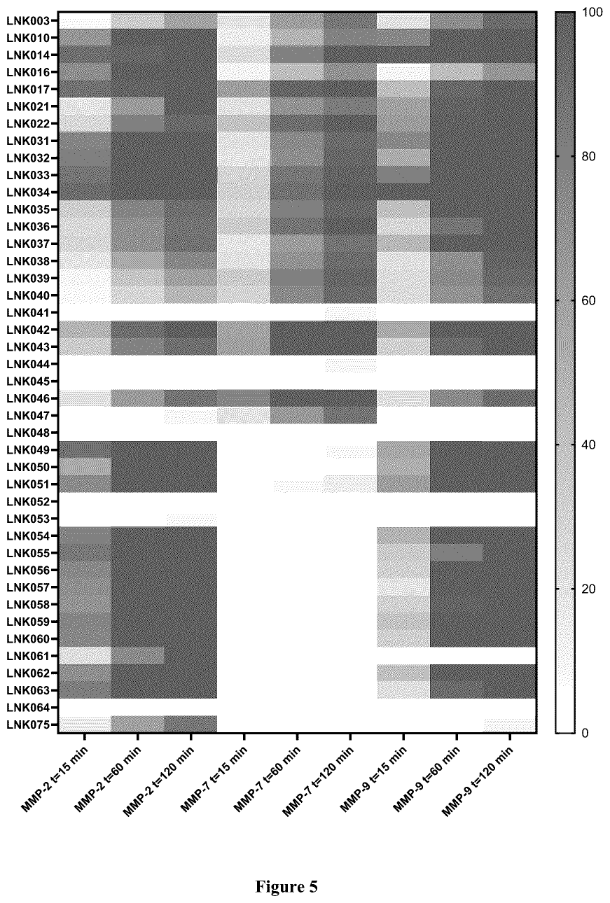

In addition to the above, selected peptides were further tested for cleavage efficiency with different MMPs. See Table 9 and FIG. 5.

TABLE 9
Cleavage Efficiency of selected peptides with MMPs.

| | % | % | % | % | % | % | % | % | % |
|---|---|---|---|---|---|---|---|---|---|
| | cleavage | cleavage | cleavage | cleavage | cleavage | cleavage | cleavage | cleavage | cleavage |
| | MMP-2 | MMP-2 | MMP-2 | MMP-7 | MMP-7 | MMP-7 | MMP-9 | MMP-9 | MMP-9 |
| | t = 15 | t = 60 | t = 120 | t = 15 | t = 60 | t = 120 | t = 15 | t = 60 | t = 120 |
| Linker | min | min | min | min | min | min | min | min | min |
| LNK003 | 7 | 34 | 59 | 19 | 62 | 86 | 22 | 72 | 93 |
| LNK010 | 67 | 100 | 100 | 15 | 49 | 78 | 76 | 99 | 100 |
| LNK014 | 91 | 96 | 100 | 25 | 79 | 100 | 98 | 99 | 100 |
| LNK016 | 72 | 100 | 100 | 11 | 43 | 70 | 10 | 43 | 67 |
| LNK017 | 89 | 97 | 100 | 62 | 96 | 100 | 43 | 94 | 100 |
| LNK021 | 20 | 64 | 100 | 22 | 69 | 83 | 60 | 98 | 100 |
| LNK022 | 27 | 79 | 94 | 40 | 0 | 100 | 63 | 99 | 100 |
| LNK031 | 77 | 100 | 100 | 22 | 74 | 93 | 75 | 100 | 100 |
| LNK032 | 81 | 100 | 100 | 20 | 72 | 95 | 53 | 100 | 100 |
| LNK033 | 87 | 100 | 100 | 30 | 86 | 98 | 81 | 100 | 100 |
| LNK034 | 92 | 100 | 100 | 32 | 89 | 100 | 100 | 100 | 100 |
| LNK035 | 33 | 77 | 91 | 28 | 80 | 95 | 42 | 100 | 100 |
| LNK036 | 27 | 70 | 88 | 34 | 88 | 100 | 28 | 87 | 100 |
| LNK037 | 28 | 70 | 89 | 19 | 63 | 88 | 46 | 100 | 100 |
| LNK038 | 19 | 55 | 76 | 22 | 69 | 92 | 23 | 71 | 97 |
| LNK039 | 10 | 38 | 61 | 35 | 80 | 96 | 25 | 74 | 94 |
| LNK040 | 8 | 28 | 47 | 29 | 74 | 93 | 25 | 69 | 89 |
| LNK041 | 0 | 0 | 0 | 1 | 4 | 9 | 0 | 1 | 1 |
| LNK042 | 49 | 91 | 100 | 59 | 100 | 100 | 55 | 100 | 100 |
| LNK043 | 34 | 78 | 91 | 61 | 100 | 100 | 32 | 93 | 100 |
| LNK044 | 0 | 0 | 1 | 1 | 4 | 9 | 0 | 1 | 2 |
| LNK045 | 0 | 0 | 0 | 0 | 0 | 0 | 0 | 0 | 0 |
| LNK046 | 19 | 64 | 88 | 77 | 100 | 100 | 23 | 74 | 92 |
| LNK047 | 2 | 5 | 9 | 20 | 65 | 88 | 1 | 2 | 4 |
| LNK048 | 0 | 0 | 0 | 0 | 0 | 0 | 0 | 0 | 0 |
| LNK049 | 89 | 100 | 100 | 1 | 4 | 8 | 56 | 100 | 100 |
| LNK050 | 54 | 100 | 100 | 1 | 3 | 6 | 52 | 100 | 100 |
| LNK051 | 73 | 100 | 100 | 3 | 8 | 14 | 62 | 100 | 100 |
| LNK052 | 1 | 2 | 4 | 0 | 0 | 0 | 0 | 0 | 0 |
| LNK053 | 2 | 5 | 8 | 0 | 0 | 0 | 1 | 3 | 4 |
| LNK054 | 78 | 100 | 100 | 0 | 0 | 0 | 49 | 100 | 100 |
| LNK055 | 85 | 100 | 100 | 0 | 0 | 0 | 35 | 81 | 100 |
| LNK056 | 75 | 100 | 100 | 0 | 0 | 0 | 32 | 100 | 100 |
| LNK057 | 71 | 100 | 100 | 0 | 0 | 0 | 20 | 100 | 100 |
| LNK058 | 68 | 100 | 100 | 0 | 0 | 0 | 31 | 97 | 100 |
| LNK059 | 77 | 100 | 100 | 0 | 0 | 0 | 39 | 100 | 100 |
| LNK060 | 76 | 100 | 100 | 0 | 0 | 0 | 32 | 100 | 100 |
| LNK061 | 23 | 74 | 100 | 0 | 0 | 0 | 0 | 0 | 0 |
| LNK062 | 71 | 100 | 100 | 0 | 0 | 0 | 41 | 100 | 100 |
| LNK063 | 82 | 100 | 100 | 2 | 3 | 0 | 23 | 93 | 100 |
| LNK064 | 0 | 0 | 0 | 0 | 0 | 0 | 0 | 0 | 0 |
| LNK075 | 16 | 60 | 86 | 0 | 0 | 0 | 1 | 5 | 10 |

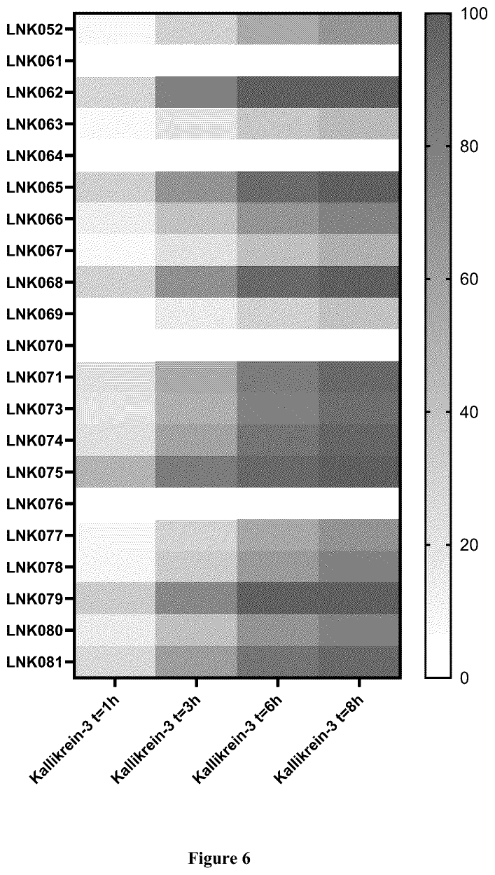

In addition to the above, certain peptide were further tested for cleavage efficiency with Kallikrein-3. See Table 10 and FIG. 6.

TABLE 10
Cleavage Efficiency of Select Peptides with Kallikrein-3

| | Kallikrein-3 | Kallikrein-3 | Kallikrein-3 | Kallikrein-3 |
|---|---|---|---|---|
| | t = 1 h | t = 3 h | t = 6 h | t = 8 h |
| | % | % | % | % |
| Linker | cleavage | cleavage | cleavage | cleavage |
| LNK052 | 8 | 31 | 55 | 67 |
| LNK061 | 0 | 0 | 0 | 0 |
| LNK062 | 28 | 79 | 100 | 100 |
| LNK063 | 7 | 20 | 37 | 45 |
| LNK064 | 0 | 0 | 0 | 0 |
| LNK065 | 32 | 68 | 93 | 100 |
| LNK066 | 14 | 40 | 67 | 78 |
| LNK067 | 8 | 22 | 41 | 51 |
| LNK068 | 31 | 70 | 94 | 100 |
| LNK069 | 6 | 16 | 31 | 40 |
| LNK070 | 0 | 0 | 0 | 0 |
| LNK071 | 23 | 55 | 83 | 94 |
| LNK073 | 20 | 51 | 79 | 91 |
| LNK074 | 23 | 59 | 87 | 96 |
| LNK075 | 49 | 83 | 94 | 100 |
| LNK076 | 0 | 0 | 0 | 0 |
| LNK077 | 9 | 30 | 56 | 69 |
| LNK078 | 10 | 34 | 64 | 79 |
| LNK079 | 34 | 75 | 99 | 100 |
| LNK080 | 16 | 41 | 68 | 79 |
| LNK081 | 27 | 62 | 87 | 94 |

### Example 3: Protease Cleavage of Protein (IL-2) with Cleavable Peptides and Activation Methods Rationale

A few selected cleavable peptide sequences (from Example 2) are further incorporated into an activatable IL-2 polypeptide (e.g., one of the constructs shown in Table 4, where the cleavable peptide sequence therein (e.g., the underlined portion) is replaced with one of the peptides provided in Example 1 (without the terminal FRET pairs). Such IL-2s are desirably selectively activated such that cleavage of the cleavable peptide results in enhanced binding of the IL-2 polypeptide to the IL-2 receptor (or a subunit thereof). The use of the best identified cleavable peptide sequences in Example 2 results in an IL-2 polypeptide which is better activated at or near a tumor microenvironment in vivo compared to other cleavable peptides which can be used, including those shown in Table 4. Enhanced cleavage is in some instances the result of multiple protease cleavage sites present on the cleavable peptide and/or enhanced activity of individual proteases on the designed sequences. Selected cleavable peptides are attached to the amino acid side chain of IL-2 via covalent bond during the synthesis. The experimental design is such that the IL-2 protein could be selectively activated after the cleavage of the cleavable peptide linkers. The experimental design was built on sequences from Table 2.

### Method

Cleavable peptide sequences are incorporated to the IL-2 polypeptide via chemical synthesis.

### Results

The IL-2 polypeptide is selectively activated in vitro by incubation with uPA (R&D System, 1310-SE), Matriptase (R&D Systems, 3946-SEB) and/or MMP2 (SIGMA, PF023) in a manner similar to that described in Example 3. The activity of the IL-2 polypeptides is then assessed by an activity assay. Based this data, top candidates are further assessed in vivo for anti-tumor activity, either as an activatable IL-2 alone or further conjugated to an anti-PD-1 antibody.

1-30. (canceled)

31. A cleavable peptide comprising a peptide of formula G³, wherein G³ has a structure:
*—X^{A1}—X^{A2}—X^{A3}—X^{A4}—X^{A5}—X^{A6}—X^{A7}—X^{A8}—X^{A9}—X^{A10}—**,
wherein:
X^{A1} is T, ornithine (Orn), H, S, R, or K;
X^{A2} is S, D, E, T, A, N, G, or Q;
X^{A3} is Orn, H, R, K, or absent;
X^{A4} is Orn, H, R, or K;
X^{A5} is L, G, I, Nle, M, V, or P;
X^{A6} is Y, F, V, I, Nle, M, Q, A, or L;
X^{A7} is S, D, E, T, M, P, Q, N, or G;
X^{A8} is Y, F, L, P, or absent;
X^{A9} is Y, F, T, L, I, Nle, M, Q, V, A, or S;
X^{A10} is Q, E, S, T, N, D, or G;
wherein * represents either the N-terminus of the cleavable peptide or a point of attachment to an additional group A¹; and ** represents the C-terminus of the cleavable peptide or a point of attachment to an additional group A¹; wherein each additional group A¹ can be the same additional group A¹ attached at multiple locations of the cleavable peptide or can be different additional groups A¹, and wherein the cleavable peptide comprises at least two protease cleavage sites.

32. The cleavable peptide of claim 31, wherein X^{A1} is T, Orn, H, S, R, or K; X^{A2} is A, N, G, or Q; X^{A3} is Orn, H, R, K, or absent; X^{A4} is Orn, H, R, or K; X^{A5} is L, G, I, Nle, M, V, or P; X^{A6} is V, I, Nle, M, Q, A, or L; X^{A7} is M, P, Q, N, or G; X^{A8} is L, P, or absent; X^{A9} is, T, L, I, Nle, M, Q, V, A, or S; and X^{A10} is N, D, or G.

33. The cleavable peptide of claim 31, wherein X^{A1} is S, R, or K; X^{A2} is G or Q; X^{A3} is R, K, or absent; X^{A4} is R or K; X^{A5} is V or P; X^{A6} is A or L; X^{A7} is N or G; X^{A8} is L or P; X^{A9} is V, A, or S; and X^{A10} is D or G.

34. The cleavable peptide of claim 31, wherein X^{A1} is S or R; X^{A2} is G; X^{A3} is R or absent; X^{A4} is R, X^{A5} is V; X^{A6} is A; X^{A7} is N; X^{A8} is L; X^{A9} is V; and X^{A10} is G.

35. The cleavable peptide of claim 31, wherein X^{A5} is V; X^{A6} is A; X^{A7} is N; X^{A8} is L; X^{A9} is V; and X^{A10} is G.

36. The cleavable peptide of claim 31, wherein X^{A1} is S, R, or K; X^{A2} is G or Q; X^{A3} is R, K, or absent; and X^{A4} is R or K.

37. The cleavable peptide of claim 31, wherein X^{A1} is S or R; X^{A2} is G; X^{A3} is R or absent; and X^{A4} is R.

38. The cleavable peptide of claim 37, wherein X^{A5} is V or P; X^{A6} is A or L; X^{A7} is N or G; X^{A8} is L or P; X^{A9} is V, A, or S; and X^{A10} is D or G.

39. The cleavable peptide of claim 31, wherein the cleavable peptide does not comprise the sequence PLGLAG (SEQ ID NO: 129).

40. The cleavable peptide of claim 31, wherein each of the additional groups A¹ is independently selected from a polypeptide, a nucleic acid, a polysaccharide, a lipid, an antibody, an organic biopolymer, a chemical polymer, a drug, a nanoparticle, a dye, or a bio-organic molecule.

41. The cleavable peptide of claim 31, comprising two different additional groups A¹.

42. The cleavable peptide of claim 41, wherein cleavage of any one of the at least two protease cleavage sites causes the two different additional groups A¹ to no longer be covalently linked.

43. The cleavable peptide of claim 31, comprising an additional group A¹ attached at two locations of the cleavable peptide to form a cyclic structure.

44. The cleavable peptide of claim 43, wherein cleavage of any one of the at least two protease cleavage sites breaks the cyclic structure.

45. The cleavable peptide of claim 31, wherein the cleavable peptide is cleavable by at least two proteases selected from a kallikrein, thrombin, chymase, carboxyprotease A, and elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease.

46. The cleavable peptide of claim 31, wherein each additional group A¹ is optionally connected to the cleavable peptide by a linker.

47-54. (canceled)

55. The cleavable peptide of claim 31, wherein the cleavable peptide comprises a sequence RQRR (SEQ ID NO: 405), RGRK (SEQ ID NO: 407), or RGRR (SEQ ID NO: 406).

56. (canceled)

57. (canceled)

58. The cleavable peptide of claim 1, wherein the cleavable peptide comprises: RGRRPLGLAG (SEQ ID NO: 349), RGRKVANLVG (SEQ ID NO: 331), RQRKVANLVG (SEQ ID NO: 332), RGRRVANLVG (SEQ ID NO: 333), or RGRKPQPLVD (SEQ ID NO: 339).

59-61. (canceled)

62. The cleavable peptide of claim 31, wherein the cleavable peptide is incorporated into the amino acid sequence of a protein.

63-66. (canceled)

67. The cleavable peptide of claim 31, wherein the cleavable peptide is attached to a side chain of an amino acid of a protein.

68. (canceled)
